(19) United States
(12) Patent Application Publication
(10) Pub. No.: US 2026/0132175 A1
(43) Pub. Date: May 14, 2026

(54) METHODS AND COMPOSITIONS TO MODULATE T CELL METABOLISM, FUNCTION, AND FATE

(71) Applicant: Cornell University, Ithaca, NY (US)

(72) Inventors: Juan R. Cubillos-Ruiz, New York, NY (US); Sung-Min Hwang, New York, NY (US)

(21) Appl. No.: 19/258,293

(22) Filed: Jul. 2, 2025

Related U.S. Application Data
(60) Provisional application No. 63/667,417, filed on Jul. 3, 2024.

(51) Int. Cl. C07K 14/47; A61K 40/11; A61K 40/31; A61K 40/42; A61P 35/04; C07K 14/705; C07K 14/725; C07K 16/26; C12N 5/0783

(52) U.S. Cl. CPC C07K 14/47; A61K 40/11; A61K 40/31; A61K 40/4237; A61P 35/04; C07K 14/7051; C07K 14/70517; C07K 14/70578; C07K 16/26; C12N 5/0638; A61K 2239/59; C07K 2317/24; C07K 2319/03; C12N 2510/00

(57) ABSTRACT

The present disclosure relates to compositions and methods for treating cancers (e.g., ovarian cancer) or increasing cytotoxicity of T cells by modulating expression, level, and/or activity of Transgelin-2 (TAGLN2) in T cells. It also discloses vectors to increase expression, level, and/or activity of TAGLN2 in T cells, T cells co-expressing a CAR and TAGLN2, and adaptive cell therapies using such T cells. Methods of assessing functional status and/or anti-tumor activity of T cells using TAGLN2 as a biomarker are also disclosed.

### RELATED APPLICATION

This application claims the benefit of U.S. Provisional Patent Application Ser. No. 63/667,417, filed on Jul. 3, 2024, which is hereby incorporated by reference herein in its entirety.

### GOVERNMENT SUPPORT

This invention was made with government support under W81XWH-16-1-0438 and W81XWH-23-1-0204 awarded by the Defense Health Agency, Medical Research and Development Branch, and CA282072 awarded by National Institutes of Health. The government has certain rights in the invention.

### SEQUENCE LISTING

The instant application contains a Sequence Listing which has been submitted electronically in XML format and is hereby incorporated by reference in its entirety. Said XML copy, created on Nov. 12, 2025, is named CUW-03601_SL.xml and is 49,350 bytes in size.

### BACKGROUND OF THE INVENTION

T cell function and fate are dictated by nutrient availability and the precise regulation of multiple metabolic pathways⁶⁻⁸. For instance, the efficient import, trafficking, and catabolism of extracellular fatty acids is crucial to fulfill the bioenergetic demands of effector and memory CD8⁺ T cells^{2,3,9,10}. The fatty acid binding protein 5 (FABP5) plays a pivotal role in this process as a dominant transporter of extracellular fatty acids that mitochondria utilize for energy production through fatty acid β-oxidation (FAO)^{11,12}. While the FABP5-FAO axis has been demonstrated to be critical for the development and maintenance of adaptive immunity to pathogens and tumors^{4,5,13}, the mechanisms regulating this major immunometabolic pathway remain largely unexplored.

Metastatic ovarian cancer (OvCa) is a prototypical immunosuppressive malignancy, known for its resistance to standard treatments and all forms of T cell-based immunotherapy¹⁴⁻¹⁶. This daunting clinical scenario indicates that unconventional, yet to be identified, mechanisms of T cell suppression remain actively engaged in the OvCa environment despite major treatment efforts. Indeed, T cells infiltrating ovarian tumors are normally retained in a dysfunctional state characterized by bioenergetic anomalies, aberrant activation of cellular stress responses, and negligible effector function that cannot be reversed through classical immunotherapeutic approaches¹⁷⁻¹⁹. Whether aggressive epithelial malignancies, such as OvCa, disrupt FABP5-mediated lipid metabolic programming in T cells to evade adaptive immune control is unknown. There remains a need for effective therapies, including adaptive immune therapies, for ovarian cancer such as metastatic ovarian cancer (OvCa).

### SUMMARY OF THE INVENTION

The present disclosure depends, at least in part, on the discovery that the cytoskeletal protein TAGLN2 cooperated with FABP5 to enable efficient fatty acid import and utilization by CD8⁺ T cells. It was found that TAGLN2 was silenced in dysfunctional intratumoral CD8⁺ T cells by intrinsic endoplasmic reticulum (ER) stress responses. It was demonstrated herein that preserving TAGLN2 expression enhances the lipid uptake, bioenergetic, and functional capacities of ER-stressed T cells and thus improved the effectiveness of adoptive cellular immunotherapy in mice with metastatic OvCa.

In one aspect, provided herein is a method of treating a subject afflicted with a cancer, comprising administering to the subject an agent that increases expression, level, and/or activity of Transgelin-2 (TAGLN2) in T cells.

In some embodiments, the T cells are CD8+ T cells. In some embodiments, the T cells are activated CD8+ T cells. In some embodiments, the T cells are effector CD8+ T cell and/or central memory CD8+ T cells. In some embodiments, the T cells are intratumoral T cells. In some embodiments, the T cells are ER-stressed T cells. In some embodiments, the cancer is metastatic ovarian cancer (OvCa). In some embodiments, the cancer is high-grade serous OvCa (HGSOC). In some embodiments, prior to the administration of the agent, the T cells have a reduced level of TAGLN2 compared to T cells from a healthy subject or a subject without the cancer. In some embodiments, prior to the administration of the agent, the T cells have a reduced surface level of FABP5 and/or reduced lipid uptake compared to T cells from a healthy subject or a subject without the cancer. In some embodiments, prior to the administration of the agent, the T have an increased expression of Xbp1s, Sec61a1a, and ERdj4 compared to T cells from a healthy subject or a subject without the cancer.

In one aspect, provided herein is a method of increasing cytotoxicity of a T cell comprising contacting the T cell with an agent that increases expression, level, and/or activity of Transgelin-2 (TAGLN2) in the T cell.

In some embodiments, the T cell expresses a chimeric antigen receptor (CAR). In some embodiments, the CAR is a chimeric endocrine receptor (CER). In some embodiments, the chimeric endocrine receptor (CER) comprises: a) the β subunit of the follicle-stimulating hormone (FSHβ), b) a spacer, c) a transmembrane domain, and d) an intracellular domain. In some embodiments, the FSHβ comprises a FSHβ signal peptide at the 5′ end. In some embodiments, the spacer is a glycine/serine spacer. In some embodiments, the transmembrane domain is a CD8α transmembrane domain. In some embodiments, the intracellular domain is a 4-1BB-CD3ζ (BBZ) intracellular domain (ICD). In some embodiments, the T cell is a cytotoxic T cell. In some embodiments, the T cell is a primary CD8+ T cell. In some embodiments, the T cells are ER-stressed T cells. In some embodiments, the method increases the cytotoxicity of the T cell in vitro or in vivo. In some embodiments, the method increases the cytotoxicity of the T cell in a subject afflicted with cancer. In some embodiments, the cancer is metastatic ovarian cancer (OvCa). In some embodiments, the cancer is high-grade serous OvCa (HGSOC). In some embodiments, the agent is a small molecule compound, a RNA, a DNA, or a protein. In some embodiments, the agent reduces expression, level, and/or activity of IRE1α. In some embodiments, the agent is a IRE1α inhibitor. In some embodiments, the IRE1α inhibitor is MKC8866 or KIRA8. In some embodiments, the agent reduces expression, level, and/or activity of XBP1s. In some embodiments, the agent increases expression, level, and/or activity of NF-kB in T cells. In some embodiments, the agent is a Tagln2 mRNA or a TAGLN2 protein. In some embodiments, the agent increases cell surface level of FABP5 in the T cells. In some embodiments, the agent increases fatty acid uptake of the T cells. In some embodiments, the agent enhances mitochondrial respiration. In some embodiments, the agent enhances microtubule dynamics, organization of cytoskeleton, proliferation, and/or activation of the T cells. In some embodiments, the agent increases expression of Ki-47 and/or CD44.

In some aspects, provided herein is a vector comprising (1) a first nucleic acid encoding a chimeric antigen receptor (CAR); and (2) a second nucleic acid encoding TAGLN2. In some embodiments, the CAR is a chimeric endocrine receptor (CER). In some embodiments, the chimeric endocrine receptor (CER) comprises: a) the 3 subunit of the follicle-stimulating hormone (FSHβ), b) a spacer, c) a transmembrane domain, and d) an intracellular domain. In some embodiments, the FSHβ comprises a FSHβ signal peptide at the 5′ end. In some embodiments, the spacer is a glycine/serine spacer. In some embodiments, the transmembrane domain is a CD8α transmembrane domain. In some embodiments, the intracellular domain is a 4-1BB-CD3ζ (BBZ) intracellular domain (ICD). In some embodiments, the second nucleic acid is a cDNA encoding TAGLN2. In some embodiments, the second nucleic acid is at the 3′ downstream of the first nucleic acid. In some embodiments, the vector further comprises a IRES between the first nucleic acid and the second nucleic acid. In some embodiments, the vector further comprises a reporter gene. In some embodiments, the vector is an expression vector. In some embodiments, the vector is a viral vector. In some embodiments, the viral vector is lentiviral vector or a retroviral vector. In some embodiments, the vector is the CER-Tagln2 RV in FIG. 6E. In some aspects, provided herein is a T cell comprising the vector described herein. In some embodiments, the T cell expresses the CAR and TAGLN2. In some aspects, provided herein is a T cell expressing a chimeric antigen receptor (CAR) and comprising a transgene encoding TAGLN2. In some embodiments, the CAR is a chimeric endocrine receptor (CER). In some embodiments, the chimeric endocrine receptor (CER) comprises: a) the 3 subunit of the follicle-stimulating hormone (FSHβ), b) a spacer, c) a transmembrane domain, and d) an intracellular domain. In some embodiments, the FSHβ comprises a FSHβ signal peptide at the 5′ end. In some embodiments, the spacer is a glycine/serine spacer. In some embodiments, the transmembrane domain is a CD8α transmembrane domain. In some embodiments, the intracellular domain is a 4-1BB-CD3ζ (BBZ) intracellular domain (ICD). In some embodiments, the T cell expresses TAGLN2. In some embodiments, the T cell has higher surface level of FABP5, a higher lipid uptake, and/or a higher cytotoxicity compared to a T cell without the transgene encoding TAGLN2. In some embodiments, the T cell is a cytotoxic T lymphocyte (CTL). In some embodiments, the T cell is a primary CD8+ T cell.

In some aspects, provided herein is a composition comprising T cells described herein.

In some aspects, provided herein is a cell bank comprising T cells for adoptive immunotherapy, wherein the T cells are T cells described herein.

In some aspects, provided herein is a method of treating a cancer in a subject, the method comprising administering a composition comprising T cells described herein.

In some embodiments, the cancer is metastatic ovarian cancer. In some embodiments, the cancer is high-grade serous OvCa (HGSOC). In some embodiments, the subject has reduced peritoneal carcinomatosis after the treatment. In some embodiments, the subject has enhanced T cell infiltration into metastatic omental lesions after the treatment. In some embodiments, the subject has a prolonged survival after the treatment.

In some aspects, provided herein is a method of generating a CAR-expressing T cell comprising contacting the immune cell with (1) a first nucleic acid encoding a CAR; and (2) a second nucleic acid encoding TAGLN2. In some embodiments, the CAR is a chimeric endocrine receptor (CER).

In some embodiments, the chimeric endocrine receptor (CER) comprises: a) the β subunit of the follicle-stimulating hormone (FSHβ), b) a spacer, c) a transmembrane domain, and d) an intracellular domain. In some embodiments, the FSHβ comprises a FSHβ signal peptide at the 5′ end. In some embodiments, the spacer is a glycine/serine spacer. In some embodiments, the transmembrane domain is a CD8α transmembrane domain. In some embodiments, the intracellular domain is a 4-1BB-CD3ζ (BBZ) intracellular domain (ICD). In some embodiments, the second nucleic acid is a cDNA encoding TAGLN2. In some embodiments, the first nucleic acid and the second nucleic acid are in the same vector. In some embodiments, the method comprises contacting the immune cell with a vector described herein. In some embodiments, the first nucleic acid and the second nucleic acid are in two separate vectors. In some embodiments, the T cell expresses the CAR and TAGLN2. In some embodiments, the T cell is a cytotoxic T lymphocyte (CTL). In some embodiments, the T cell is a primary CD8+ T cell.

In some aspects, provided herein is a method of increasing lipid uptake of a T cell comprising contacting the T cell with an agent that increases expression, level, and/or activity of Transgelin-2 (TAGLN2) in T cells.

In some aspects, provided herein is a method of modulating T cell metabolism comprising contacting the T cell with an agent that increases expression, level, and/or activity of Transgelin-2 (TAGLN2) in T cells.

In some embodiments, the T cell metabolism is a T cell lipid metabolism.

In some aspects, provided herein is a method of assessing functional status and/or anti-tumor activity of T cells comprising detecting expression and/or level of TAGLN2 in the T cells and comparing it to a control, wherein the T cells are functional and/or have anti-tumor activity if they have the same or higher expression and/or level of TAGLN2 compared to the control, and the T cells are less functional and/or have reduced anti-tumor activity if they have less expression and/or level of TAGLN2 compared to the control.

In some embodiments, the T cells are endogenous intratumoral T cells. In some embodiments, the T cells are adaptively transferred intratumoral T cells. In some embodiments, the functional status of T cells comprises lipid uptake, lipid metabolism, mitochondrial respiration, microtubule dynamics, organization of cytoskeleton, proliferation, and/or activation of the T cells. In some embodiments, the anti-tumor activity of T cells comprises cytotoxicity of T cells. In some embodiments, the method is carried out in vivo or ex vivo. In some embodiments, the control is a healthy subject or a subject without a cancer. In some embodiments, the control is a reference number. In some embodiments, the method further comprises administering to a subject an agent that increases expression, level, and/or activity of Transgelin-2 (TAGLN2) in T cells if the T cells are less functional and/or have reduced anti-tumor activity.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1A-FIG. 1K show the defective lipid uptake and FABP5 surface localization in OvCa-infiltrating CD8⁺ T cells. FIG. 1A, Representative FACS histograms and quantitative analysis of lipid uptake (BODIPY 500/510) for CD45⁺CD19⁻CD3⁺CD8⁺ T cells isolated from peripheral blood of cancer-free women (n=14), malignant ascites (n=15) or peripheral blood (n=8) of HGSOC patients. FIG. 1B, FIG. 1C, Human naïve CD8⁺ T cells from peripheral blood of cancer-free women were activated via CD3/CD28 stimulation for 32 h, followed by 16 h in the absence or presence of 50% HGSOC ascites supernatants. (FIG. 1B) Lipid uptake was assessed by FACS (n=5). (FIG. 1C) FABP5, CD36 and FABP4 expression was determined via qRT-PCR and data were normalized to endogenous levels of ACTB (n=5). FIG. 1D-FIG. 1F, Human naïve CD8⁺ T cells from peripheral blood of cancer-free individuals were activated via CD3/CD28 stimulation and then Neon-electroporated with either control (Ctrl) or human FABP5 mRNAs. Cells were expanded and treated with 0% or 50% of HGSOC ascites supernatants. Representative histograms and quantitative analysis of total FABP5 protein levels (FIG. 1D), lipid uptake (FIG. 1E), and cell surface FABP5 protein levels (FIG. 1F) in CD8⁺CD44⁺ T cells determined by FACS (n=5 per group). FIG. 1G, FIG. 1H, Representative FACS histograms and quantitative analysis of cell surface (FIG. 1G) and total (FIG. 1H) FABP5 protein levels in CD45⁺CD19⁻CD3⁺CD8+ T cells isolated from the same specimens described in FIG. 1A. FACS-based analysis to determine the kinetics of cell surface FABP5 protein levels (FIG. 1I), total FABP5 protein levels (FIG. 1J), and lipid uptake (FIG. 1K) in CD45⁺CD19⁻CD3⁺CD8⁺ T cells from peritoneal lavage or omentum on days 7, 14, or 28 after tumor implantation (n=3 per group). Data are presented as mean±s.e.m. FIG. 1A, FIG. 1D-FIG. 1H, One-way ANOVA with Tukey's multiple comparisons test. FIG. 1B, Two-tailed paired Student's t-test. FIG. 1C, Two-tailed unpaired Student's t-test. Exact P-values are shown. The ‘n’ values represent biologically independent samples. gMFI, Geometric mean fluorescence intensity.

FIG. 2A-FIG. 2N show that TAGLN2 is required for FABP5 surface localization and lipid uptake in activated CD8⁺ T cells. FIG. 2A, Protein-protein interaction maps generated by the STRING database using FABP5 as query in human (Homo sapiens) and mouse (Mus musculus). FIG. 2B, FIG. 2C, Schematic of the immunoprecipitation coupled to mass spectrometry (IP-MS) approach to identify FABP5-interacting proteins. A total of 2,455 proteins were identified at a 1% FDR. Relative intensity was used to compare protein abundance across samples. The top 10 proteins that significantly interact with FABP5 are listed (FIG. 2B). The abundance of predicted FABP5-interacting proteins from (FIG. 2A) is listed by relative intensity obtained from IP-MS analysis in FIG. 2B (FIG. 2C). N.D, not detected. FIG. 2D, Interaction between FABP5 and TAGLN2 assessed by co-immunoprecipitation using activated CD8⁺ T cells from C57BL/6J mice. Representative image from three independent experiments is shown. FIG. 2E, FIG. 2F, Confocal images of activated CD8⁺ T cells from Tagln2^{fl/fl} or Tagln2^{fl/fl}Cd4^{Cre} mice stained for TAGLN2 (green) and FABP5 (red). Nuclei are depicted in blue (DAPI staining) (FIG. 2E). The percentage of colocalization of indicated markers is shown in (FIG. 2F) (Manders' coefficient). FIG. 2G, Wild-type naïve CD8⁺ T cells from the spleen and lymph nodes were activated via CD3/CD28 stimulation for 24 h and then Neon-electroporated with either control (Ctrl) or mouse Tagln2 mRNAs. After 48 h, representative histograms and quantitative analysis of surface FABP5 in CD8⁺CD44⁺ T cells determined by FACS (n=3 per condition). FIG. 2H, FIG. 2I, Wild-type (WT) or FABP5-deficient naïve CD8⁺ T cells from the spleen and lymph nodes were activated via CD3/CD28 stimulation for 24 h and then Neon-electroporated with either control (Ctrl) or mouse Tagln2 mRNAs. Experimental scheme and readouts (FIG. 2H). Representative histograms and quantitative analysis of lipid uptake (BODIPY 500/510) in CD8⁺CD44⁺ T cells determined by FACS (n=3 per condition) (FIG. 2I). FIG. 2J, FIG. 2K, Wild-type (WT), TAGLN2-deficient (Tagln2^{KO}) or FABP5-deficient (Fabp5^{KO}) naïve CD8⁺ T cells from the spleen and lymph nodes were activated via CD3/CD28 stimulation for 24 h and then Neon-electroporated with either control (Ctrl) or mouse Tagln2 mRNAs. After 48 h, lipidomic analyses were performed to evaluate the levels of diverse fatty acid species in indicated CD8⁺ T cells. Relative abundance (% of total fatty acid content) of total fatty acids (FIG. 2J) and quantification of palmitoleic acid (left) and oleic acid (right) (FIG. 2K) are shown (n=3 per condition). FIG. 2L, Relative fatty acid abundance in malignant ascites from mice bearing advanced ID8-Defb29/Vegfa ovarian cancer. Major fatty acids in this ascites are indicated in the box. FIG. 2M, FIG. 2N, Highlight the relative abundance (FIG. 2M) and quantification (FIG. 2N) of palmitoleic acid and oleic acid in the indicated T cell conditions (n=3 per condition). The same ascites used in 1. Data are presented as mean±s.e.m. FIG. 2F, FIG. 2G, Two-tailed unpaired Student's t-test. FIG. 2I, FIG. 2K, FIG. 2N, One-way ANOVA with Tukey's multiple comparisons test. Exact P-values are shown. The ‘n’ values represent biologically independent samples. gMFI, Geometric mean fluorescence intensity.

FIG. 3A-FIG. 3K show the status of TAGLN2 in OvCa-infiltrating CD8⁺ T cells. FIG. 3A, Representative FACS histograms and quantitative analysis of TAGLN2 protein levels in CD45⁺CD19⁻CD3⁺CD8⁺ T cells isolated from peripheral blood of cancer-free women (n=14), malignant ascites (n=15) or peripheral blood (n=8) of HGSOC patients. FIG. 3B, Representative FACS plot of CD8⁺ T cell-subset analysis from malignant ascites of HGSOC patients (left). TAGLN2 protein expression was quantified in naïve (pink), effector (brown), effector memory (green) and central memory (orange) CD8⁺ T cells, respectively (right) (n=15). Cells were pre-gated on CD45⁺CD19⁻CD3⁺CD8⁺ T cells. naïve, CCR7⁺CD45RO⁻; effector, CCR7-CD45RO⁻; effector memory, CCR7-CD45RO⁺; central memory, CCR7⁺CD45RO⁺. FIG. 3C, Correlation analysis for IFNG, TNFA or GZMB versus TAGLN2 mRNA in CD8⁺ T cells from malignant ascites of HGSOC patients of. Data were normalized to ACTB in all cases (n=15). FIG. 3D, Correlation of IFN-γ concentration versus levels of TAGLN2 in the indicated CD8⁺ T cell subsets in ascites of HGSOC patients (n=14). FIG. 3E, Naïve CD8⁺ T cells from peripheral blood of cancer-free women were activated via CD3/CD28 stimulation for 32 h and then incubated for 16 h with increasing amounts of HGSOC ascites supernatants (n=5). Expression of TAGLN2, IFNG, and GZMB was assessed by qRT-PCR. Data were normalized to ACTB. FIG. 3F, Representative FACS histograms and quantitative analysis of TAGLN2 protein levels in effector (CD62L^{low}CD44high) or central memory (CD62L^{high}CD44high) CD8⁺ T cells from peritoneal wash of cancer-free mice (n=8) or malignant ascites of female mice bearing ID8-Defb29/Vegfa OvCa (n=6). FIG. 3G, FIG. 3H, Representative FACS histograms and quantitative analysis of CD44 (FIG. 3G) and Ki-67 (FIG. 3H) expression in TAGLN2^{low} or TAGLN2high CD45⁺CD19⁻CD3⁺CD8⁺ T cells from malignant ascites of female mice bearing ID8-Defb29/Vegfa OvCa (n=6 per group). FIG. 3I-FIG. 3K, Representative FACS plots and quantitative analysis of CD44+IFN-γ⁺ (i), CD44+ TNF-+(FIG. 3J) and CD44⁺GZMB⁺ (FIG. 3K) frequencies in TAGLN2^{low} or TAGLN2high CD45⁺CD19⁻CD3⁺CD8⁺ T cells from the same mice described in (FIG. 3G) and (FIG. 3H). Data are presented as mean±s.e.m. FIG. 3A, FIG. 3F-FIG. 3H, Two-tailed unpaired Student's t-test. FIG. 3B, FIG. 3E, One-way ANOVA with Tukey's multiple comparisons test. FIG. 3C, FIG. 3D, Spearman's rank correlation test, Spearman coefficient (r) with exact P-value (two-tailed). FIG. 3I-FIG. 3K, Two-tailed paired Student's t-test. Exact P-values are shown. The ‘n’ values represent biologically independent samples. gMFI, Geometric mean fluorescence intensity.

FIG. 4A-FIG. 4Q show that XBP1s restrains TAGLN2 expression in ER-stressed CD8+ T cells. FIG. 4A, Pre-activated CD8⁺ T cells from WT C57BL/6J mice were treated with 2-Deoxy-D-glucose (2-DG), Tunicamycin (TM) or Thapsigargin (TG) at the indicated concentrations. Xbp1s and Tagln2 expression were determined via qRT-PCR 16 h after 2-DG or TM treatment, and 6 h post TG exposure. Data were normalized to Actb in each sample (n=3 per condition). FIG. 4B, TAGLN2 protein expression levels determined by FACS (n=3 per group) in the same samples described in (FIG. 4A). FIG. 4C, Representative confocal images for TAGLN2 expression in CD8⁺ T cells from WT C57BL/6J mice under the indicated conditions from two independent experiments. FIG. 4D-FIG. 4G, Naïve CD8⁺ T cells isolated from Ern1^{fl/fl} or Em1^{fl/fl} Cd4^{Cre} mice (FIG. 4D, FIG. 4E) or Xbp1^{fl/fl} or Xbp1^{fl/fl}Cd4^{Cre} mice (FIG. 4F, FIG. 4G) were cultured under the indicated conditions. Expression of the Tagln2 transcript was determined by RT-qPCR, and data were normalized to endogenous levels of Actb in each sample (FIG. 4D, FIG. 4F) (n=3 per condition and genotype). Representative FACS histograms and quantitative analysis of TAGLN2 protein levels in CD8⁺CD44⁺ T cells under the indicated conditions (FIG. 4E, FIG. 4G) (n=3 per genotype). FIG. 4H, Pre-activated CD8⁺ T cells from WT C57BL/6J mice were stimulated via CD3/CD28 for 16 h in the absence or presence of TM (1 μg/ml). White bars, DMSO; Red bars, MKC8866 (2 μM); Orange bars, KIRA8 (1 μM). Expression of Xbp1s and Tagln2 transcripts were determined by RT-qPCR, and data were normalized to endogenous levels of Actb in each sample (n=3 per condition). FIG. 4I, Conserved XBP1s-binding motifs (CACGTC) from mouse (top) and human (bottom) are shown. FIG. 4J, Pre-activated CD8⁺ T cells from WT C57BL/6J mice were stimulated via CD3/CD28 for 16 h in the absence or presence of the ER stressor TM (1 μg/ml). ChIP assays were performed using anti-XBP1s or isotype control antibodies. qRT-PCR was used to determine XBP1s occupancy at two XBP1s-binding sites (BS1 and BS2) in Tagln2 promoter regions under the conditions tested. No XBP1s binding region (NR) was used as a negative control. ChIP-quantitative PCR assays were performed using T cells from three independent mice (n=3 per condition). FIG. 4K, Tagln2 promoter-luciferase construct (−738 to +134) was co-transfected with the combination of NF-kB (p50) and XBP1s expressing vectors in HEK-293 T cells. Lysates were prepared 48 h after transfection, and luciferase activities were measured with the firefly luciferase activities normalized to renilla luciferase activities (n=3). FIG. 4L, FIG. 4M, Representative FACS histograms and quantitative analysis of TAGLN2 protein levels in effector (CD62L^{low}CD44high) or central memory (CD62L^{high}CD44high) CD8+ intratumoral T cells in omentum (FIG. 4L) and solid tumor (FIG. 4M) from female mice of the indicated genotypes bearing PPNM-based HGSC for 40 days (Xbp1^{fl/fl}, n=5; Xbp1^{fl/fl}Cd4^{Cre}, n=8). FIG. 4N, UMAP plot of T cell subtypes from 11 HGSOC treatment-naïve human tumor specimens. FIG. 4O, GSEA enrichment plots showing downregulation of ER stress gene signature in TAGLN2^{hi} CD8+ tumor-infiltrating effector memory T cells (T_{EM}). NES, normalized enrichment score. FDR, false discovery rate. FIG. 4P, Correlation analysis for Xbp1s versus TAGLN2 mRNA expression levels in CD8⁺ T cells residing in the ascites of HGSOC patients. Data were normalized to ACTB (n=16). FIG. 4Q, Correlation of XBP1s versus TAGLN2 protein expression in CD45⁺CD19⁻CD3⁺CD8⁺ T cells from the ascites of HGSOC patients (n=14). Data are presented as mean±s.e.m. FIG. 4A, FIG. 4B, FIG. 4D, FIG. 4F, FIG. 4H, FIG. 4K, One-way ANOVA with Tukey's multiple comparisons test. FIG. 4E, FIG. 4G, FIG. 4J, FIG. 4L, FIG. 4M, Two-tailed unpaired Student's t-test. FIG. 4P, FIG. 4Q, Spearman's rank correlation test, Spearman coefficient (r) with exact P-value (two-tailed). P<0.05 is considered statistically significant and exact P-values are shown. The ‘n’ values represent biologically independent samples. gMFI, Geometric mean fluorescence intensity.

FIG. 5A-FIG. 5M show that TAGLN2 overexpression rescues the bioenergetic defects of ER-stressed CD8⁺ T cells. FIG. 5A, Pre-activated CD8⁺ T cells from WT C57BL/6J mice were stimulated via CD3/CD28 for 16 h in the absence or presence of TM at the indicated concentrations. Representative FACS histograms and quantitative analysis of cell surface FABP5 expression. FIG. 5B, Pre-activated CD8⁺ T cells from WT mice were treated with Tunicamycin™, 2-Deoxy-D-glucose (2-DG) or Thapsigargin (TG) at the indicated concentrations. Representative FACS histograms and quantitative analysis of lipid uptake in CD8⁺CD44⁺ T cells (n=3 per condition). FIG. 5C, FIG. 5D, Pre-activated CD8⁺ T cells from mice of the indicated genotypes were stimulated via CD3/CD28 for 16 h in the absence or presence of TM (1 μg/ml). Representative FACS histograms and quantitative analysis of cell surface FABP5 expression (FIG. 5C) and lipid uptake (FIG. 5D) in CD8⁺CD44⁺ T cells. FIG. 5E, Representative OCR plots (left) and quantification (right) of basal (blue) and maximal respiration (red) are shown. Data were normalized to total genomic DNA content in each condition (n=5). FIG. 5F-FIG. 5J, Naïve CD8⁺ T cells from WT C57BL/6J mice were stimulated via CD3/CD28 for 24 h and then Neon-electroporated with either Control (Ctrl) or mouse Tagln2 mRNAs. Electroporated T cells were maintained under CD3/CD28 stimulation for an additional 32 h and then treated with the ER stressor TM (1 μg/ml) for 16 h. Representative FACS histograms and quantitative analysis of lipid uptake in CD8⁺CD44⁺ T cells determined by FACS (n=3 per condition) (FIG. 5F). Representative OCR plots (left) are shown (FIG. 5G). Rates of basal respiration (middle) and maximal respiratory capacity (right) were quantified and normalized to total genomic DNA content (n=5 per condition). Expression levels of CD44 (left), Ki-67 (right) (n=9 per condition) (FIG. 5H) and quantitative analysis of CD44+IFN-γ⁺ (FIG. 5I) and CD44⁺GZMB⁺(FIG. 5J) frequencies in CD8⁺ T cells electroporated with the indicated mRNAs and exposed to TM (n=5 per condition). FIG. 5K, OCR of TM-treated Tagln2-overexpressing CD8⁺ T cells in response to media (vehicle) or etomoxir injection. The maximal respiratory capacity was quantified and normalized to total genomic DNA content (n=5 per condition). FIG. 5L, Expression levels of CD44 (left) and Ki-67 (right) in TM-treated Tagln2-overexpressing CD8⁺ T cells in the absence or presence of exogenous oleic acid (OA, 30 μM). FIG. 5M, WT or TAGLN2-deficient naïve CD8⁺ T cells from spleen and lymph nodes were activated via CD3/CD28 stimulation in complete medium for 24 h, followed by 48 h culture with or without oleic acid in either complete- or glucose-free medium. Representative FACS plots and quantitative analysis of mitochondrial membrane potential analyzed by MitoTracker Deep Red staining (n=4 per condition). Data are presented as mean±s.e.m. FIG. 5A, FIG. 5F-FIG. 5L, Two-tailed paired Student's t-test. FIG. 5C, FIG. 5D, Two-tailed unpaired Student's t-test. FIG. 5B, FIG. 5M, One-way ANOVA with Tukey's multiple comparisons test. P<0.05 is considered statistically significant and exact P-values are shown. The ‘n’ values represent biologically independent samples. gMFI, Geometric mean fluorescence intensity.

FIG. 6A-FIG. 6N show that preserving TAGLN2 enhances the therapeutic effects of CER T cells in OvCa. FIG. 6A, CER T cells were electroporated with the indicated mRNAs and then treated with vehicle or TM (1 μg/ml) for 16 h. T cells were washed to remove TM and then cocultured with PPNM cancer cells at a 1:1 ratio. Cancer cell death was assessed via Annexin V and PI staining by FACS 18 h later. Representative FACS plots and quantitative analysis (n=3-4 per condition). FIG. 6B, Schematics of retroviral CER expression constructs. FSHP, Follicle-stimulating hormone beta subunit. CG(a, chorionic gonadotropin alpha subunit. IRES, Internal ribosome entry site. GFP, Green fluorescent protein. FIG. 6C, Experimental scheme for adoptive transfer of CER or CER-Tagln2 T cells into PPNM tumor-bearing WT C57BL/6J female mice. FIG. 6D-FIG. 6I, Expression of TAGLN2 protein (FIG. 6D), frequencies of CD62L^{hi}CD44^{hi} (central memory) (FIG. 6e), cell surface and total levels of FABP5 protein (FIG. 6F), and CD44+IFN-γ⁺ (FIG. 6G), CD44+ TNF-α+(FIG. 6H) and CD44⁺GZMB⁺(i) were assessed in the indicated CER T cell populations from peritoneal lavage or omentum at day 21 of tumor development (7 days after the second T cell infusion). Representative FACS histograms and quantitative analysis are shown (n=3-8 mice per group). FIG. 6J, FIG. 6K, Peritoneal carcinomatosis in female mice bearing PPNM-based HGSC and treated with the indicated CER T cells. Representative bioluminescence images of PPNM tumors over time (FIG. 6J) and quantification of peritoneal tumor burden (FIG. 6K) in the indicated groups (n=16 mice per group). FIG. 6L, FIG. 6M, Representative images of omentum (FIG. 6L), and quantification of CD3⁺ T cell infiltration into omental samples (FIG. 6M) in the indicated female mice bearing PPNM tumors (n=8 per group). FIG. 6N, Overall survival rates for the mice described in (FIG. 6J, FIG. 6K) (n=16 per group). Data are presented as mean±s.e.m. FIG. 6A, FIG. 6K, One-way ANOVA with Tukey's multiple comparisons test. FIG. 6D-FIG. 6I, Two-tailed unpaired Student's t-test. FIG. 6M, Fisher's exact test was used to determine the association between the two categorical variables. FIG. 6N, Log-rank test for survival. P<0.05 is considered statistically significant and exact P-values are shown. The ‘n’ values represent biologically independent samples. gMFI, Geometric mean fluorescence intensity.

FIG. 7A-FIG. 7M show that FABP5 surface localization defect in OvCa-TME is specific to CD8⁺ T cells. FIG. 7A, Representative FACS histograms and quantitative analysis of lipid uptake (BODIPY 500/510) for CD45⁺CD19⁻CD3⁺CD8⁺ T cells isolated from peripheral blood of cancer-free women (n=6), matched peripheral blood (n=4) and malignant solid tumors (n=4) of HGSOC patients. FIG. 7B, FIG. 7C, Human naïve CD8⁺ T cells from peripheral blood of cancer-free women were activated via CD3/CD28 stimulation for 32 h, followed by 16 h in the absence or presence of 50% PBS. (FIG. 7B) Lipid uptake was assessed by FACS (n=5). (FIG. 7C) FABP5, CD36 and FABP4 expression was determined via qRT-PCR and data were normalized to endogenous levels of ACTB (n=5). FIG. 7D, Scheme illustrating the different strategies to detect cell surface or total (surface+intracellular) FABP5 protein expression in activated CD8⁺ T cells from both human and mouse. Representative FACS histograms depict FABP5 staining, with gray peaks representing isotype controls. FIG. 7E, FIG. 7F, Representative histograms and quantitative analysis of cell surface (FIG. 7E) and total (FIG. 7F) levels of FABP5 protein expression determined by FACS in gated CD8⁺CD44⁺ T cells from WT or Fabp5knockout (KO) mice (n=4 per genotype). FIG. 7G-FIG. 7J, PBMCs from cancer-free women were cultured in the absence or presence of 50% HGSOC ascites supernatants for 16 h. Representative FACS histograms and quantitative analysis of cell surface and total FABP5 protein levels in CD45⁺CD19⁻CD3⁺CD8⁺ T cells (FIG. 7G), CD45⁺CD19+ B cells (FIG. 7H), CD45⁺CD11c⁺ myeloid cells (i) and CD45⁺CD19⁻CD11c⁻CD3+γ/δTCR⁺ T cells (FIG. 7J). FIG. 7K-FIG. 7M, C57BL/6J female mice (n=9) were intraperitoneally injected with ID8-Defb29/Vegfa OvCa cells and euthanized on days 7, 14, or 28 after tumor implantation (n=3 per group). Experimental scheme and readouts (FIG. 7K). Representative images of peritoneal lavage (FIG. 7L) and omentum (FIG. 7M) from each group. Data are presented as mean±s.e.m. FIG. 7A, One-way ANOVA with Tukey's multiple comparisons test. FIG. 7B, FIG. 7G-FIG. 7J, Two-tailed paired Student's t-test. c, Two-tailed unpaired Student's t-test. FIG. 7E, FIG. 7F, Two-tailed unpaired Student's t-test. P<0.05 is considered to be statistically significant and exact P-values are shown. The ‘n’ values represent biologically independent samples. gMFI, Geometric mean fluorescence intensity.

FIG. 8A-FIG. 8J shows characterization of mice lacking TAGLN2 in T cells and analysis of lipid uptake in Fabp5-silenced CD8 T cells overexpressing Tagln2. FIG. 8A, Description of the Talgln2 deletion strategy depicting floxed and deleted alleles. FIG. 8B, FIG. 8C, Deletion efficiency was analyzed in activated CD4+ or CD8⁺ T cells from Tagln2^{fl/fl} or Tagln2^{fl/fl}Cd4^{Cre} mice via qRT-PCR using a primer set that specifically detects the exon 3 region of Tagln2. Data were normalized to Actb (FIG. 8B). The intracellular levels of TAGLN2 protein were evaluated by FACS (c) (n=3 per genotype). FIG. 8D, Representative histograms and quantitative analysis of TAGLN2 protein expression in B cells (CD3-CD19+), natural killer (NK) cells (CD3-NK1.1+), dendritic cells (CD3-CD11c⁺MHC11+) and macrophages (CD3-CD11b+F4/80+) in the spleen and peripheral lymph nodes of Tagln2^{fl/fl} or Tagln2^{fl/fl} Cd4^{low} mice (n=3 per genotype). FIG. 8E, Representative FACS plots and quantitative analysis of double negative (CD4-CD8-), double positive (CD4⁺CD8+), or single positive (CD4+ or CD8+) thymocytes frequencies and absolute number in the thymus (n=5 per genotype). FIG. 8F, Representative FACS plots and quantitative analysis of CD3⁺CD4+ or CD3⁺CD8⁺ T cell frequencies and absolute number in the spleen (left) and peripheral lymph nodes (right) (n=5 per genotype). FIG. 8G, FIG. 8H, Expression of CD44 and CD62L on CD3⁺CD4+(FIG. 8D) and CD3⁺CD8+(FIG. 8E) T cells in the spleen (top) and peripheral lymph nodes (bottom) (n=5 per group). Representative FACS plots and quantitative analysis of the indicated cell populations and absolute number. Naïve (CD62L^{high}CD44^{low}), effector (CD62L^{low}CD44^{high}) and Central memory (CD62L^{high}CD44high). FIG. 8I, FIG. 8J, WT or TAGLN2-deficient naïve CD8⁺ T cells isolated from the spleen and lymph nodes were activated via CD3/CD28 stimulation for 24 h. Representative histograms and quantitative analysis of CD44 (FIG. 8I) and Ki-67 (FIG. 8J) expression are shown. Data are presented as mean±s.e.m. FIG. 8B-FIG. 8J, Two-tailed unpaired Student's t-test. P<0.05 is considered statistically significant and exact P-values are shown. The ‘n’ values represent biologically independent samples. gMFI, Geometric mean fluorescence intensity.

FIG. 9A-FIG. 9E show that TAGLN2 controls FABP5 surface expression and depends on FABP5 for optimal lipid uptake in activated CD8⁺ T cells. FIG. 9A-FIG. 9C, WT or TAGLN2-deficient naïve CD8⁺ T cells isolated from the spleen and lymph nodes were activated via CD3/CD28 stimulation for 24 h. Representative histograms and quantitative analysis of total FABP5 (FIG. 9A), surface FABP5 (FIG. 9B), and lipid uptake (FIG. 9C) by CD8⁺CD44⁺ T cells of the indicated genotypes are shown (n=3 per genotype). FIG. 9D, FIG. 9E, Naïve CD8⁺ T cells from WT C57BL/6J mice were activated via CD3/CD28 stimulation for 24 h and then Neon-electroporated with either control (Ctrl) or mouse Tagln2 mRNAs, along with non-targeting (control) or Fabp5-specific siRNAs. Experimental scheme and readouts (FIG. 9D). Representative histograms and quantitative analysis of lipid uptake (BODIPY 500/510) in CD8⁺CD44⁺ T cells determined by FACS (n=3 per condition) (FIG. 9E). Data are presented as mean±s.e.m. FIG. 9A-FIG. 9C, Two-tailed unpaired Student's t-test. FIG. 9E, One-way ANOVA with Tukey's multiple comparisons test. P<0.05 is considered statistically significant 10 and exact P-values are shown. The ‘n’ values represent biologically independent samples. gMFI, Geometric mean fluorescence intensity.

FIG. 10A-FIG. 10I show that TAGLN2 downregulation in human and mouse exhausted CD8+ tumor-infiltrating T cells. FIG. 10A, Volcano plot of differentially expressed genes in dysfunctional tumor-specific CD8⁺ T cells (TCR_{TAG}) compared to non-tumor-specific CD8⁺ T cells (TCR_{OT1}) from a mouse autochthonous liver tumor model (GSE126974). Selected differentially expressed genes with an adjusted P values <0.05 and Log₂ Fold Change >1 or −1 are highlighted. FIG. 10B, Relative expression dot plots of the indicated genes in different HGSOC tumor-infiltrating CD8⁺ T cell populations. The color of each dot represents the average normalized expression from high (red) to low (blue). The size of each dot represents the percentage of positive cells for each gene. FIG. 10C, TAGLN2 transcripts were shown in the indicated CD8+ T cell populations from human peripheral blood mononuclear cells (PBMC). The resulting transcript expression values calculated as normalized transcript per million (nTPM), resulting from the internal normalized pipeline (The Human Protein Atlas; proteinatlas.org). FIG. 10D, Overall survival curves for ID8-Defb29/Vegfa OvCa bearing female mice of the indicated genotypes (n=8 per genotype). FIG. 10E, Representative images of hemorrhagic ascites, spleen and omentum from female mice of the indicated genotypes bearing ID8-Defb29/Vegfa OvCa-based HGSC for 28 days. FIG. 10F-FIG. 10I, Expression of TAGLN2 protein (FIG. 10F), frequencies of CD3⁺CD4+ or CD3⁺CD8⁺ T cell (FIG. 10G), frequencies of CD44 and CD62L on CD3⁺CD8+(FIG. 10H) and levels of TOX protein (FIG. 10I) were assessed in the indicated T cell populations from peritoneal lavage, omentum or spleen at day 28 of tumor development. Representative FACS histograms and quantitative analysis are shown (n=8 mice per group). Data are presented as mean±s.e.m. FIG. 10D, Log-rank test for survival. FIG. 10F-FIG. 10I, Two-tailed unpaired Student's t-test. P<0.05 is considered statistically significant and exact P-values are shown. The ‘n’ values represent biologically independent samples. gMFI, Geometric mean fluorescence intensity.

FIG. 11A-FIG. 11G show the putative transcription factor binding sites in the Tagln2 promoter. FIG. 11A, ECR browser analysis of the mouse and human Tagln2 locus is shown. The mouse genomic sequence was used as the base sequence on the x-axis. Schematic representation of the genomic positions of exons (E1-5) and putative binding sites of NF-kB and unfolded protein response (UPR) transcription factors in the Tagln2 promoter regions, mainly in the CNS1 region. Asterisks denote UPR transcription factors. UTR, untranslated region. FIG. 11B, Schematic representation of ER stress sensors and their corresponding downstream transcription factors. FIG. 11C-FIG. 11E, Naïve CD8⁺ T cells isolated from Eif2ak3^{fl/fl} and Eif2ak3^{fl/fl}Vav1^{Cre} mice (FIG. 11C), Atf6^{fl/fl} and Atf6^{fl/fl}Vav1^{Cre} mice (FIG. 11D) or Xbp1^{fl/fl} and Xbp1^{fl/fl}Cd4^{Cre} mice (FIG. 11E) were cultured under the indicated conditions. Expression of the Tagln2 transcript was determined by RT-qPCR, and data were normalized to endogenous levels of Actb in each sample (n=3 per condition and genotype). FIG. 11F, Sequences of mouse Tagln2 promoter from −646 to +138. XBP1s binding sites (Score>10) are marked as BS1 and BS2. Location of ChIP-PCR primers (F/R) is indicated. Figure discloses SEQ ID NO: 8. FIG. 11G, Tagln2 promoter construct used for luciferase reporter assays. FIG. 11C-FIG. 11E, One-way ANOVA with Tukey's multiple comparisons test. P<0.05 is considered to be statistically significant and exact P-values are shown. The ‘n’ values represent biologically independent samples.

FIG. 12A-FIG. 12J show the elevated Tagln2 expression in multiple intratumoral CD4+ and CD8⁺ T cell subsets lacking XBP1s. FIG. 12A, Schematic illustrating sample processing and experimental workflow. FIG. 12B, FACS sorting strategy for scRNA-sequencing. FIG. 12C, UMAP colored by genotype classifications of Xbp1^{fl/fl} (blue, n=4,269 cells) and Xbp1^{fl/fl}Cd4^{Cre} (red, n=3,325 cells). FIG. 12D, UMAP plot visualization of different T cell clusters colored by cell type. FIG. 12E, Heatmap showing the top 10 marker genes of the subclusters. FIG. 12F, FIG. 12G, Volcano plot showing differentially expressed genes (DEGs) with top 10 genes in CD4+(FIG. 12F) or CD8+(FIG. 12G) intratumoral T cell clusters from XBP1s-deficient compared to WT control labeled according to Wilcoxon rank-sum test with Benjamini-Hochberg correction DEG cutoff (min_pct_expression >10%, abs(avg_log 2fc)>0.2, −log 10_pval_adj>2). FIG. 12H, Enriched cellular pathways and functions in XBP1s-deficient CD8⁺ T cells at tumor sites. Z-scores greater than 2 indicate pathways and functions predicted to be significantly increased in XBP1s-deficient CD8⁺ T cells. i, Dot plot analysis showing the expression levels and distribution of seven major cytoskeletal genes (Tagln2, Wipf1, Wasf1, Wasf2, Hcls1, Was and Actr2) identified in each CD4+ and CD8⁺ T cell cluster in (FIG. 12D). FIG. 12J, Expression of previously reported RIDD target genes across all different CD3⁺ T cell clusters identified in (FIG. 12D).

FIG. 13A-FIG. 13H show that selective loss of XBP1s in T cells delays malignant tumor progression and enhances TAGLN2, Ki-67, and CD44 expression in PPNM tumor-infiltrating CD8⁺ T cells. FIG. 13A, Experimental scheme for mice of the indicated genotypes implanted with luciferase-expressing PPNM cancer cells. FIG. 13B, FIG. 13C, Assessment of peritoneal tumor burden over time in mice of the indicated genotypes (FIG. 13B) and quantification of bioluminescent signal for the same mice of the indicated genotype at different time points (n=5 per genotype) (FIG. 13C). FIG. 13D, Overall survival curves for PPNM-bearing female mice of the indicated genotypes (n=9 per genotype). FIG. 13E, FIG. 13F, Representative images of omentum (FIG. 13E) and solid tumors (FIG. 13F) from female mice of the indicated genotypes bearing PPNM-based HGSC for 40 days. Weight of omentum (FIG. 13E) and solid tumors (FIG. 13F) was determined in each group (Xbp1^{fl/fl}, n=5; Xbp1^{fl/fl}Cd4^{Cre} n=8). FIG. 13G, FIG. 13H, Correlation of protein expression levels of TAGLN2 versus either Ki-67 or CD44 in the indicated intratumoral CD8⁺ T cell subsets in omentum (FIG. 13G) and solid tumor (FIG. 13H) from female mice of indicated genotypes bearing PPNM-bearing HGSC for 40 days (Xbp1^{fl/fl}, n=5; Xbp1^{fl/fl}Cd4^{Cre}, n=8). Data are presented as mean±s.e.m. FIG. 13C, One-way ANOVA with Tukey multiple comparisons test. FIG. 13D, Log-rank test for survival. FIG. 13E, FIG. 13F, Two-tailed unpaired Student's t-test. FIG. 13G, FIG. 13H, Spearman's rank correlation test, Spearman coefficient (r) with exact P-value (two-tailed). P<0.05 is considered to be statistically significant and exact P-values are shown. The ‘n’ values represent biologically independent samples. gMFI, Geometric mean fluorescence intensity.

FIG. 14A-FIG. 14O show that ER-stressed CD8⁺ T cells selectively suppress FABP5 surface localization, but ER stressor does not affect the TAGLN2-FABP5 axis in B cells and γδ T cells. FIG. 14A-FIG. 14E, Naïve CD8⁺ T cells from WT C57BL/6J mice were stimulated via CD3/CD28 for 24 h and then Neon-electroporated with either Control (Ctrl) or mouse Tagln2 mRNAs. Electroporated T cells were maintained under CD3/CD28 stimulation for an additional 32 h and then treated with the ER stressor TM (1 μg/ml) for 16 h. Representative FACS histograms and quantitative analysis of TAGLN2 (FIG. 14A), lipid uptake (FIG. 14B), total (left) and surface (right) expression of FABP5 (FIG. 14C), FABP4 (FIG. 14D) and CD36 (FIG. 14E) in CD8⁺CD44⁺ T cells determined by FACS (n=5 per condition). FIG. 14F-FIG. 14J, Pan B cells isolated from Tagln2^{fl/fl} and Tagln2^{fl/fl}Vav1^{Cre} mice were cultured under the indicated conditions (FIG. 14F). Representative FACS histograms and quantitative analysis of TAGLN2 (FIG. 14G), surface FABP5 (FIG. 14H), total FABP5 (FIG. 14I) and lipid uptake (FIG. 14J) in CD19+ B cells. (n=3 per condition). FIG. 14K-FIG. 14O, γδ T cells isolated from Tagln2^{fl/fl} and Tagln2^{fl/fl}Vav1^{Cre} mice were cultured under the indicated conditions (FIG. 14K). Representative FACS histograms and quantitative analysis of TAGLN2 (FIG. 14L), surface FABP5 (FIG. 14M), total FABP5 (FIG. 14N) and lipid uptake (FIG. 14O) in CD3+γ6⁺ T cells. (n=3 per condition). Data are presented as mean±s.e.m. FIG. 14A-FIG. 14E, FIG. 14G-FIG. 14J, FIG. 14L-FIG. 14O, One-way ANOVA with Tukey multiple comparisons test P<0.05 is considered to be statistically significant and exact P-values are shown. The ‘n’ values represent biologically independent samples. gMFI, Geometric mean fluorescence intensity.

FIG. 15A-FIG. 15L show the ER stress responses in CER T cells. FIG. 15A, Main features of the FSH-CER retroviral construct. FIG. 15B, Expression of the follicle-stimulating hormone receptor (FSHR) by PPNM cancer cells was determined by immunoblot analysis in which 3-actin was used as loading control. FIG. 15C, FIG. 15D, CD45.1+CER T cells were isolated from the indicated tumor sites seven days after adoptive transfer into WT C57BL/6J female mice developing PPNM HGSC. Experimental scheme for analysis of isolated CER T cells at tumor locations (FIG. 15C). Xbp1s, Sec61a1, ERdj4, and Tagln2 expression in pre- or post-infusion CD45.1⁺CD3+GFP+CER T cells was determined via qRT-PCR. Data were normalized to Actb (n=12) (FIG. 15D). FIG. 15E-FIG. 15H, Representative FACS histograms and quantitative analysis of TAGLN2 (left) and surface FABP5 (right) protein levels in gated CD45.1⁺CD3+GFP+ T cells isolated from peritoneal lavage (FIG. 15E) or omentum (FIG. 15F) at indicated different days after adoptive transfer into WT C57BL/6J female mice developing PPNM HGSC (n=6-7 per group). Blue bar graph represents the expression of TAGLN2 and surface FABP5 in pre-infusion CER T cells, respectively. Representative FACS plots and quantitative analysis of CD44+IFN-γ⁺ (FIG. 15G) and CD44⁺GZMB⁺(FIG. 15H) frequencies in gated CD45.1⁺CD3+GFP+ T cells isolated from peritoneal lavage or omentum from the same mice described in FIG. 15E and FIG. 15F (n=6-7 per group). FIG. 15I, FIG. 15J, CD8+GFP+sorted CER T cells were stimulated with recombinant chorionic gonadotropin alpha (CG(a) in the absence or presence of TM (FIG. 15I). Xbp1s, Sec61a1, and Tagln2 expression was determined via qRT-PCR. Data were normalized to Actb (n=3 per condition) (FIG. 15J). FIG. 15K, Experimental scheme to assess the effect of ER stress in Mock or FSH-CER transduced CD8⁺ T cells. FIG. 15L, CD8+GFP+sorted Mock transduced T cells were electroporated with the indicated mRNAs and then treated with vehicle or TM for 16 h. T cells were washed to remove TM and then cocultured with PPNM cancer cells at a 1:1 ratio. Cancer cell death was assessed by Annexin V and PI staining by FACS 18 h later. Representative FACS plots and quantitative analysis (n=3-4 per condition). Data are presented as mean±s.e.m. FIG. 15D, Two-tailed paired Student's t-test. FIG. 15E-FIG. 15H, One-way ANOVA with Tukey's multiple comparisons test. FIG. 15J, Two-tailed unpaired Student's t-test. P<0.05 is considered to be statistically significant and exact P-values are shown. The ‘n’ values represent biologically independent samples. gMFI, Geometric mean fluorescence intensity.

FIG. 16A-FIG. 16K show the therapeutic effect of CER-Tagln2 T cells in OvCa. FIG. 16A, FACS-based analysis to assess transduction efficiency using GFP expression as a marker. FIG. 16B, CER or CER-Tagln2 T cells were incubated in the presence or absence of TM for 16 h and TAGLN2 protein expression was measured by FACS. FIG. 16C-FIG. 16G, Adoptive transfer of CER or CER-Tagln2 T cells into MP tumor-bearing WT C57BL/6J female mice. Experimental scheme (FIG. 16C). Expression of the follicle-stimulating hormone receptor (FSHR) by PPNM, MP and BMDC were determined by immunoblot analysis in which 3-actin was used as loading control (FIG. 16D). Representative bioluminescence images of MP tumors over time (FIG. 16E) and quantification of peritoneal tumor burden (FIG. 16F) in the indicated groups (n=8 mice per group). Overall survival rates for the mice (FIG. 16G) described in FIG. 16E and FIG. 16F. The initial number of mice allocated to each experimental group was n=8, but only mice that developed ovarian cancer were included in the survival analysis shown. FIG. 16H-FIG. 16J, WT C57BL/6J female mice were challenged i.p. with PPNM-ovarian cancer cells. After 8 days, mice were treated with either CER or CER-Tagln2 T cells alone or in combination with isotype control or anti-PD-1 antibodies as described in Methods (n=10 mice per group). Representative bioluminescence images of MP tumors over time (FIG. 16I) and overall survival rates for the mice (FIG. 16J) described in FIG. 16I. The initial number of mice allocated to each experimental group was n=10, but only mice that developed ovarian cancer were included in the survival analysis shown. FIG. 16K, Schematic of OvCa-associated CD8⁺ T cells wherein dysregulated ER stress responses induced by the TME disable a major cytoskeletal-mitochondrial axis that guards competent T cell anti-tumor function. ER stress-driven IRE1α-XBP1s signaling blunts TAGLN2, which cooperates with FABP5 to ensure robust lipid uptake, fatty acid oxidation (FAO), mitochondrial respiration, and effector function. TAGLN2-overexpressing CER T cells, redirected to kill OvCa cells, bypass the detrimental effects of the dysregulated ER stress response evoked by the ovarian TME, hence demonstrating enhanced functionality, persistence, and protective activity in hosts with this devastating malignancy. The figure was generated using BioRender. Data are presented as mean±s.e.m. FIG. 16F, One-way ANOVA with Tukey's multiple comparisons test. FIG. 16G, FIG. 16J, Log-rank test for survival. P<0.05 is considered to be statistically significant and exact P-values are shown. The ‘n’ values represent biologically independent samples. gMFI, Geometric mean fluorescence intensity.

### DETAILED DESCRIPTION OF THE INVENTION

Mounting effective immunity against pathogens and tumors relies on the successful metabolic programming of T cells by extracellular fatty acids¹⁻³. During this process, fatty-acid-binding protein 5 (FABP5) imports lipids that fuel mitochondrial respiration and sustain the bioenergetic requirements of protective CD8⁺ T cells^{4,5}. Importantly, however, the mechanisms governing this crucial immunometabolic axis remain unexplored. Here we report that the cytoskeletal organizer Transgelin 2 (TAGLN2) is necessary for optimal CD8⁺ T cell fatty acid uptake, mitochondrial respiration, and anti-cancer function. We found that TAGLN2 interacts with FABP5, enabling the surface localization of this lipid importer on activated CD8⁺ T cells. Analysis of ovarian cancer specimens revealed that endoplasmic reticulum (ER) stress responses elicited by the tumor microenvironment repress TAGLN2 in infiltrating CD8⁺ T cells, enforcing their dysfunctional state. Restoring TAGLN2 expression in ER-stressed CD8⁺ T cells bolstered their lipid uptake, mitochondrial respiration, and cytotoxic capacity. Accordingly, chimeric antigen receptor T cells overexpressing TAGLN2 bypassed the detrimental effects of tumor-induced ER stress and demonstrated superior therapeutic efficacy in mice with metastatic ovarian cancer. Our study unveils the role of cytoskeletal TAGLN2 in T cell lipid metabolism and highlights the potential to enhance cellular immunotherapy in solid malignancies by preserving the TAGLN2-FABP5 axis.

### Definitions

The articles “a” and “an” are used herein to refer to one or to more than one (e.g., to at least one) of the grammatical object of the article. By way of example, “an element” means one element or more than one element.

As used herein, the term “administering” means providing a pharmaceutical agent or composition to a subject, and includes, but is not limited to, administering by a medical professional and self-administering. Such an agent can contain, for example, a cell expressing a CAR provided herein.

The term “amino acid” is intended to embrace all molecules, whether natural or synthetic, which include both an amino functionality and an acid functionality and capable of being included in a polymer of naturally-occurring amino acids. Example amino acids include naturally-occurring amino acids; analogs, derivatives and congeners thereof; amino acid analogs having variant side chains; and all stereoisomers of any of the foregoing.

The term “binding” or “interacting” refers to an association, which may be a stable association, between two molecules, e.g., between a peptide and a binding partner or agent, e.g., small molecule, due to, for example, electrostatic, hydrophobic, ionic and/or hydrogen-bond interactions under physiological conditions.

As used herein, the term “cancer” includes, but is not limited to, solid tumors and blood borne tumors. The term cancer includes, but is not limited to, diseases of the skin, tissues, organs, bone, cartilage, blood, and vessels, including the cervix, anus, vagina, vulva, penis, tongue base, larynx, and tonsil. The term “cancer” further encompasses primary and metastatic cancers.

The term “chimeric antigen receptor” (CAR) refers to molecules that combine a binding domain against a component present on the target cell, for example an antibody-based specificity for a desired antigen (e.g., a tumor antigen) with an immune cell-activating intracellular domain to generate a chimeric protein. Generally, CARs comprise an extracellular single chain antigen-binding domain (e.g., an scFv) fused to the intracellular signaling domain. In some embodiments, the CAR described herein is a chimeric endocrine receptor (CER). In some embodiments, the CER described herein comprises: a) the β subunit of the follicle-stimulating hormone (FSHβ), b) a spacer (e.g., a glycine/serine spacer), c) a transmembrane domain (e.g., a CD8α transmembrane domain), and d) an intracellular domain (e.g., a 4-1BB-CD3ζ (BBZ) intracellular domain (ICD)).

The term “epitope” means a protein determinant capable of specific binding to an antibody or immune cell (e.g., T cell). Epitopes usually include chemically active surface groupings of molecules such as amino acids or sugar side chains. Certain epitopes can be defined by a particular sequence of amino acids to which a CAR or antibody is capable of binding.

“Gene construct” refers to a nucleic acid, such as a vector, plasmid, viral genome or the like which includes a “coding sequence” for a polypeptide or which can otherwise transcribe to a biologically active RNA (e.g., antisense, decoy, ribozyme, etc.), may be transfected into cells, e.g., mammalian cells, and may cause expression of the coding sequence in cells transfected with the construct. The gene construct may include one or more regulatory elements operably linked to the coding sequence, as well as intronic sequences, polyadenylation sites, origins of replication, marker genes, etc.

The terms “ligand-binding domain” and “antigen-binding domain” are used interchangeably herein, and refer to that portion of a chimeric antigen receptor that binds specifically to a predetermined antigen.

The term “linker” refers to a molecule or group of molecules connecting two compounds, such as two polypeptides. The linker may be comprised of a single linking molecule or may comprise a linking molecule and a spacer molecule, intended to separate the linking molecule and a compound by a specific distance.

The term “operably linked to” refers to the functional relationship of a nucleic acid with another nucleic acid sequence. Promoters, enhancers, transcriptional and translational stop sites, and other signal sequences are examples of nucleic acid sequences operably linked to other sequences. For example, operable linkage of DNA to a transcriptional control element refers to the physical and functional relationship between the DNA and promoter such that the transcription of such DNA is initiated from the promoter by an RNA polymerase that specifically recognizes, binds to and transcribes the DNA.

As used herein, the phrase “pharmaceutically acceptable” refers to those agents, compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the phrase “pharmaceutically-acceptable carrier” means a pharmaceutically-acceptable material, composition or vehicle, such as a liquid or solid filler, diluent, excipient, or solvent encapsulating material, involved in carrying or transporting an agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; and (22) other non-toxic compatible substances employed in pharmaceutical formulations.

The terms “polynucleotide”, and “nucleic acid” are used interchangeably. They refer to a natural or synthetic molecule, or some combination thereof, comprising a single nucleotide or two or more nucleotides linked by a phosphate group at the 3′ position of one nucleotide to the 5′ end of another nucleotide. The polymeric form of nucleotides is not limited by length and can comprise either deoxyribonucleotides or ribonucleotides, or analogs thereof. Polynucleotides may have any three-dimensional structure, and may perform any function. The following are non-limiting examples of polynucleotides: coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. A polynucleotide may be further modified, such as by conjugation with a labeling component. In all nucleic acid sequences provided herein, U nucleotides are interchangeable with T nucleotides. The polynucleotide is not necessarily associated with the cell in which the nucleic acid is found in nature, and/or operably linked to a polynucleotide to which it is linked in nature.

The term “precancerous lesions” or “precancerous condition” refers to atypical cells and/or tissues that are associated with an increased risk of cancer. The term “precancerous lesions” may refer, for example, to dysplasia, benign neoplasia, or carcinoma in situ.

As used herein, a therapeutic that “prevents” a condition refers to a compound that, when administered to a statistical sample prior to the onset of the disorder or condition, reduces the occurrence of the disorder or condition in the treated sample relative to an untreated control sample, or delays the onset or reduces the severity of one or more symptoms of the disorder or condition relative to the untreated control sample.

An “intracellular signaling region” of a CAR, as used herein, is the part of the chimeric antigen receptor protein that is located within the cell that is responsible for intracellular signaling following the binding of an extracellular antigen binding domain to the target. In some embodiments, the intracellular signal region can include multiple intracellular domains (ICDs) that may each convey a separate intracellular signal. In some cases, the intracellular signaling region comprises a pair of an intracellular domain 1 (ICD1) and an intracellular domain 2 (ICD2) listed in Tables 1-3.

The term “specifically binds” or “specific binding”, as used herein, when referring to a polypeptide (including CAR polypeptides) refers to a binding reaction which is determinative of the presence of the protein or polypeptide or receptor in a heterogeneous population of proteins and other biologics. Thus, under designated conditions (e.g. immunoassay conditions in the case of an antibody), a specified ligand or antibody “specifically binds” to its particular “target” (e.g. an antibody specifically binds to an endothelial antigen) when it does not bind in a significant amount to other proteins present in the sample or to other proteins to which the ligand or antibody may come in contact in an organism. Generally, a first molecule that “specifically binds” a second molecule has an affinity constant (Ka) greater than about 10⁵ M⁻¹ (e.g., 10⁶ M⁻¹, 10⁷ M⁻¹, 10⁸ M⁻¹, 10⁹ M⁻¹, 10¹⁰ M⁻¹, 10¹¹ M⁻¹, and 10¹² M⁻¹ or more) with that second molecule. For example, in the case of the ability of a PIG-specific CAR to bind to a peptide presented on an MHC (e.g., class I MHC or class II MHC); typically, a CAR specifically binds to its peptide/MHC with an affinity of at least a KD of about 10⁻⁴ M or less, and binds to the predetermined antigen/binding partner with an affinity (as expressed by KD) that is at least 10 fold less, at least 100 fold less or at least 1000 fold less than its affinity for binding to a non-specific and unrelated peptide/MHC complex (e.g., one comprising a BSA peptide or a casein peptide).

As used herein, the term “subject” means a human or non-human animal selected for treatment or therapy.

The terms “transformation”, “transfection”, or “transduction” mean the introduction of a nucleic acid, e.g., an expression vector, into a recipient cell (e.g., a mammalian cell) including introduction of a nucleic acid to the chromosomal DNA of said cell.

As used herein, the term “treatment” refers to clinical intervention designed to alter the natural course of the individual being treated during the course of clinical pathology. Desirable effects of treatment include decreasing the rate of progression, ameliorating or palliating the pathological state, and remission or improved prognosis of a particular disease, disorder, or condition. An individual is successfully “treated,” for example, if one or more symptoms associated with a particular disease, disorder, or condition are mitigated or eliminated.

The term “vector” refers to the means by which a nucleic acid can be propagated and/or transferred between organisms, cells, or cellular components. Vectors include plasmids, viruses, bacteriophage, pro-viruses, phagemids, transposons, and artificial chromosomes, and the like, to which the nucleic acid has been linked, and may or may not be able to replicate autonomously or integrate into a chromosome of a host cell. Such vectors may include any vector, (e.g., a plasmid, cosmid or phage chromosome) containing a gene construct in a form suitable for expression by a cell (e.g., linked to a transcriptional control element).

In certain embodiments, agents may be used alone or conjointly administered with another type of therapeutic agent. As used herein, the phrase “conjoint administration” or “administered conjointly” refers to any form of administration of two or more different therapeutic agents such that the second agent is administered while the previously administered therapeutic agent is still effective in the body (e.g., the two agents are simultaneously effective in the subject, which may include synergistic effects of the two agents). For example, the different therapeutic agents can be administered either in the same formulation or in separate formulations, either concomitantly or sequentially. In certain embodiments, the different therapeutic agents can be administered within about one hour, about 12 hours, about 24 hours, about 36 hours, about 48 hours, about 72 hours, or about a week of one another. Thus, a subject who receives such treatment can benefit from a combined effect of different therapeutic agents.

Transgelin-2 (TAGLN2), also known as HA1756, encode a protein that is similar to the protein transgelin, which is one of the earliest markers of differentiated smooth muscle. The specific function of the Transgelin-2 (TAGLN2) protein has not yet been determined, although it is thought to be a tumor suppressor. Multiple transcript variants encoding different isoforms have been found for this gene. For example, at least three human Transgelin-2 (TAGLN2) transcript variants are known. The transcript variant (1) (NM_001277224.2), represents the longest transcript and encodes the longer isoform (a) (NP_001264153.1). The transcript variant (2) (NM_003564.3), uses an alternate first exon and begins translation at a downstream AUG compared to variant 1. The resulting isoform (b) (NP_003555.1) is shorter at the N-terminus compared to isoform a. The transcript variant (3) (NM_001277223.2) uses an alternate first exon and begins translation at a downstream AUG compared to variant 1. The resulting isoform (b) (NP_001264152.1) is shorter at the N-terminus compared to isoform a. Variants 2 and 3 both encode the same isoform (b). Nucleic acid and polypeptide sequences of TAGLN2 orthologs in organisms other than humans are well known and include, for example, chimpanzee TAGLN2 (XM_003308536.7 and XP_003308584.1; and XM_003308536.7 and XP_003308584.1), Rhesus monkey TAGLN2 (NM_001265900.1 and NP_001252829.1), dog TAGLN2 (XM_038650801.1 and XP_038506729.1), cattle TAGLN2 (NM_001013599.1 and NP_001013617.1), rat TAGLN2 (XM_039090801.2 and XP_038946729.1; XM_006250295.4 and XP_006250357.1; and XM_063272375.1 and XP_063128445.1), and mouse TAGLN2 (NM_178598.2 and NP_848713.1).

TABLE 4A
Exemplary cDNA sequences of Transgelin-2 (TAGLN2)

```
SEQ ID NO: 1
cDNA Sequence of Human TAGLN2 transcript variant 1
(NM_001277224.2, CDS region from position 265-927)
      1 cttctgctcc tgactctcgg tggtgagggg ctgccctacg gtggttccct taattgtggg
    61 ccgcccccca tccttgctgc tggctgtggc acctcgctct gtgggctggt gccctcactg
  121 tgctgctctt tgagaacact ccctgtccca aatctgagtt gggaagttct cctgtgttcc
  181 atctgcacgg tggatggaga gtccctttgt cttcccaatt aaacacccta gccaagaacc
  241 ctgaagtcct ccctgccccc acccatgtcc gccttttctt tggctttggc tttggtgagc
  301 tccccgcagc caccgccacc cattggaatg gccaacaggg gacctgcata tggcctgagc
  361 cgggaggtgc agcagaagat tgagaaacaa tatgatgcag atctggagca gatcctgatc
  421 cagtggatca ccacccagtg ccgaaaggat gtgggccggc cccagcctgg acgcgagaac
  481 ttccagaact ggctcaagga tggcacggtg ctatgtgagc tcattaatgc actgtacccc
  541 gaggggcagg ccccagtaaa gaagatccag gcctccacca tggccttcaa gcagatggag
  601 cagatctctc agttcctgca agcagctgag cgctatggca ttaacaccac tgacatcttc
  661 caaactgtgg acctctggga aggaaagaac atggcctgtg tgcagcggac gctgatgaat
  721 ctgggtgggc tggcagtagc ccgagatgat gggctcttct ctggggatcc caactggttc
  781 cctaagaaat ccaaggagaa tcctcggaac ttctcggata accagctgca agagggcaag
  841 aacgtgatcg ggttacagat gggcaccaac cgcggggcgt ctcaggcagg catgactggc
  901 tacgggatgc cacgccagat cctctgatcc caccccaggc cttgcccctg ccctcccacg
  961 aatggttaat atatatgtag atatatattt tagcagtgac attcccagag agccccagag
1021 ctctcaagct cctttctgtc agggtggggg gttcagcctg tcctgtcacc tctgaggtgc
1081 ctgctggcat cctctccccc atgcttacta atacattccc ttccccatag ccatcaaaac
1141 tggaccaact ggcctcttcc tttcccctgg gaccaaaatt taggggcctc agtccctcac
1201 cgccatgccc tggcctattc tgtctctcct tcttccccct ggcctgttct gtctctgagc
1261 tctgtgtcct ccgttcattc catggctggg agtcactgat gctgcctctg ccttctgatg
1321 ctggactggc cttgcttcta caagtatgct tctcccacag ctgtggctgc aggaacttaa
1381 tttataggga ggagcctgtg gcagctgctg ccccagccac agctgcactg actgtgctca
1441 ccacacatct ggggcagcct tccctggcag gggccctcgt ggcttctcat tttccattcc
1501 cttcactgtg gctaaggggt ggggtgaggg gatggagagg gagggctgcc taccatggtc
1561 tggggcttga ggaagatgag tttgttgatt taaataaaga atttgtcatt tttgaa
SEQ ID NO: 2
cDNA Sequence of Human TAGLN2 transcript variant 2
(NM_003564.3, CDS region from position 87-686)
      1 acttgcagct gcagcccttg ccttgagtca gtgcgccgct ctccagcccg cttgaacgct
    61 ccccgcagcc accgccaccc attggaatgg ccaacagggg acctgcatat ggcctgagcc
  121 gggaggtgca gcagaagatt gagaaacaat atgatgcaga tctggagcag atcctgatcc
  181 agtggatcac cacccagtgc cgaaaggatg tgggccggcc ccagcctgga cgcgagaact
  241 tccagaactg gctcaaggat ggcacggtgc tatgtgagct cattaatgca ctgtaccccg
  301 aggggcaggc cccagtaaag aagatccagg cctccaccat ggccttcaag cagatggagc
  361 agatctctca gttcctgcaa gcagctgagc gctatggcat taacaccact gacatcttcc
  421 aaactgtgga cctctgggaa ggaaagaaca tggcctgtgt gcagcggacg ctgatgaatc
  481 tgggtgggct ggcagtagcc cgagatgatg ggctcttctc tggggatccc aactggttcc
  541 ctaagaaatc caaggagaat cctcggaact tctcggataa ccagctgcaa gagggcaaga
  601 acgtgatcgg gttacagatg ggcaccaacc gcggggcgtc tcaggcaggc atgactggct
  661 acgggatgcc acgccagatc ctctgatccc accccaggcc ttgcccctgc cctcccacga
  721 atggttaata tatatgtaga tatatatttt agcagtgaca ttcccagaga gccccagagc
  781 tctcaagctc ctttctgtca gggtgggggg ttcagcctgt cctgtcacct ctgaggtgcc
  841 tgctggcatc ctctccccca tgcttactaa tacattccct tccccatagc catcaaaact
  901 ggaccaactg gcctcttcct ttcccctggg accaaaattt aggggcctca gtccctcacc
  961 gccatgccct ggcctattct gtctctcctt cttccccctg gcctgttctg tctctgagct
1021 ctgtgtcctc cgttcattcc atggctggga gtcactgatg ctgcctctgc cttctgatgc
1081 tggactggcc ttgcttctac aagtatgctt ctcccacagc tgtggctgca ggaacttaat
1141 ttatagggag gagcctgtgg cagctgctgc cccagccaca gctgcactga ctgtgctcac
1201 cacacatctg gggcagcctt ccctggcagg ggccctcgtg gcttctcatt ttccattccc
1261 ttcactgtgg ctaaggggtg gggtgagggg atggagaggg agggctgcct accatggtct
1321 ggggcttgag gaagatgagt ttgttgattt aaataaagaa tttgtcattt ttgaa
SEQ ID NO: 3
cDNA Sequence of Human TAGLN2 transcript variant 3
(NM_001277223.2, CDS region from position 131-730)
      1 ggcggctggg aggccggacc tgcaggaccg gaggtggact gccgcctccc cccgcgccct
    61 gtgcctcctg gggctacctg aagccggtgt cctgggagat ctctccccgc agccaccgcc
  121 acccattgga atggccaaca ggggacctgc atatggcctg agccgggagg tgcagcagaa
  181 gattgagaaa caatatgatg cagatctgga gcagatcctg atccagtgga tcaccaccca
  241 gtgccgaaag gatgtgggcc ggccccagcc tggacgcgag aacttccaga actggctcaa
  301 ggatggcacg gtgctatgtg agctcattaa tgcactgtac cccgaggggc aggccccagt
  361 aaagaagatc caggcctcca ccatggcctt caagcagatg gagcagatct ctcagttcct
  421 gcaagcagct gagcgctatg gcattaacac cactgacatc ttccaaactg tggacctctg
  481 ggaaggaaag aacatggcct gtgtgcagcg gacgctgatg aatctgggtg ggctggcagt
  541 agcccgagat gatgggctct tctctgggga tcccaactgg ttccctaaga aatccaagga
  601 gaatcctcgg aacttctcgg ataaccagct gcaagagggc aagaacgtga tcgggttaca
  661 gatgggcacc aaccgcgggg cgtctcaggc aggcatgact ggctacggga tgccacgcca
  721 gatcctctga tcccacccca ggccttgccc ctgccctccc acgaatggtt aatatatatg
  781 tagatatata ttttagcagt gacattccca gagagcccca gagctctcaa gctcctttct
  841 gtcagggtgg ggggttcagc ctgtcctgtc acctctgagg tgcctgctgg catcctctcc
  901 cccatgctta ctaatacatt cccttcccca tagccatcaa aactggacca actggcctct
  961 tcctttcccc tgggaccaaa atttaggggc ctcagtccct caccgccatg ccctggccta
1021 ttctgtctct ccttcttccc cctggcctgt tctgtctctg agctctgtgt cctccgttca
1081 ttccatggct gggagtcact gatgctgcct ctgccttctg atgctggact ggccttgctt
1141 ctacaagtat gcttctccca cagctgtggc tgcaggaact taatttatag ggaggagcct
1201 gtggcagctg ctgccccagc cacagctgca ctgactgtgc tcaccacaca tctggggcag
1261 ccttccctgg caggggccct cgtggcttct cattttccat tcccttcact gtggctaagg
1321 ggtggggtga ggggatggag agggagggct gcctaccatg gtctggggct tgaggaagat
1381 gagtttgttg atttaaataa agaatttgtc atttttgaa
SEQ ID NO: 6
cDNA Sequence of Mouse TAGLN2 transcript (NM_178598.2, CDS
region from position 116-715)
      1 gaaaactcca agcccggccg ggtcttgagc tccactcgcc gctgcagccc ctgtcgtgcg
    61 tgcgctctca tccagccctc ttggacgctc tttgccatca ccacagctgc tcagaatggc
  121 caacagggga ccttcctacg gcctgagccg agaggtgcag cagaagattg agaagcagta
  181 cgacgcggat ctggagcaga tcctcatcca gtggatcacc actcagtgcc gtgaggacgt
  241 gggccagccc cagcctggcc gtgagaactt ccagaagtgg ctcaaggacg gcacggttct
  301 gtgcaagctt attaattcac tgtatcctga ggggcaggcc ccagtaaaga agatccaggc
  361 ctcttcgatg gccttcaagc agatggagca gatctcccag ttcctgcagg cagccgagcg
  421 ctatggcatt aacaccacgg acatcttcca gactgtggat ctctgggaag gaaagaacat
  481 ggcttgtgtg cagcggacac taatgaacct gggtgggctg gcagtagcca gggacgatgg
  541 gctcttctct ggggatccca actggtttcc taagaaatcc aaggagaacc ctcggaactt
  601 ctcggacaac cagttgcaag agggcaagaa cgtgattggg ttgcagatgg gcaccaaccg
  661 tggagcatct caggccggca tgaccggcta tgggatgcca cggcagatcc tctgatcata
  721 ctctctctcc ttcccctgcc ctccatgaat ggttaatata tatgtatata tatgttttag
  781 cagacattcc ctgagagccc ctggattgct gaactcccct ctgccagggt ccaggccagc
  841 ctatcttgtc accactggca gggcctgata attgcctctc tctctctctc tctttctctc
  901 tctctctctc tctctctctc tctctctctc tctctgggct tactaatgca ttccttcccc
  961 cacaaccatc aaaactggac caacaaaaac cctgggacca aagttgcctc cccacagcat
1021 ctttcctgct ttcctgatct cttcttttag tccatccctt ggctaggagt cagagattct
1081 gccccatggc ctgatgctgc accgaccctt ccttctacaa ggaggcctct cctacagctg
1141 tggctgcagg gacttaattt atagggaggg gcctgtggct gtcactccag ccacagctgg
1201 gctgtactta ccacacgtct gggcagcttt ccctagcaga ggctctttgg cttctttctt
1261 tccattcctc tctcactgtg gctaaggggt ggagcagagg taggacggct gcccaccatg
1321 ctctggggct tgacgaacac gagtttgctg attttaaata aaaagatctc attttgtttt
1381 gc
```

TABLE 4B
Exemplary amino acid sequences of Transgelin-2 (TAGLN2)

SEQ ID NO: 4 Amino Acid Sequence of Human TAGLN2 Isoform A (NP_001264153.1)

| | |
|---|---|
| 1 | msafslalal vsspqppppi gmanrgpayg lsrevqqkie kqydadleqi liqwittqcr |
| 61 | kdvgrpqpgr enfqnwlkdg tvlcelinal ypegqapvkk iqastmafkq meqisqflqa |
| 121 | aeryginttd ifqtvdlweg knmacvqrtl mnlgglavar ddglfsgdpn wfpkkskenp |
| 181 | rnfsdnqlqe gknviglqmg tnrgasqagm tgygmprqil |

SEQ ID NO: 5 Amino Acid Sequence of Human TAGLN2 Isoform B (NP_003555.1 and
NP_001264152.1)

| | |
|---|---|
| 1 | manrgpaygl srevqqkiek qydadleqil iqwittqcrk dvgrpqpgre nfqnwlkdgt |
| 61 | vlcelinaly pegqapvkki qastmafkqm eqisqflqaa eryginttdi fqtvdlwegk |
| 121 | nmacvqrtlm nlgglavard dglfsgdpnw fpkkskenpr nfsdnqlqeg knviglqmgt |
| 181 | nrgasqagmt gygmprqil |

SEQ ID NO: 7 Amino Acid Sequence of Mouse TAGLN2 (NP_848713.1)

| | |
|---|---|
| 1 | manrgpsygl srevqqkiek qydadleqil iqwittqcre dvgqpqpgre nfqkwlkdgt |
| 61 | vlcklinsly pegqapvkki qassmafkqm eqisqflqaa eryginttdi fqtvdlwegk |
| 121 | nmacvqrtlm nlgglavard dglfsgdpnw fpkkskenpr nfsdnqlqeg knviglqmgt |
| 181 | nrgasqagmt gygmprqil |

### Agents and Compositions

In some embodiments, provided herein are agents that increase expression, level, and/or activity of Transgelin-2 (TAGLN2) in T cells. In some embodiments, the agent is a small molecule compound, a RNA, a DNA, or a protein. In some embodiments, the agent is a nucleic acid that encodes TAGLN2 or a recombinant TAGLN2 protein.

a. Isolated Nucleic Acids

One aspect of the present invention pertains to methods utilizing isolated nucleic acid molecules that encode a TAGLN2 protein, or biologically active portions thereof. As used herein, the term “nucleic acid molecule” is intended to include DNA molecules (i.e., cDNA or genomic DNA) and RNA molecules (i.e., mRNA) and analogs of the DNA or RNA generated using nucleotide analogs. The nucleic acid molecule can be single-stranded or double-stranded, but preferably is double-stranded DNA. An “isolated” nucleic acid molecule is one which is separated from other nucleic acid molecules which are present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule that encodes a TAGLN2 protein, or biologically active portions thereof, can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or chemical precursors or other chemicals when chemically synthesized.

A nucleic acid molecule that encodes a TAGLN2 protein, or biologically active portions thereof, of the present invention, e.g., a nucleic acid molecule having the nucleotide sequence of SEQ ID NOS: 1, 2, 3, 6, or a nucleotide sequence which is at least about 50%, preferably at least about 60%, more preferably at least about 70%, yet more preferably at least about 80%, still more preferably at least about 90%, and most preferably at least about 95% or more (e.g., about 98%) homologous to the nucleotide sequence shown in SEQ ID NOS: 1, 2, 3, 6, or a portion thereof (i.e., 100, 200, 300, 400, 500, or more nucleotides), can be isolated using standard molecular biology techniques and the sequence information provided herein. For example, a TAGLN2 cDNA can be isolated from a human T cell line using all or portion of SEQ ID NOS: 1, 2, 3, 6, or fragment thereof, as a hybridization probe and standard hybridization techniques (i.e., as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, NY, 1989). Moreover, a nucleic acid molecule encompassing all or a portion of SEQ ID NOS: 1, 2, 3, 6, or a nucleotide sequence which is at least about 50%, preferably at least about 60%, more preferably at least about 70%, yet more preferably at least about 80%, still more preferably at least about 90%, and most preferably at least about 95% or more homologous to the nucleotide sequence shown in SEQ ID NOS: 1, 2, 3, 6, or fragment thereof, can be isolated by the polymerase chain reaction using oligonucleotide primers designed based upon the sequence of SEQ ID NOS: 1, 2, 3, 6, or fragment thereof, or the homologous nucleotide sequence. For example, mRNA can be isolated from T cells and cDNA can be prepared using reverse transcriptase (i.e., Moloney MLV reverse transcriptase, available from Gibco/BRL, Bethesda, MD; or AMV reverse transcriptase, available from Seikagaku America, Inc., St. Petersburg, FL). Synthetic oligonucleotide primers for PCR amplification can be designed based upon the nucleotide sequence shown in SEQ ID NOS: 1, 2, 3, 6, or fragment thereof, or to the homologous nucleotide sequence. A nucleic acid disclosed herein can be amplified using cDNA or, alternatively, genomic DNA, as a template and appropriate oligonucleotide primers according to standard PCR amplification techniques. The nucleic acid so amplified or generated can be cloned into an appropriate vector and characterized by DNA sequence analysis. Furthermore, oligonucleotides corresponding to a TAGLN2 nucleotide sequence can be prepared by standard synthetic techniques, i.e., using an automated DNA synthesizer.

Nucleic acid molecules encoding other TAGLN2s and thus having a nucleotide sequence which differs from the sequences of SEQ ID NOS: 1, 2, 3, 6, or fragment thereof, are contemplated. Moreover, nucleic acid molecules encoding TAGLN2s from different species, and thus which have a nucleotide sequence which differs from the sequences of SEQ ID NOS: 1, 2, 3, 6 are also intended to be within the scope of the present invention. For example, chimpanzee or monkey TAGLN2 cDNA can be identified based on the nucleotide sequence of a human and/or mouse TAGLN2.

In one embodiment, the nucleic acid molecule(s) of the invention encodes a protein or portion thereof which includes an amino acid sequence which is sufficiently homologous to an amino acid sequence of SEQ ID NOS: 4, 5, 7, or fragment thereof, such that the protein or portion thereof reduces the number of viable or proliferating cells in the cancer, and/or reduces the volume or size of a tumor comprising the cancer cells. Methods and assays for measuring each such biological activity are well-known in the art and representative, non-limiting embodiments are described in the Examples below and Definitions above.

As used herein, the language “sufficiently homologous” refers to proteins or portions thereof which have amino acid sequences which include a minimum number of identical or equivalent (e.g., an amino acid residue which has a similar side chain as an amino acid residue in SEQ ID NOS: 4, 5, 7, or fragment thereof) amino acid residues to an amino acid sequence of SEQ ID NOS: 4, 5, 7, or fragment thereof, such that the protein or portion thereof reduces the number of viable or proliferating cells in the cancer, and/or reduces the volume or size of a tumor comprising the cancer cells.

In another embodiment, the protein is at least about 50%, preferably at least about 60%, more preferably at least about 70%, 75%, 80%, 85%, 90%, 91%, 92%, 93%, 94%, 95%, 96%, 97%, 98%, 99% or more homologous to the entire amino acid sequence of SEQ ID NOS: 4, 5, 7, or a fragment thereof.

Portions of proteins encoded by the TAGLN2 nucleic acid molecule of the present invention are preferably biologically active portions of the TAGLN2 polypeptide. As used herein, the term “biologically active portion of TAGLN2 polypeptide” is intended to include a portion, e.g., a domain/motif, of TAGLN2 polypeptide that has one or more of the biological activities of the full-length TAGLN2 polypeptide, respectively.

Standard binding assays, e.g., immunoprecipitations and yeast two-hybrid assays, as described herein, or functional assays, e.g., RNAi or overexpression experiments, can be performed to determine the ability of a TAGLN2 polypeptide or a biologically active fragment thereof to maintain a biological activity of the full-length TAGLN2 polypeptide.

The invention further encompasses nucleic acid molecules that differ from the nucleotide sequence shown in SEQ ID NOS: 1, 2, 3, 6, or fragment thereof due to degeneracy of the genetic code and thus encode the same TAGLN2 polypeptide as that encoded by the nucleotide sequence shown in SEQ ID NOS: 1, 2, 3, 6, or fragment thereof. In another embodiment, an isolated nucleic acid molecule of the invention has a nucleotide sequence encoding a protein having an amino acid sequence shown in SEQ ID NOS: 4, 5, 7, or fragment thereof, or a protein having an amino acid sequence which is at least about 70%, 75%, 80%, 85%, 90%, 91%, 92%, 93%, 94%, 95%, 96%, 97%, 98%, 99% or more homologous to the amino acid sequence of SEQ ID NOS: 4, 5, 7, or fragment thereof, or differs by at least 1, 2, 3, 6, 5 or 10 amino acids but not more than 30, 20, 15 amino acids from SEQ ID NOS: 4, 5, 7. In another embodiment, a nucleic acid encoding a TAGLN2 polypeptide consists of nucleic acid sequence encoding a portion of a full-length TAGLN2 polypeptide fragment of interest that is less than 195, 190, 185, 180, 175, 170, 165, 160, 155, 150, 145, 140, 135, 130, 125, 120, 115, 110, 105, 100, 95, 90, 85, 80, 75, or 70 amino acids in length.

It will be appreciated by those skilled in the art that DNA sequence polymorphisms that lead to changes in the amino acid sequences of TAGLN2 polypeptides may exist within a population (e.g., a mammalian population, e.g., a human population). Such genetic polymorphism in the TAGLN2 gene may exist among individuals within a population due to natural allelic variation. As used herein, the terms “gene” and “recombinant gene” refer to nucleic acid molecules comprising an open reading frame encoding a TAGLN2 protein, preferably a mammalian, e.g., human, TAGLN2 protein. Such natural allelic variations can typically result in 1-5% variance in the nucleotide sequence of the TAGLN2 gene. Any and all such nucleotide variations and resulting amino acid polymorphisms in TAGLN2 that are the result of natural allelic variation and that do not alter the functional activity of TAGLN2 are intended to be within the scope of the invention. Moreover, nucleic acid molecules encoding TAGLN2 proteins from other species, and thus which have a nucleotide sequence which differs from the sequences of SEQ ID NOS: 1, 2, 3, 6, are intended to be within the scope of the invention. Nucleic acid molecules corresponding to natural allelic variants and homologues of the human or mouse TAGLN2 cDNAs of the present invention can be isolated based on their homology to the human or mouse TAGLN2 nucleic acid sequences disclosed herein using the human or mouse cDNA, or a portion thereof, as a hybridization probe according to standard hybridization techniques under stringent hybridization conditions (as described herein).

In addition to naturally-occurring allelic variants of the TAGLN2 sequence that may exist in the population, the skilled artisan will further appreciate that changes can be introduced by mutation into the nucleotide sequence of SEQ ID NOS: 1, 2, 3, 6, or fragment thereof, thereby leading to changes in the amino acid sequence of the encoded TAGLN2 polypeptide, without altering the functional ability of the TAGLN2 polypeptide. For example, nucleotide substitutions leading to amino acid substitutions at “non-essential” amino acid residues can be made in the sequence of SEQ ID NOS: 4, 5, 7, or fragment thereof. A “non-essential” amino acid residue is a residue that can be altered from the sequence of TAGLN2 polypeptide (e.g., the sequence of SEQ ID NOS: 4, 5, 7, or fragment thereof) without significantly altering the activity of TAGLN2 polypeptide, whereas an “essential” amino acid residue is required for TAGLN2 polypeptide activity. Other amino acid residues, however, (e.g., those that are not conserved or only semi-conserved between mouse and human) may not be essential for activity and thus are likely to be amenable to alteration without altering TAGLN2 polypeptide activity. Furthermore, amino acid residues that are essential for TAGLN2 polypeptide functions related to cancer cell viability and/or proliferation, but not essential for other TAGLN2 polypeptide functions, are likely to be amenable to alteration.

Accordingly, another aspect of the present invention pertains to nucleic acid molecules encoding TAGLN2 polypeptides that contain changes in amino acid residues that are not essential for TAGLN2 polypeptide activity. Such TAGLN2 polypeptides differ in amino acid sequence from SEQ ID NOS: 4, 5, 7, or fragment thereof, yet retain at least one of the TAGLN2 polypeptide activities described herein. In one embodiment, the isolated nucleic acid molecule comprises a nucleotide sequence encoding a protein, wherein the protein lacks one or more TAGLN2 polypeptide domains. As stated in the Definitions section, the structure-function relationship of TAGLN2 polypeptide is known such that the ordinarily skilled artisan readily understands the regions that may be mutated or otherwise altered while preserving at least one biological activity of TAGLN2 polypeptide.

“Sequence identity or homology”, as used herein, refers to the sequence similarity between two polypeptide molecules or between two nucleic acid molecules. When a position in both of the two compared sequences is occupied by the same base or amino acid monomer subunit, e.g., if a position in each of two DNA molecules is occupied by adenine, then the molecules are homologous or sequence identical at that position. The percent of homology or sequence identity between two sequences is a function of the number of matching or homologous identical positions shared by the two sequences divided by the number of positions compared x 100. For example, if 6 of 10, of the positions in two sequences are the same then the two sequences are 60% homologous or have 60% sequence identity. By way of example, the DNA sequences ATTGCC and TATGGC share 50% homology or sequence identity. Generally, a comparison is made when two sequences are aligned to give maximum homology. Unless otherwise specified “loop out regions”, e.g., those arising from deletions or insertions in one of the sequences are counted as mismatches.

The comparison of sequences and determination of percent homology between two sequences can be accomplished using a mathematical algorithm. Preferably, the alignment can be performed using the Clustal Method. Multiple alignment parameters include GAP Penalty=10, Gap Length Penalty=10. For DNA alignments, the pairwise alignment parameters can be Htuple=2, Gap penalty=5, Window=4, and Diagonal saved=4. For protein alignments, the pairwise alignment parameters can be Ktuple=1, Gap penalty=3, Window=5, and Diagonals Saved=5.

In a preferred embodiment, the percent identity between two amino acid sequences is determined using the Needleman and Wunsch (J. Mol. Biol. (48):444-453 (1970)) algorithm which has been incorporated into the GAP program in the GCG software package (available online), using either a Blossom 62 matrix or a PAM250 matrix, and a gap weight of 16, 14, 12, 10, 8, 6, or 4 and a length weight of 1, 2, 3, 6, 4, 5, 7, or 6. In yet another preferred embodiment, the percent identity between two nucleotide sequences is determined using the GAP program in the GCG software package (available online), using a NWSgapdna.CMP matrix and a gap weight of 40, 50, 60, 70, or 80 and a length weight of 1, 2, 3, 6, 4, 5, 7, or 6. In another embodiment, the percent identity between two amino acid or nucleotide sequences is determined using the algorithm of E. Meyers and W. Miller (CABIOS, 4:11-17 (1989)) which has been incorporated into the ALIGN program (version 2.0) (available online), using a PAM120 weight residue table, a gap length penalty of 12 and a gap penalty of 4.

An isolated nucleic acid molecule encoding a TAGLN2 polypeptide homologous to the protein of SEQ ID NOS: 4, 5, 7, or fragment thereof, can be created by introducing one or more nucleotide substitutions, additions or deletions into the nucleotide sequence of SEQ ID NOS: 1, 2, 3, 6, or fragment thereof, or a homologous nucleotide sequence such that one or more amino acid substitutions, additions or deletions are introduced into the encoded protein. Mutations can be introduced into SEQ ID NOS: 1, 2, 3, 6, or fragment thereof, or the homologous nucleotide sequence by standard techniques, such as site-directed mutagenesis and PCR-mediated mutagenesis. Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), bet217-420ranched side chains (e.g., threonine, valine, isoleucine) and aromatic side chains (e.g., tyrosine, phenylalanine, tryptophan, histidine). Thus, a predicted nonessential amino acid residue in TAGLN2 polypeptide is preferably replaced with another amino acid residue from the same side chain family. Alternatively, in another embodiment, mutations can be introduced randomly along all or part of a TAGLN2 polypeptide coding sequence, such as by saturation mutagenesis, and the resultant mutants can be screened for TAGLN2 polypeptide activity described herein to identify mutants that retain TAGLN2 polypeptide activity. Following mutagenesis of SEQ ID NOS: 1, 2, 3, 6, or fragment thereof, the encoded protein can be expressed recombinantly (as described herein) and the activity of the protein can be determined using, for example, assays described herein.

TAGLN2 polypeptide levels may be assessed by any of a wide variety of well-known methods for detecting expression of a transcribed molecule or protein. Non-limiting examples of such methods include immunological methods for detection of proteins, protein purification methods, protein function or activity assays, nucleic acid hybridization methods, nucleic acid reverse transcription methods, and nucleic acid amplification methods.

In preferred embodiments, TAGLN2 polypeptide levels are ascertained by measuring gene transcript (e.g., mRNA), by a measure of the quantity of translated protein, or by a measure of gene product activity. Expression levels can be monitored in a variety of ways, including by detecting mRNA levels, protein levels, or protein activity, any of which can be measured using standard techniques. Detection can involve quantification of the level of gene expression (e.g., genomic DNA, cDNA, mRNA, protein, or enzyme activity), or, alternatively, can be a qualitative assessment of the level of gene expression, in particular in comparison with a control level. The type of level being detected will be clear from the context.

In a particular embodiment, the TAGLN2 polypeptide mRNA expression level can be determined both by in situ and by in vitro formats in a biological sample using methods known in the art. The term “biological sample” is intended to include tissues, cells, biological fluids and isolates thereof, isolated from a subject, as well as tissues, cells and fluids present within a subject. Many expression detection methods use isolated RNA. For in vitro methods, any RNA isolation technique that does not select against the isolation of mRNA can be utilized for the purification of RNA from cells (see, e.g., Ausubel et al., ed., Current Protocols in Molecular Biology, John Wiley & Sons, New York 1987-1999). Additionally, large numbers of tissue samples can readily be processed using techniques well known to those of skill in the art, such as, for example, the single-step RNA isolation process of Chomczynski (1989, U.S. Pat. No. 4,843,155).

The isolated mRNA can be used in hybridization or amplification assays that include, but are not limited to, Southern or Northern analyses, polymerase chain reaction analyses and probe arrays. One preferred diagnostic method for the detection of mRNA levels involves contacting the isolated mRNA with a nucleic acid molecule (probe) that can hybridize to the mRNA encoded by the gene being detected. The nucleic acid probe can be, for example, a full-length cDNA, or a portion thereof, such as an oligonucleotide of at least 7, 15, 30, 50, 100, 250 or 500 nucleotides in length and sufficient to specifically hybridize under stringent conditions to a mRNA or genomic DNA encoding TAGLN2 polypeptide. Other suitable probes for use in the diagnostic assays of the invention are described herein. Hybridization of an mRNA with the probe indicates that TAGLN2 polypeptide is being expressed.

In one format, the mRNA is immobilized on a solid surface and contacted with a probe, for example by running the isolated mRNA on an agarose gel and transferring the mRNA from the gel to a membrane, such as nitrocellulose. In an alternative format, the probe(s) are immobilized on a solid surface and the mRNA is contacted with the probe(s), for example, in a gene chip array, e.g., an Affymetrix™ gene chip array. A skilled artisan can readily adapt known mRNA detection methods for use in detecting the level of the TAGLN2 mRNA expression levels.

An alternative method for determining the TAGLN2 mRNA expression level in a sample involves the process of nucleic acid amplification, e.g., by RT-PCR (the experimental embodiment set forth in Mullis, 1987, U.S. Pat. No. 4,683,202), ligase chain reaction (Barany, 1991, Proc. Natl. Acad. Sci. USA, 88:189-193), self-sustained sequence replication (Guatelli et al., 1990, Proc. Natl. Acad. Sci. USA 87:1874-1878), transcriptional amplification system (Kwoh et al., 1989, Proc. Natl. Acad. Sci. USA 86:1173-1177), Q-Beta Replicase (Lizardi et al., 1988, Bio/Technology 6:1197), rolling circle replication (Lizardi et al., U.S. Pat. No. 5,854,033) or any other nucleic acid amplification method, followed by the detection of the amplified molecules using techniques well-known to those of skill in the art. These detection schemes are especially useful for the detection of nucleic acid molecules if such molecules are present in very low numbers. As used herein, amplification primers are defined as being a pair of nucleic acid molecules that can anneal to 5′ or 3′ regions of a gene (plus and minus strands, respectively, or vice-versa) and contain a short region in between. In general, amplification primers are from about 10 to 30 nucleotides in length and flank a region from about 50 to 200 nucleotides in length. Under appropriate conditions and with appropriate reagents, such primers permit the amplification of a nucleic acid molecule comprising the nucleotide sequence flanked by the primers.

For in situ methods, mRNA does not need to be isolated from the cells prior to detection. In such methods, a cell or tissue sample is prepared/processed using known histological methods. The sample is then immobilized on a support, typically a glass slide, and then contacted with a probe that can hybridize to the TAGLN2 mRNA.

As an alternative to making determinations based on the absolute TAGLN2 polypeptide expression level, determinations may be based on the normalized TAGLN2 polypeptide expression level. Expression levels are normalized by correcting the absolute TAGLN2 polypeptide expression level by comparing its expression to the expression of a non-TAGLN2 polypeptide gene, e.g., a housekeeping gene that is constitutively expressed. Suitable genes for normalization include housekeeping genes such as the actin gene, or epithelial cell-specific genes. This normalization allows the comparison of the expression level in one sample, e.g., a subject sample, to another sample, e.g., a normal sample, or between samples from different sources.

The level or activity of a TAGLN2 can also be detected and/or quantified by detecting or quantifying the expressed polypeptide. The TAGLN2 polypeptide can be detected and quantified by any of a number of means well known to those of skill in the art. These may include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, flow cytometry, Western blotting, and the like. A skilled artisan can readily adapt known protein/antibody detection methods for use in determining whether cells express TAGLN2 polypeptide.

b. Recombinant Expression Vectors and Host Cells

Another aspect of the invention pertains to the use of vectors, preferably expression vectors, containing a nucleic acid encoding a TAGLN2 polypeptide (or a portion thereof). In some embodiments, the vector comprises: (1) a first nucleic acid encoding a chimeric antigen receptor (CAR); and (2) a second nucleic acid encoding TAGLN2. In some embodiments, the vector comprises: (1) a first nucleic acid encoding a chimeric endocrine receptor (CER) comprises: a) the β subunit of the follicle-stimulating hormone (FSHβ), b) a spacer, c) a transmembrane domain, and d) an intracellular domain; and (2) a second nucleic acid encoding TAGLN2. In some embodiments, the vector comprises: (1) a first nucleic acid encoding a chimeric endocrine receptor (CER) comprises: a) the β subunit of the follicle-stimulating hormone (FSHβ), b) a glycine/serine spacer, c) a CD8α transmembrane domain, and d) a 4-1BB-CD3ζ (BBZ) intracellular domain; and (2) a cDNA encoding TAGLN2.

As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. In one embodiment, adenoviral vectors comprising a TAGLN2 nucleic acid molecule are used.

The recombinant expression vectors of the present invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, CA (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein.

The recombinant expression vectors of the invention can be designed for the expression of TAGLN2 in prokaryotic or eukaryotic cells. For example, TAGLN2 can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, CA (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. (1988) Gene 67:31-40), pMAL (New England Biolabs, Beverly, MA) and pRIT5 (Pharmacia, Piscataway, NJ) which fuse glutathione S-transferase (GST), maltose E binding protein, or protein A, respectively, to the target recombinant protein. In one embodiment, the coding sequence of TAGLN2 is cloned into a pGEX expression vector to create a vector encoding a fusion protein comprising, from the N-terminus to the C-terminus, and/or GST-thrombin cleavage site-TAGLN2. The fusion protein can be purified by affinity chromatography using glutathione-agarose resin. Recombinant TAGLN2 polypeptide unfused to GST can be recovered by cleavage of the fusion protein with thrombin.

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, California (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident λ prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, S., Gene Expression Technology: Methods in Enzymology 185, Academic Press, San Diego, California (1990) 119-128). Another strategy is to alter the nucleic acid sequence of the nucleic acid to be inserted into an expression vector so that the individual codons for each amino acid are those preferentially utilized in E. coli (Wada et al. (1992) Nucleic Acids Res. 20:2111-2118). Such alterations of nucleic acid sequences of the invention can be carried out by standard DNA synthesis techniques.

In another embodiment, the TAGLN2 expression vector is a yeast expression vector. Examples of vectors for expression in yeast S. cerivisae include pYepSec1 (Baldari, et al., (1987) EMBO J. 6:229-234), pMFa (Kurjan and Herskowitz, (1982) Cell 30:933-943), pJRY88 (Schultz et al., (1987) Gene 54:113-123), and pYES2 (Invitrogen Corporation, San Diego, CA).

Alternatively, TAGLN2 can be expressed in insect cells using baculovirus expression vectors. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed, B. (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, NY, 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the murine hox promoters (Kessel and Gruss (1990) Science 249:374-379) and the α-fetoprotein promoter (Campes and Tilghman (1989) Genes Dev. 3:537-546).

Another aspect of the present invention pertains to host cells into which a recombinant expression vector or nucleic acid of the present invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, TAGLN2 polypeptide can be expressed in bacterial cells such as E. coli, insect cells, yeast or mammalian cells (such as Fao hepatoma cells, primary hepatocytes, Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual. 2nd, ed., Cold Spring Harbor Laboratory, Cold Spring Harbor Laboratory Press, Cold Spring Harbor, NY, 1989), and other laboratory manuals.

A cell culture includes host cells, media and other byproducts. Suitable media for cell culture are well known in the art. A TAGLN2 polypeptide or fragment thereof, may be secreted and isolated from a mixture of cells and medium containing the polypeptide. Alternatively, a TAGLN2 polypeptide or fragment thereof, may be retained cytoplasmically and the cells harvested, lysed and the protein or protein complex isolated. A TAGLN2 polypeptide or fragment thereof, may be isolated from cell culture medium, host cells, or both using techniques known in the art for purifying proteins, including ion-exchange chromatography, gel filtration chromatography, ultrafiltration, electrophoresis, and immunoaffinity purification with antibodies specific for particular epitopes of TAGLN2 or a fragment thereof.

In some embodiments, TAGLN2 polypeptide, or biologically active fragment thereof, and may be fused to a heterologous polypeptide. In certain embodiments, the fused polypeptide has greater half-life and/or cell permeability than the corresponding unfused TAGLN2 polypeptide, or biologically active fragment thereof. For example, TAGLN2 polypeptide may be fused to a cell permeable peptide to facilitate the delivery of the TAGLN2 polypeptide into the intact cells. Cell Permeable Peptides, also known as Protein Transduction Domains (PTDs), are carriers with small peptide domains that can freely cross cell membranes. Several PTDs have been identified that allow a fused protein to efficiently cross cell membranes in a process known as protein transduction. Studies have demonstrated that a TAT peptide derived from the HIV TAT protein has the ability to transduce peptides or proteins into various cells. PTDs have been utilized in anticancer strategy, for example, a cell permeable Bcl-2 binding peptide, cpm1285, shows activity in slowing human myeloid leukemia growth in mice. Cell-permeable phosphopeptides, such as FGFR730pY, which mimics receptor binding sites for specific SH2 domain-containing proteins are potential tools for cancer research and cell signaling mechanism studies. In other embodiments, heterologous tags can be used for purification purposes (e.g., epitope tags and Fc fusion tags), according to standards methods known in the art.

Thus, a nucleotide sequence encoding all or a selected portion of a TAGLN2 polypeptide may be used to produce a recombinant form of the protein via microbial or eukaryotic cellular processes. Ligating the sequence into a polynucleotide construct, such as an expression vector, and transforming or transfecting into hosts, either eukaryotic (yeast, avian, insect or mammalian) or prokaryotic (bacterial cells), are standard procedures. Similar procedures, or modifications thereof, may be employed to prepare recombinant TAGLN2 polypeptides, or fragments thereof, by microbial means or tissue-culture technology in accord with the subject invention.

In another variation, protein production may be achieved using in vitro translation systems. In vitro translation systems are, generally, a translation system which is a cell-free extract containing at least the minimum elements necessary for translation of an RNA molecule into a protein. An in vitro translation system typically comprises at least ribosomes, tRNAs, initiator methionyl-tRNAMet, proteins or complexes involved in translation, e.g., eIF2, eIF3, the cap-binding (CB) complex, comprising the cap-binding protein (CBP) and eukaryotic initiation factor 4F (eIF4F). A variety of in vitro translation systems are well known in the art and include commercially available kits. Examples of in vitro translation systems include eukaryotic lysates, such as rabbit reticulocyte lysates, rabbit oocyte lysates, human cell lysates, insect cell lysates and wheat germ extracts. Lysates are commercially available from manufacturers such as Promega Corp., Madison, Wis.; Stratagene, La Jolla, Calif.; Amersham, Arlington Heights, Ill.; and GIBCO/BRL, Grand Island, N.Y. In vitro translation systems typically comprise macromolecules, such as enzymes, translation, initiation and elongation factors, chemical reagents, and ribosomes. In addition, an in vitro transcription system may be used. Such systems typically comprise at least an RNA polymerase holoenzyme, ribonucleotides and any necessary transcription initiation, elongation and termination factors. In vitro transcription and translation may be coupled in a one-pot reaction to produce proteins from one or more isolated DNAs.

In certain embodiments, the TAGLN2 polypeptide, or fragment thereof, may be synthesized chemically, ribosomally in a cell free system, or ribosomally within a cell. Chemical synthesis may be carried out using a variety of art recognized methods, including stepwise solid phase synthesis, semi-synthesis through the conformationally-assisted re-ligation of peptide fragments, enzymatic ligation of cloned or synthetic peptide segments, and chemical ligation. Native chemical ligation employs a chemoselective reaction of two unprotected peptide segments to produce a transient thioester-linked intermediate. The transient thioester-linked intermediate then spontaneously undergoes a rearrangement to provide the full-length ligation product having a native peptide bond at the ligation site. Full length ligation products are chemically identical to proteins produced by cell free synthesis. Full length ligation products may be refolded and/or oxidized, as allowed, to form native disulfide-containing protein molecules. (see e.g., U.S. Pat. Nos. 6,184,344 and 6,174,530; and T. W. Muir et al., Cuff. Opin. Biotech. (1993): vol. 4, p 420; M. Miller, et al., Science (1989): vol. 246, p 1149; A. Wlodawer, et al., Science (1989): vol. 245, p 616; L. H. Huang, et al., Biochemistry (1991): vol. 30, p 7402; M. Sclmolzer, et al., Int. J. Pept. Prot. Res. (1992): vol. 40, p 180-193; K. Rajarathnam, et al., Science (1994): vol. 264, p 90; R. E. Offord, “Chemical Approaches to Protein Engineering”, in Protein Design and the Development of New therapeutics and Vaccines, J. B. Hook, G. Poste, Eds., (Plenum Press, New York, 1990) pp. 253-282; C. J. A. Wallace, et al., J. Biol. Chem. (1992): vol. 267, p 3852; L. Abrahmsen, et al., Biochemistry (1991): vol. 30, p 4151; T. K. Chang, et al., Proc. Natl. Acad. Sci. USA (1994) 91: 12544-12548; M. Schnlzer, et al., Science (1992): vol., 3256, p 221; and K. Akaji, et al., Chem. Pharm. Bull. (Tokyo) (1985) 33: 184).

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Preferred selectable markers include those which confer resistance to drugs, such as G418, hygromycin and methotrexate. Nucleic acid encoding a selectable marker can be introduced into a host cell on the same vector as that encoding TAGLN2 or can be introduced on a separate vector. Cells stably transfected with the introduced nucleic acid can be identified by drug selection (e.g., cells that have incorporated the selectable marker gene will survive, while the other cells die).

A host cell of the present invention, such as a prokaryotic or eukaryotic host cell in culture, can be used to produce (i.e., express) TAGLN2 polypeptide. Accordingly, the invention further provides methods for producing TAGLN2 polypeptide using the host cells of the invention. In one embodiment, the method comprises culturing the host cell of invention (into which a recombinant expression vector encoding TAGLN2 has been introduced) in a suitable medium until TAGLN2 polypeptide is produced. In another embodiment, the method further comprises isolating TAGLN2 polypeptide from the medium or the host cell.

c. Isolated TAGLN2 Polypeptides

The present invention also provides soluble, purified and/or isolated forms of TAGLN2 polypeptide, or fragments thereof for use according to methods described herein.

In one aspect, a TAGLN2 polypeptide may comprise a full-length TAGLN2 amino acid sequence or a full-length TAGLN2 amino acid sequence with 1 to about 20 conservative amino acid substitutions. Amino acid sequence of any TAGLN2 polypeptide described herein can also be at least 50, 55, 60, 65, 70, 75, 80, 85, 90, 91, 92, 93, 94, 95, 96, 97, 98, 99, or 99.5% identical to an TAGLN2 polypeptide sequence of interest, described herein, well known in the art, or a fragment thereof. In addition, any TAGLN2 polypeptide, or fragment thereof, described herein can modulate (e.g., reduce) one or more of the following biological activities: cancer cell survival, cancer cell proliferation, and tumor size/volume.

In another aspect, the present invention contemplates a composition comprising an isolated TAGLN2 polypeptide and less than about 25%, or alternatively 15%, or alternatively 5%, contaminating biological macromolecules or polypeptides.

The present invention further provides compositions related to producing, detecting, or characterizing a TAGLN2 polypeptide, or fragment thereof, such as nucleic acids, vectors, host cells, and the like. Such compositions may serve as compounds that modulate a TAGLN2 polypeptide's expression and/or activity, such as antisense nucleic acids.

In certain embodiments, a TAGLN2 polypeptide of the invention may be a fusion protein containing a domain which increases its solubility and bioavailability and/or facilitates its purification, identification, detection, and/or structural characterization. In some embodiments, it may be useful to express a TAGLN2 fusion polypeptides in which the fusion partner enhances fusion protein stability in blood plasma and/or enhances systemic bioavailability. Exemplary domains, include, for example, glutathione S-transferase (GST), protein A, protein G, calmodulin-binding peptide, thioredoxin, maltose binding protein, HA, myc, poly arginine, poly His, poly His-Asp or FLAG fusion proteins and tags. Additional exemplary domains include domains that alter protein localization in vivo, such as signal peptides, type 21 secretion system-targeting peptides, transcytosis domains, nuclear localization signals, etc. In various embodiments, a TAGLN2 polypeptide of the invention may comprise one or more heterologous fusions. Polypeptides may contain multiple copies of the same fusion domain or may contain fusions to two or more different domains. The fusions may occur at the N-terminus of the polypeptide, at the C-terminus of the polypeptide, or at both the N- and C-terminus of the polypeptide. It is also within the scope of the invention to include linker sequences between a polypeptide of the invention and the fusion domain in order to facilitate construction of the fusion protein or to optimize protein expression or structural constraints of the fusion protein. In one embodiment, the linker is a linker described herein, e.g., a linker of at least 1, 2, 3, 6, 4, 5, 7, 6, 7, 8, 9, 10, 15, 20 or more amino acids. The linker can be, e.g., an unstructured recombinant polymer (URP), e.g., a URP that is 9, 10, 11, 12, 13, 14, 15, 20 amino acids in length, i.e., the linker has limited or lacks secondary structure, e.g., Chou-Fasman algorithm. An exemplary linker comprises (e.g., consists of) the amino acid sequence GGGGAGGGG (SEQ ID NO: 9). In another embodiment, the polypeptide may be constructed so as to contain protease cleavage sites between the fusion polypeptide and polypeptide of the invention in order to remove the tag after protein expression or thereafter. Examples of suitable endoproteases, include, for example, Factor Xa and TEV proteases.

In some embodiments, TAGLN2 polypeptides, or fragments thereof, are fused to an antibody (e.g., IgG1, IgG2, IgG3, IgG4) fragment (e.g., Fc polypeptides). Techniques for preparing these fusion proteins are known, and are described, for example, in WO 99/31241 and in Cosman et. al. (2001) Immunity 14:123-133. Fusion to an Fc polypeptide offers the additional advantage of facilitating purification by affinity chromatography over Protein A or Protein G columns.

In still another embodiment, a TAGLN2 polypeptide may be labeled with a fluorescent label to facilitate their detection, purification, or structural characterization. In an exemplary embodiment, a TAGLN2 polypeptide of the invention may be fused to a heterologous polypeptide sequence which produces a detectable fluorescent signal, including, for example, green fluorescent protein (GFP), enhanced green fluorescent protein (EGFP), Renilla Reniformis green fluorescent protein, GFPmut2, GFPuv4, enhanced yellow fluorescent protein (EYFP), enhanced cyan fluorescent protein (ECFP), enhanced blue fluorescent protein (EBFP), citrine and red fluorescent protein from discosoma (dsRED).

An “isolated” or “purified” protein or biologically active portion thereof is substantially free of cellular material when produced by recombinant DNA techniques, or chemical precursors or other chemicals when chemically synthesized. The language “substantially free of cellular material” includes preparations of TAGLN2 polypeptide in which the polypeptide is separated from cellular components of the cells in which it is naturally or recombinantly produced. In one embodiment, the language “substantially free of cellular material” includes preparations of TAGLN2 polypeptide having less than about 30% (by dry weight) of non-TAGLN2 polypeptide (also referred to herein as a “contaminating protein”), more preferably less than about 20% of non-TAGLN2 polypeptide, still more preferably less than about 10% of non-TAGLN2 polypeptide, and most preferably less than about 5% non-TAGLN2 polypeptide. When the TAGLN2 polypeptide or biologically active portion thereof is recombinantly produced, it is also preferably substantially free of culture medium, i.e., culture medium represents less than about 20%, more preferably less than about 10%, and most preferably less than about 5% of the volume of the protein preparation. The language “substantially free of chemical precursors or other chemicals” includes preparations of TAGLN2 polypeptide in which the polypeptide is separated from chemical precursors or other chemicals which are involved in the synthesis of the polypeptide. In one embodiment, the language “substantially free of chemical precursors or other chemicals” includes preparations of TAGLN2 polypeptide having less than about 30% (by dry weight) of chemical precursors of non-TAGLN2 polypeptide chemicals, more preferably less than about 20% chemical precursors of non-TAGLN2 polypeptide chemicals, still more preferably less than about 10% chemical precursors of non-TAGLN2 polypeptide chemicals, and most preferably less than about 5% chemical precursors of non-TAGLN2 polypeptide chemicals. In preferred embodiments, isolated polypeptides or biologically active portions thereof lack contaminating polypeptides from the same animal from which the TAGLN2 polypeptide is derived. Typically, such polypeptides are produced by recombinant expression of, for example, a human TAGLN2 polypeptide in a nonhuman cell.

In preferred embodiments, the protein or portion thereof comprises an amino acid sequence which is sufficiently homologous to an amino acid sequence of SEQ ID NOS: 4, 5, 7, or fragment thereof, such that the protein or portion thereof maintains one or more of the following biological activities or, in complex, modulates (e.g., reduce) one or more of the following biological activities: cancer cell survival, cancer cell proliferation, and tumor size/volume. The portion of the protein is preferably a biologically active portion as described herein. In another preferred embodiment, the TAGLN2 polypeptides has an amino acid sequence shown in SEQ ID NOS: 4, 5, 7, or fragment thereof, respectively, or an amino acid sequence which is at least about 50%, 55%, 60%, 65%, 70%, 75%, 80%, 85%, 90%, 91%, 92%, 93%, 94%, 95%, 96%, 97%, 98%, 99% or more homologous to the amino acid sequence shown in SEQ ID NOS: 4, 5, 7, or fragment thereof. In yet another preferred embodiment, the TAGLN2 polypeptide has an amino acid sequence which is encoded by a nucleotide sequence which hybridizes, e.g., hybridizes under stringent conditions, to the nucleotide sequence of SEQ ID NOS: 1, 2, 3, 6, or fragment thereof, or a nucleotide sequence which is at least about 50%, preferably at least about 60%, more preferably at least about 70%, yet more preferably at least about 80%, still more preferably at least about 90%, and most preferably at least about 95% or more homologous to the nucleotide sequence shown in SEQ ID NOS: 1, 2, 3, 6, or fragment thereof. The preferred TAGLN2 polypeptides of the present invention also preferably possess at least one of the TAGLN2 polypeptide biological activities described herein.

Biologically active portions of a TAGLN2 protein include peptides comprising amino acid sequences derived from the amino acid sequence of the TAGLN2 protein, e.g., the amino acid sequence shown in SEQ ID NOS: 4, 5, 7, or fragment thereof, or the amino acid sequence of a protein homologous to the TAGLN2 protein, which include fewer amino acids than the full-length TAGLN2 protein or the full-length polypeptide which is homologous to the TAGLN2 protein, and exhibit at least one activity of the TAGLN2 protein. Typically, biologically active portions (peptides, e.g., peptides which are, for example, 50, 55, 60, 65, 70, 75, 80, 85, 90, 95, 100 or more amino acids in length) comprise a domain or motif, (e.g., the full-length protein minus the signal peptide). In a preferred embodiment, the biologically active portion of the protein which includes one or more the domains/motifs described herein can modulate (e.g., reduce) one or more the following biological activities: cancer cell survival, cancer cell proliferation, and tumor size/volume. Moreover, other biologically active portions, in which other regions of the protein are deleted, can be prepared by recombinant techniques and evaluated for one or more of the activities described herein. Preferably, the biologically active portions of the TAGLN2 protein include one or more selected domains/motifs or portions thereof having biological activity. In one embodiment, a TAGLN2 fragment of interest consists of a portion of a full-length TAGLN2 protein that is less than 240, 230, 220, 210, 200, 195, 190, 185, 180, 175, 170, 165, 160, 155, 150, 145, 140, 135, 130, 125, 120, 115, 110, 105, 100, 95, 90, 85, 80, 75, or 70 amino acids in length.

TAGLN2 proteins can be produced by recombinant DNA techniques. For example, a nucleic acid molecule encoding the protein is cloned into an expression vector (as described above), the expression vector is introduced into a host cell (as described above) and the TAGLN2 protein is expressed in the host cell. The TAGLN2 protein can then be isolated from the cells by an appropriate purification scheme using standard protein purification techniques. Alternative to recombinant expression, a TAGLN2 protein, polypeptide, or peptide can be synthesized chemically using standard peptide synthesis techniques. Moreover, native TAGLN2 protein can be isolated from cells, for example using an anti-TAGLN2 antibody.

The invention also provides TAGLN2 chimeric or fusion proteins. As used herein, a TAGLN2 “chimeric protein” or “fusion protein” comprises a TAGLN2 polypeptide operatively linked to a non-TAGLN2 polypeptide. An “TAGLN2 polypeptide” refers to a polypeptide having an amino acid sequence corresponding to TAGLN2, whereas a “non-TAGLN2 polypeptide” refers to a polypeptide having an amino acid sequence corresponding to a protein which is not substantially homologous to the TAGLN2 protein, e.g., a protein which is different from the TAGLN2 protein and which is derived from the same or a different organism. Within the fusion protein, the term “operatively linked” is intended to indicate that the TAGLN2 polypeptide and the non-TAGLN2 polypeptide are fused in-frame to each other. The non-TAGLN2 polypeptide can be fused to the N-terminus or C-terminus of the TAGLN2 polypeptide. For example, in one embodiment the fusion protein is a TAGLN2-GST and/or TAGLN2-Fc fusion protein in which the TAGLN2 sequences, respectively, are fused to the N-terminus of the GST or Fc sequences. Such fusion proteins can be made using TAGLN2 polypeptides. Such fusion proteins can also facilitate the purification, expression, and/or bioavailability of recombinant TAGLN2. In another embodiment, the fusion protein is a TAGLN2 protein containing a heterologous signal sequence at its C-terminus. In certain host cells (e.g., mammalian host cells), expression and/or secretion of TAGLN2 can be increased through use of a heterologous signal sequence.

Preferably, a TAGLN2 chimeric or fusion protein of the invention is produced by standard recombinant DNA techniques. For example, DNA fragments coding for the different polypeptide sequences are ligated together in-frame in accordance with conventional techniques, for example by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, for example, Current Protocols in Molecular Biology, eds. Ausubel et al. John Wiley & Sons: 1992). Moreover, many expression vectors are commercially available that already encode a fusion moiety (e.g., a GST polypeptide). A TAGLN2-encoding nucleic acid can be cloned into such an expression vector such that the fusion moiety is linked in-frame to the TAGLN2 protein.

The present invention also pertains to homologues of the TAGLN2 proteins. Homologues of the TAGLN2 protein can be generated by mutagenesis, e.g., discrete point mutation or truncation of the TAGLN2 protein, respectively. As used herein, the term “homologue” refers to a variant form of the TAGLN2 protein. In one embodiment, treatment of a subject with a homologue having a subset of the biological activities of the naturally occurring form of the protein has fewer side effects in a subject relative to treatment with the naturally occurring form of the TAGLN2 protein.

In an alternative embodiment, homologues of the TAGLN2 protein can be identified by screening combinatorial libraries of mutants, e.g., truncation mutants, of the TAGLN2 protein. In one embodiment, a variegated library of TAGLN2 variants is generated by combinatorial mutagenesis at the nucleic acid level and is encoded by a variegated gene library. A variegated library of TAGLN2 variants can be produced by, for example, enzymatically ligating a mixture of synthetic oligonucleotides into gene sequences such that a degenerate set of potential TAGLN2 sequences is expressible as individual polypeptides, or alternatively, as a set of larger fusion proteins (e.g., for phage display) containing the set of TAGLN2 sequences therein. There are a variety of methods which can be used to produce libraries of potential TAGLN2 homologues from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential TAGLN2 sequences. Methods for synthesizing degenerate oligonucleotides are known in the art (see, e.g., Narang, S. A. (1983) Tetrahedron 39:3; Itakura et al. (1984) Annu. Rev. Biochem. 53:323; Itakura et al. (1984) Science 198:1056; Ike et al. (1983) Nucleic Acid Res. 11:477.

In addition, libraries of fragments of the TAGLN2 protein coding can be used to generate a variegated population of TAGLN2 fragments for screening and subsequent selection of homologues of a TAGLN2 protein. In one embodiment, a library of coding sequence fragments can be generated by treating a double stranded PCR fragment of an TAGLN2 coding sequence with a nuclease under conditions wherein nicking occurs only about once per molecule, denaturing the double stranded DNA, renaturing the DNA to form double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal, C-terminal and internal fragments of various sizes of the TAGLN2 protein.

Several techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of TAGLN2 homologues. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique which enhances the frequency of functional mutants in the libraries, can be used in combination with the screening assays to identify TAGLN2 homologues (Arkin and Youvan (1992) Proc. Natl. Acad. Sci. USA 89:7811-7815).

### Cell Therapy

In another aspect encompassed by the present invention, the methods include adoptive cell therapy, whereby genetically engineered cells (e.g., T cells) co-expressing a binding protein (e.g., a CAR such as a CER) and TAGLN2 are administered to subjects. Such administration can increase cell surface level of FABP5 in the T cells and/or fatty acid uptake of the T cells, thus enhances one or more of mitochondrial respiration, microtubule dynamics, organization of cytoskeleton, proliferation, activation, and/or cytotoxicity of the T cells.

In some embodiments, provided herein are an engineered T cell expressing a chimeric antigen receptor (CAR) and comprising a transgene encoding TAGLN2. In some embodiments, the CAR is a chimeric endocrine receptor (CER). In some embodiments, the chimeric endocrine receptor (CER) comprises: a) the β subunit of the follicle-stimulating hormone (FSHβ), b) a spacer, c) a transmembrane domain, and d) an intracellular domain. In some embodiments, the FSHβ comprises a FSHβ signal peptide at the 5′ end. In some embodiments, the spacer is a glycine/serine spacer. In some embodiments, the transmembrane domain is a CD8α transmembrane domain. In some embodiments, the intracellular domain is a 4-1BB-CD3ζ (BBZ) intracellular domain (ICD). In some embodiments, the T cell expresses TAGLN2. In some embodiments, the T cell has a higher surface level of FABP5, a higher lipid uptake, and/or a higher cytotoxicity compared to a T cell without the transgene encoding TAGLN2. In some embodiments, the T cell is a cytotoxic T lymphocyte (CTL). In some embodiments, the T cell is a primary CD8⁺ T cell.

In some aspects, provided herein are methods of generating a CAR-expressing T cell comprising contacting the immune cell with (1) a first nucleic acid encoding a CAR; and (2) a second nucleic acid encoding TAGLN2. In some embodiments, the CAR is a chimeric endocrine receptor (CER). In some embodiments, the chimeric endocrine receptor (CER) comprises: a) the β subunit of the follicle-stimulating hormone (FSHβ), b) a spacer, c) a transmembrane domain, and d) an intracellular domain. In some embodiments, the FSHβ comprises a FSHβ signal peptide at the 5′ end. In some embodiments, the spacer is a glycine/serine spacer. In some embodiments, the transmembrane domain is a CD8α transmembrane domain. In some embodiments, the intracellular domain is a 4-1BB-CD3ζ (BBZ) intracellular domain (ICD).

In some aspects, the provided methods and uses include methods and uses for adoptive cell therapy. In some embodiments, the methods include administration of the cells or a composition containing the cells to a subject, tissue, or cell, such as one having, at risk for, or suspected of having the disease, condition or disorder. In some embodiments, the cells, populations, and compositions are administered to a subject having the particular disease or condition to be treated (e.g., via adoptive cell therapy, such as by adoptive T cell therapy). In some embodiments, the cells or compositions are administered to the subject, such as a subject having or at risk for the disease or condition. In some embodiments, the methods thereby treat, e.g., ameliorate one or more symptom of the disease or condition.

Methods for administration of cells for adoptive cell therapy are known and may be used in connection with the provided methods and compositions (e.g., U.S. Pat. Publ. No. 2003/0170238, U.S. Pat. No. 4,690,915, Rosenberg (2011) Nat. Rev. Clin. Oncol. 8:577-585, Themeli et al. (2013) Nat. Biotechnol. 31:928-933, Tsukahara et al. (2013) Biochem. Biophys. Res. Commun. 438:84-89, and Davila et al. (2013) PLoS ONE 8:e61338).

In some embodiments, cell therapy (e.g., adoptive cell therapy, such as adoptive T cell therapy) may be carried out by autologous transfer, in which the cells are isolated and/or otherwise prepared from the subject who is to receive the cell therapy, or from a sample derived from such a subject. Thus, in some embodiments, the cells are derived from a subject, e.g., a patient, in need of a treatment and the cells, following isolation and processing are administered to the same subject.

In some embodiments, the cell therapy (e.g., adoptive cell therapy, such as adoptive T cell therapy) may be carried out by allogeneic transfer, in which the cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical (syngeneic). In some embodiments, the first and second subjects are genetically similar. In some embodiments, the second subject expresses the same HLA class or supertype as the first subject.

In some embodiments, the subject, to whom the cells, cell populations, or compositions are administered is a primate, such as a human. In some embodiments, the primate is a monkey or an ape. The subject may be male or female and may be any suitable age, including infant, juvenile, adolescent, adult, and geriatric subjects. In some embodiments, the subject is a non-primate mammal, such as a rodent. In some examples, the patient or subject is a validated animal model for disease, adoptive cell therapy, and/or for assessing toxic outcomes such as cytokine release syndrome (CRS).

The cells (e.g., T cells) co-expressing a binding protein (e.g., a CAR such as a CER) and TAGLN2 may be administered by any suitable means, for example, by injection, e.g., intravenous or subcutaneous injections, intraocular injection, periocular injection, subretinal injection, intravitreal injection, trans-septal injection, subscleral injection, intrachoroidal injection, intracameral injection, subconjectval injection, subconjuntival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. Dosing and administration may depend in part on whether the administration is brief or chronic. Various dosing schedules include but are not limited to single or multiple administrations over various time-points, bolus administration, and pulse infusion.

For the prevention or treatment of disease, the appropriate dosage of the cells may depend on the type of disease to be treated, the type of binding molecule, the severity and course of the disease, whether the binding molecule is administered for preventive or therapeutic purposes, previous therapy, the patient's clinical history and response to the binding molecule, and the discretion of the attending physician. The compositions and molecules and cells are in some embodiments suitably administered to the patient at one time or over a series of treatments.

In some embodiments, the cells, or individual populations of sub-types of cells, may be administered to the subject at a range of about one million to about 100 billion cells and/or that amount of cells per kilogram of body weight, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges and/or per kilogram of body weight. Dosages may vary depending on attributes particular to the disease or disorder and/or patient and/or other treatments.

In some embodiments, for example, where the subject is a human, the dose includes fewer than about 1×10⁸ total binding protein (e.g., CAR)-expressing cells, T cells, or peripheral blood mononuclear cells (PBMCs), e.g., in the range of about 1×10⁶ to 1×10⁸ such cells, such as 2×10⁶, 5×10⁶, 1×10⁷, 5×10⁷, or 1×10⁸ or total such cells, or the range between any two of the foregoing values.

In some embodiments, the cells or related compositions described herein, such as nucleic acids, host cells, pharmaceutical formulations, and the like, may be administered as part of a combination treatment, such as simultaneously with or sequentially with, in any order, another therapeutic intervention, such as another antibody or engineered cell or receptor or agent, such as a cytotoxic or therapeutic agent.

In some embodiments, the cells or related composition may be co-administered with one or more additional therapeutic agents or in connection with another therapeutic intervention, either simultaneously or sequentially in any order. In some contexts, the cells or related composition are co-administered with another therapy sufficiently close in time such that the cell populations enhance the effect of one or more additional therapeutic agents, or vice versa. In some embodiments, the cells or related composition are administered prior to the one or more additional therapeutic agents. In some embodiments, the cells or related composition are administered after to the one or more additional therapeutic agents.

In some embodiments, the biological activity of the cells or related composition is measured by any of a number of known methods once the cells or related composition are administered to a mammal (e.g., a human). Parameters to assess include specific binding of an engineered or natural T cell or other immune cell to antigen, in vivo, e.g., by imaging, or ex vivo, e.g., by ELISA or flow cytometry. In some embodiments, the ability of the cells to destroy target cells (cytotoxicity) may be measured using any suitable assay or method known in the art (e.g., Kochenderfer et al. (2009) J. Immunother. 32: 689-702 and Herman et al. (2004) J. Immunol. Meth. 285:25-40). In some embodiments, the biological activity of the cells also may be measured by assaying expression and/or secretion of certain cytokines, such as CD107a, IFNγ, IL-2, and TNF alpha. In some embodiments, the biological activity is measured by assessing clinical outcomes, such as reduction in tumor volume.

In some embodiments, cells are modified in any number of ways, such that their therapeutic or prophylactic efficacy is increased. For example, the binding protein (e.g., engineered CAR, or antigen-binding fragment thereof) expressed by the population may be conjugated either directly or indirectly through a linker to a targeting moiety. The practice of conjugating compounds to targeting moieties is well-known in the art (e.g., Wadwa et al. (1995) J. Drug Targeting 3:111 and U.S. Pat. No. 5,087,616).

Immune cells, such as cytotoxic lymphocytes, may be obtained from any suitable source such as peripheral blood, spleen, and lymph nodes. The immune cells may be used as crude preparations or as partially purified or substantially purified preparations, which may be obtained by standard techniques, including, but not limited to, methods involving immunomagnetic or flow cytometry techniques using antibodies.

In another aspect encompassed by the present invention, a method for treatment of cancer, the method comprising administering to the subject an effective amount of the cells described herein expressing a binding protein (e.g., engineered CAR, or antigen-binding fragment thereof) and TAGLN2. In one embodiment, the cells are administered systemically, such as by injection. Alternately, one may administer locally rather than systemically, for example, via injection directly into tissue, such as in a depot or sustained release formulation. As described above, single or multiple administrations of the cells described herein expressing a binding protein (e.g., engineered CAR, or antigen-binding fragment thereof) cells, either alone or in combination with the lymphocytes, may be carried out with cell numbers and treatment being selected by the care provider (e.g., physician). Similarly, the cells, either alone or in combination with lymphocytes, may be administered in a pharmaceutically acceptable carrier. Suitable carriers may be growth medium in which the cells were grown, or any suitable buffering medium such as phosphate buffered saline. Cells may be administered alone or as an adjunct therapy in conjunction with other therapeutics.

### Pharmaceutical Compositions and Methods

The compositions described herein may be used in a variety of diagnostic, prognostic, and therapeutic applications. In any method described herein, such as a diagnostic method, prognostic method, therapeutic method, or combination thereof, all steps of the method can be performed by a single actor or, alternatively, by more than one actor. For example, diagnosis can be performed directly by the actor providing therapeutic treatment. Alternatively, a person providing a therapeutic agent can request that a diagnostic assay be performed. The diagnostician and/or the therapeutic interventionist can interpret the diagnostic assay results to determine a therapeutic strategy. Similarly, such alternative processes can apply to other assays, such as prognostic assays.

In an aspect encompassed by the present invention, provided herein are methods for treating cancer (e.g., metastatic ovarian cancer (OvCa) such as high-grade serous OvCa (HGSOC). In some embodiments, the method comprises administering to a subject a therapeutically effective amount of an agent that increases expression, level, and/or activity of Transgelin-2 (TAGLN2) in T cells. In some embodiments, the method comprises administering to a subject a therapeutically effective amount of a composition comprising cells (e.g., T cells) expressing a chimeric antigen receptor (CAR) and comprising a transgene encoding TAGLN2 described herein.

The methods described herein may be used to treat a subject in need thereof. As used herein, a “subject in need thereof” includes any subject who has cancer (e.g., metastatic ovarian cancer (OvCa) such as high-grade serous OvCa (HGSOC)).

The pharmaceutical compositions disclosed herein may be delivered by any suitable route of administration, including parenterally. In some embodiments the pharmaceutical compositions are delivered generally (e.g., via parenteral administration). In specific embodiments, the pharmaceutical compositions are administered by infusion.

Actual dosage levels of the active ingredients in the pharmaceutical compositions may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular agent employed, the route of administration, the time of administration, the rate of excretion or metabolism of the particular agent being employed, the duration of the treatment, other drugs, compounds and/or materials used in combination with the particular agent employed, the age, sex, weight, condition, general health and prior medical history of the patient being treated, and like factors well-known in the medical arts.

A physician or veterinarian having ordinary skill in the art can readily determine and prescribe the effective amount of the pharmaceutical composition required. For example, the physician or veterinarian could prescribe and/or administer doses of the agents employed in the pharmaceutical composition at levels lower than that required in order to achieve the desired therapeutic effect and gradually increase the dosage until the desired effect is achieved.

In general, a suitable daily dose of an agent described herein will be that amount of the agent which is the lowest dose effective to produce a therapeutic effect. Such an effective dose will generally depend upon the factors described above.

A pharmaceutical dosage unit may be an effective amount or part of an effective amount. An “effective amount” is to be understood herein as an amount or dose of active ingredients required to prevent and/or reduce the symptoms of a disease (e.g., cancer (e.g., metastatic ovarian cancer (OvCa)) relative to an untreated patient. The effective amount of active compound(s) used in accordance with the present invention for preventive and/or therapeutic treatment of cancer (e.g., metastatic ovarian cancer (OvCa) such as high-grade serous OvCa (HGSOC)) varies depending upon the manner of administration, the age, body weight, and general health of the subject. Ultimately, the attending physician or veterinarian will decide the appropriate amount and dosage regimen. Such amount is referred to as an “effective” amount. This effective amount may also be the amount that is able to induce an effective cellular T cell response in the subject to be treated, or more such as an effective systemic cellular T cell response.

In another aspect encompassed by the present invention, the methods provided herein include administering to both human and non-human mammals. Veterinary applications also are contemplated. In some embodiments, the subject may be any living organism in which an immune response may be elicited. Examples of subjects include, without limitation, humans, livestock, dogs, cats, mice, rats, and transgenic species thereof.

In some embodiments, the pharmaceutical composition may be administered at any time that is appropriate. For example, the administering may be conducted before or during treatment of a subject having a cancer (e.g., metastatic ovarian cancer (OvCa) such as high-grade serous OvCa (HGSOC)), and continued after the cancer cells become clinically undetectable. The administering also may be continued in a subject showing signs of recurrence.

In some embodiments, the pharmaceutical composition may be administered in a therapeutically or a prophylactically effective amount. Administering the pharmaceutical composition to the subject may be carried out using known procedures, and at dosages and for periods of time sufficient to achieve a desired effect.

In some embodiments, the pharmaceutical composition may be administered to the subject at any suitable site. The route of administering may be parenteral, intramuscular, subcutaneous, intradermal, intraperitoneal, intranasal, intravenous (including via an indwelling catheter), via an afferent lymph vessel, or by any other route suitable in view of the subject's condition. In some embodiments, the dose may be administered in an amount and for a period of time effective in bringing about a desired response, be it eliciting the immune response or the prophylactic or therapeutic treatment of cancer (e.g., metastatic ovarian cancer (OvCa) such as high-grade serous OvCa (HGSOC)) and symptoms associated therewith.

The pharmaceutical composition may be given subsequent to, preceding, or contemporaneously with other therapies including therapies that also elicit an immune response in the subject For example, the subject may previously or concurrently be treated by other forms of immunomodulatory agents, such other therapies may be provided in such a way so as not to interfere with the immunogenicity of the compositions described herein.

Administering may be properly timed by the care giver (e.g., physician, veterinarian), and may depend on the clinical condition of the subject, the objectives of administering, and/or other therapies also being contemplated or administered. In some embodiments, an initial dose may be administered, and the subject monitored for an immunological and/or clinical response. An immunological reaction also may be determined by a delayed inflammatory response at the site of administering. One or more doses subsequent to the initial dose may be given as appropriate, typically on a monthly, semimonthly, or a weekly basis, until the desired effect is achieved. Thereafter, additional booster or maintenance doses may be given as required, particularly when the immunological or clinical benefit appears to subside.

In general, an appropriate dosage and treatment regimen provides the active molecules or cells in an amount sufficient to provide a benefit. Such a response may be monitored by establishing an improved clinical outcome (e.g., more frequent remissions, complete or partial, or longer disease-free survival) in treated subjects as compared to non-treated subjects. Increases in preexisting immune responses to a tumor generally correlate with an improved clinical outcome. Such immune responses may generally be evaluated using standard proliferation, cytotoxicity or cytokine assays, which are routine.

As used herein, administration of a composition refers to delivering the same to a subject, regardless of the route or mode of delivery. Administration may be effected continuously or intermittently, and parenterally. Administration may be for treating a subject already confirmed as having a recognized condition, disease or disease state, or for treating a subject susceptible to or at risk of developing such a condition, disease or disease state. Co-administration with an adjunctive therapy may include simultaneous and/or sequential delivery of multiple agents in any order and on any dosing schedule (e.g., engineered immune cells with one or more cytokines; immunosuppressive therapy such as calcineurin inhibitors, corticosteroids, microtubule inhibitors, low dose of a mycophenolic acid prodrug, or any combination thereof).

In some embodiments, a plurality of doses of a host cell (e.g., an engineered immune cell) described herein is administered to the subject, which may be administered at intervals between administrations of about two to about four weeks.

Treatment or prevention methods encompassed by the present invention may be administered to a subject as part of a treatment course or regimen, which may comprise additional treatments prior to, or after, administration of the instantly disclosed unit doses, cells, or compositions. For example, in some embodiments, a subject receiving a unit dose of the host cell (e.g., an engineered immune cell) is receiving or had previously received a hematopoietic cell transplant (HCT; including myeloablative and non-myeloablative HCT). In any of the foregoing embodiments, a hematopoietic cell used in an HCT may be a “universal donor” cell that is modified to reduce or eliminate expression of one or more endogenous genes that encode a polypeptide product selected from an MHC, antigen, and a binding protein (e.g., by a chromosomal gene knockout according to the methods described herein).

Techniques and regimens for performing cell transplantation are known in the art and may comprise transplantation of any suitable donor cell, such as a cell derived from umbilical cord blood, bone marrow, or peripheral blood, a hematopoietic stem cell, a mobilized stem cell, or a cell from amniotic fluid. Accordingly, in some embodiments, a host cell (e.g., an engineered immune cell) encompassed by the present invention may be administered with or shortly after stem cell therapy.

Methods encompassed by the present invention may, in some embodiments, further include administering one or more additional agents to treat the disease or disorder (e.g., cancer (e.g., metastatic ovarian cancer (OvCa)) in a combination therapy. For example, in some embodiments, a combination therapy comprises administering engineered CAR-T cells expressing TAGLN2, or TAGLN2 nucleic acids or polypeptides encompassed by the present invention with (concurrently, simultaneously, or sequentially) an anti-cancer agent. In some embodiments, a combination therapy comprises administering engineered CAR-T cells expressing TAGLN2, or TAGLN2 nucleic acids or polypeptides encompassed by the present invention with one or more of an immunotherapy (e.g., a checkpoint inhibitor), a chemotherapy, a targeted therapy, a hormone therapy, and a surgery. In some embodiments, a combination therapy comprises administering an engineer T cell, composition, or unit dose of the engineer T cells encompassed by the present invention with a secondary therapy, such as a surgery, an antibody, a hormone, or any combination thereof.

In some embodiments, the subject is a human, such as a human with cancer (e.g., metastatic ovarian cancer (OvCa) such as high-grade serous OvCa (HGSOC)). In some embodiments, the subject is a rodent, such as a mouse.

The disclosure is further illustrated by the following examples which should not be construed as limiting.

### EXAMPLES

### Example 1: Materials and Methods

### Human Specimens

Plasma samples from cancer-free women were obtained from the New York Blood Center. Malignant ascites fluid samples, peripheral blood and solid tumors were obtained from patients with Stage III-IV HGSOC were procured through Surgical Pathology at Weill Cornell Medicine, Memorial Sloan-Kettering Cancer Center and Moffit Cancer Center. All specimens were acquired with informed consent, classified as surgical discard, and kept de-identified for subsequent experimental analyses. The ascites fluid underwent initial processing by centrifugation at 4° C. for 10 minutes at 1,300 rpm, with subsequent separation of supernatants from cell pellets and filtration through 0.22-am filters to eliminate cellular debris. Processed samples were cryopreserved at −80° C. in small aliquots until use. Red blood cells in cell pellets were lysed with ACK lysing buffer (Gibco). Tumor infiltrating CD8 T cells (CD45⁺CD20⁻CD14⁻CD3⁺CD8⁺) were sorted from malignant ascites using a BD FACS Aria 11 SORP cell sorter at Flow Cytometry Core Facility in Weill Cornell Medicine. Dead cells were excluded using the DAPI. All OvCa specimens used and analyzed in this study are described in Tables 1A-1C.

TABLE 1A
Information on collected human HGSOC patient-derived specimens.

| Sample | Patient | Purpose of | |
|---|---|---|---|
| type | ID | experiment | Patient information |
| Ascites | A28 | In vitro analyses | Primary OvCa or peritoneal cancer; Chemo-naive |
| | A29 | (Cell-free | Primary OvCa; Chemo-naive |
| | A30 | supernatants) | Metastatic OvCa; Primary surgical debulking; |
| | | | Chemo-naive |
| | A35 | | Primary OvCa; Chemo-naive |
| | A43 | | Primary OvCa; Chemo-naive |
| | A20 | TIL analyses | High-grade serous OvCa; Chemo-naive |
| | A21 | | Undetermined |
| | A22 | | Recurrent OvCa Stage 1C |
| | A25 | | Recurrent OvCa |
| | A26 | | Recurrent peritoneal OvCa; Multi-drug resistant; |
| | | | Several times of Chemotherapies |
| | A28 | | Primary OvCa or peritoneal cancer; Chemo-naive |
| | A30 | | High-grade serous OvCa; Chemo-naive |
| | A33 | | High-grade serous endometrial or ovarian carcinoma |
| | A35 | | High-grade serous OvCa; Chemo-naive |
| | A36 | | High-grade serous OvCa; Chemo-naive |
| | A38 | | High-grade serous OvCa; Chemo-naive |
| | A39 | | Mucinous OvCa, Stage lll; Chemo-naive |
| | A40 | | Adenocarcinoma; cytology high grade; Mullerian |
| | | | origin (includes ovarian and endometrium) |
| | A41 | | Undetermined |
| | A46 | | Recurrent Fallopian tube cancer; |
| | | | Neoadjuvant chemotherapy |
| | A3 | Sort | High-grade serous OvCa |
| | A6 | CD45⁺CD3⁺CD8⁺ | High-grade serous OvCa |
| | A7 | TILs for qRT-PCR | High-grade serous OvCa |
| | A9 | analyses | High-grade serous OvCa |
| | A14 | | High-grade serous OvCa; platinum-resistant, |
| | | | Recurrent peritoneal OvCa |
| | A15 | | High-grade serous OvCa |
| | A16 | | High-grade serous OvCa; terminal stage |
| | A17 | | High-grade serous OvCa; Chemo-naive |
| | A18 | | High-grade serous OvCa; Chemo-naive |
| | A19 | | High-grade serous OvCa; Chemo-naive |
| | A28 | | Primary OvCa or peritoneal cancer; Chemo-naive |
| | A29 | | Primary OvCa; Chemo-naive |
| | A30 | | High-grade serous OvCa; Chemo-naive |
| | A35 | | High-grade serous OvCa; Chemo-naive |
| | A36 | | High-grade serous OvCa; Chemo-naive |
| | A38 | | High-grade serous OvCa; Chemo-naive |

TABLE 1B

| Sample | | Purpose of | |
|---|---|---|---|
| type | Patient ID | experiment | Patient information |
| PBMC | 06-107-8-A | CD8⁺ T cell | High-grade serous OvCa; Chemo-naive |
| | 06-107-9A | analysis for lipid | High-grade serous OvCa; Chemo-naive |
| | 06-107-14A | uptake, FABP5 | High-grade serous OvCa; Chemo-naive |
| | 06-107-28A | surface/total | High-grade serous OvCa; Chemo-naive |
| | 06-107-33A | expression levels/ | High-grade serous OvCa; Chemo-naive |
| | 06-107-47A | Tagln2 | High-grade serous OvCa; Chemo-naive |
| | 06-107-49A | expression levels | High-grade serous OvCa; Chemo-naive |
| | 06-107-53A | | High-grade serous OvCa; Chemo-naive |

TABLE 1C

| Patient | | Purpose of | |
|---|---|---|---|
| ID | Sample type | experiment | Patient information |
| 1491 | Left ovary-tumor | CD8⁺ T cell analysis | Mucinous adenocarcinoma of |
| | PBMC | for lipid uptake, | ovary; grade 1 |
| 14159 | Left ovary-tumor | FABP5 surface/total | Carcinosarcoma |
| | PBMC | expression levels/ | |
| 1581 | Right ovary-tumor | Tagln2 expression | Poorly differentiated ovarian |
| | PBMC | levels | carcinoma with mixed clear |
| | | | cell/papillary serous carcinoma |
| | | | and lesser endometrioid |
| | | | carcinoma pattern |
| 15105 | Omentum- tumor | | High-grade carcinoma, mixed |
| | PBMC | | serous and clear cell types |

### Transgenic Mice and Experimental OvCa Models

C57BL_/6J, B6.SJL-Ptprc^{a} Pepc^{b}BoyJ (CD45.1), Eif2ak3^{fl/fl}, Atf6^{fl/fl} and Vav1^{Cre} mice were obtained from The Jackson Laboratory. Fabp5 KO mice were provided by M. Kaczocha⁵². Tagln2^{fl/fl} mice where exon 3 of the Tagln2 gene was flanked by two loxp sites were newly generated by the Mouse Genetics Core Facility at Memorial Sloan Kettering Cancer Center. Ern1^{fl/fl} and Xbp1^{fl/fl} mice have been previously described by our group^{17,53,54}. We generated conditional-KO mice lacking PERK or ATF6 in leukocytes by crossing Eij2ak3^{fl/fl} or Atf6^{fl/fl} mice, respectively, with the Vav1^{Cre} strain that allows selective gene deletion in hematopoietic cells,^{55,56}. To generate conditional KO mice lacking TAGLN2, IRE1α or XBP1 specifically in T cells, Tagln2^{fl/fl}, Ern1^{fl/fl} or Xbp1^{fl/fl} mice were crossed with the Cd4^{Cre} strain, respectively. Female mice were housed in pathogen-free microisolator cages at the animal facilities of Memorial Sloan Kettering Cancer Center and Weill Cornell Medicine and used at 8 to 12 weeks of age for all experiments. Functional and survival experiments were conducted using age-matched, littermate controls. In vivo experiments included three to sixteen mice per group, based on transgenic genotype and sex availability. Mice were handled in compliance with the Institutional Animal Care and Use Committee procedures and guidelines under protocol 2011-0098.

The aggressive ID8-Defb29/Vegfa derivate was cultured and used as previously described^{22,57}. The PPNM cell line (Trp53^{-/-R172H}Pten^{-/-}Nf1^{-/-}MycOE) was generously provided by Dr. R. Weinberg under MTA⁴⁶. The MP cell line was generated as described⁵¹. For tumor implantation, 1.5×10⁶ ID8-based ovarian cancer cells suspended in 200 μl of sterile PBS were intraperitoneally (i.p.) injected into mice. PPNM cells were suspended in PBS containing Matrigel (Corning Matrigel matrix, Cat #47743-716) at 1:1 ratio, and 200 μl of the mix containing 5×10⁵ cells were administered i.p. into mice, as reported. Alternatively, 2.0×10⁶ MP cells were suspended in 200 μl of sterile PBS was intraperitoneally (i.p.) injected into mice. Metastatic progression, ascites accumulation, and host survival were monitored over time. Tumor burden in the peritoneal cavity was assessed by live bioluminescent imaging. Briefly, PPNM-bearing mice were given a single i.p. injection of VivoGlo luciferin (2 mg/mouse. Promega, Cat #P1042) and then imaged on a Xenogen IVIS Spectrum In Vivo imaging system at the Weill Cornell Research Animal Resource Center. All cell lines were verified for mycoplasma contamination and maintained under prophylactic plasmocin supplementation (Invivogen, Cat #ant-mpt).

### RNA Isolation and Real Time Quantitative PCR (RT-qPCR) Analysis

Total RNA was isolated using the QIAzol lysis reagent (Qiagen) according to the manufacturer's instructions. RNA (0.1-1 μg) was reverse-transcribed to generate cDNA using the qScript cDNA synthesis kit (Quantabio). Quantitative RT-PCR was performed using PerfeCTa SYBR green fastmix (Quantabio) on a QuantStudio 6 Flex real-time PCR system (Applied Biosystems). Normalized gene expression was calculated by the comparative, threshold cycle method using ACTB for human or Actb for mouse as endogenous controls. All primers used in this study are described in Table 3.

TABLE 3
Primer Sequences.

| | | | | |
|---|---|---|---|---|
| | | SEQ | | SEQ |
| | | ID | | ID |
| Name | Forward Primer (5′-3′) | NO: | Reverse Primer (5′-3′) | NO: |

Primers for RT-qPCR (Human)

| | | | | |
|---|---|---|---|---|
| ACTB | GCGAGAAGATGACCCAGATC | 10 | CCAGTGGTACGGCCAGAGG | 29 |
| TAGLN2 | ATGGCACGGTGCTATGTGAG | 11 | CCCACCCAGATTCATCAGCG | 30 |
| XBP1s | CTGAGTCCGCAGCAGGTG | 12 | TCCAAGTTGTCCAGAATGCC | 31 |
| FABP5 | TGAAGGAGCTAGGAGTGGGAA | 13 | TGCACCATCTGTAAAGTTGCAG | 32 |
| CD36 | AAGCCAGGTATTGCAGTTCTTT | 14 | GCATTTGCTGATGTCTAGCACA | 33 |
| FABP4 | ACTGGGCCAGGAATTTGACG | 15 | CTCGTGGAAGTGACGCCTT | 34 |
| IFNG | TCGGTAACTGACTTGAATGTCCA | 16 | TCGCTTCCCTGTTTTAGCTGC | 35 |
| TNFA | GGAGAAGGGTGACCGACTCA | 17 | CTGCCCAGACTCGGCAA | 36 |
| GZMB | CCCTGGGAAAACACTCACACA | 18 | GCACAACTCAATGGTACTGTCG | 37 |

Primers for RT-qPCR (Mouse)

| | | | | |
|---|---|---|---|---|
| Actb | CTCAGGAGGAGCAATGATCTTGAT | 19 | TACCACCATGTACCCAGGCA | 38 |
| Tagln2_Exon3 | CTTGAGGCTCACCACAGGAA | 20 | TTGAAGGCCATCGAAGAGGC | 39 |
| Tagln2 | TCTTTGCCATCACCACAGCT | 21 | CGTGCCGTCCTTGAGCCACT | 40 |
| | GCTCAGAATG | | TCTGGAAGTT | |
| Xbp1s | AAGAACACGCTTGGGAATGG | 22 | CTGCACCTGCTGCGGAC | 41 |
| Sec61al | CTATTTCCAGGGCTTCCGAGT | 23 | AGGTGTTGTACTGGCCTCGGT | 42 |
| Dnajb9/Erdj4 | TAAAAGCCCTGATGCTGAAGC | 24 | TCCGACTATTGGCATCCGA | 43 |

Primers for luciferase reporter construct generation

| | | | | |
|---|---|---|---|---|
| Tagln2- | GGGGTACCCCCACCCCTCAA | 25 | CCGCTCGAGCGGCGTCCAAGA | 44 |
| promoter | CTATTGCT | | GGGCTGG | |

Primers for ChIP-qPCR

| | | | | |
|---|---|---|---|---|
| XBP1s non- | AGGGGTAGAAAAGTGCCTGC | 26 | CAGTGAGGTCACTCCTTGCC | 45 |
| binding site | | | | |
| (NB) | | | | |
| XBP1s | AGTTAAATGGCAAGCAGAACCAC | 27 | GCGCCTTCCTACAGGATAGAGTA | 46 |
| binding site 1 | | | | |
| (BS1) | | | | |
| XBP1s | TTCCTGCCTACTGACCACCT | 28 | GCAAGGGCCAAGAGGGTTTA | 47 |
| binding site 2 | | | | |
| (BS2) | | | | |

T Cell siRNA and mRNA Electroporation

T cells were transfected with either small interfering RNA (siRNA) or messenger RNA (mRNA) via electroporation utilizing the Neon Transfection System (Thermo Fisher Scientific). The following siRNAs and mRNAs were used: siRNA-ON-TARGETplus Non-targeting Control Pool (Horizon), ON-TARGETplus Mouse Fabp5 siRNA (Horizon). mRNA-CleanCap® Ovalbumin mRNA (5-methoxyuridine; L-7210) was obtained from TriLink Biotechnology. Mouse Tagln2 and human FABP5 mRNAs were designed and synthesized by TriLink Biotechnology and Genscript, respectively. Briefly, naïve splenic CD8⁺ T cells were isolated and stimulated with plate-bound anti-CD3F (145-2C11, 5 μg/ml) and soluble anti-CD28 (37.51, 1 μg/ml; BD Pharmingen) antibodies for 24 hours. On the following day, activated viable T cells were counted and resuspended in Neon Buffer T, then mixed with indicated siRNA or mRNA. The mixture was pipetted using the Neon pipette and tips and plugged the Neon pipette into position in the Neon transfection device. Electroporation was performed at 3 pulses, 10 ms pulse width, 1600V. T cells were subsequently expanded on plate-bound anti-CD3F and soluble anti-CD28 antibodies in complete medium with 100 units per mL of IL-2 (200-02; PeproTech) for additional 2 days.

### Flow Cytometry

Flow cytometry was conducted using fluorochrome-conjugated antibodies purchased from BioLegend, unless stated otherwise. Cells were washed with PBS, Fc-gamma receptor-blocked using TruStain fcX™ (anti-mouse CD16/32, 93), LIVE/DEAD™ Fixable Near-IR dead cell stain for live/dead discrimination (Invitrogen) and then stained for surface or intracellular markers at 4° C. in the dark for 30 minutes. For staining of mouse cells we used: anti-CD45 (30-F11, 1:200), anti-CD3 (17A2, 1:200), anti-CD4 (RM4-5, 1:200), anti-CD8α (53-6.7, 1:200), anti-CD44 (IM7, 1:200), anti-CD62L (MEL-14, 1:200) anti-CD11c (N418, 1:200), anti-I-A/I-E (M5/114.15.2, 1:200; Tonbo biosciences), anti-CD11b (M1/70, 1:200), anti-F4/80 (BM8, 1:200), anti-NK1.1 (PK136, 1:200), anti-CD19 (6D5, 1:200), CD45.1 (A20, 1:200), CD45.2 (104, 1:200; BD Pharmingen), anti-FABP5 (primary; AF1476, 1:100; R&D Systems, secondary; Donkey anti-Goat IgG (H+L) Alexa Fluor™ Plus 647; A32849, 1:200; Invitrogen), anti-Transgelin-2 (primary; 15508-1-AP, 1:100; Thermo Fisher Scientific, secondary; Alexa Fluor™ 488 goat anti-rabbit IgG (H+L); A11008, 1:200; Invitrogen; R-phycoerythrin goat anti-rabbit IgG (H+L); P2771MP, 1:200; Invitrogen), anti-TOX (TXRX10, 1:50; Thermo Fisher Scientific), anti-Ki-67 (16A8, 1:50), anti-IFNγ (XMG1.2, 1:100), anti-TNFα (MP6-XT22, 1:100), anti-GranzymeB (QA16A02, 1:100). To stain human cells we used: anti-CD45 (HI30, 1:200), anti-CD3 (HIT3a, 1:200), anti-CD8α (HIT8a, 1:200), anti-CD19 (HIB19, 1:200), anti-CD45RO (UCHL1, 1:200), 5 anti-CCR7 (150503, 1:200; BD Pharmingen), anti-CD44 (515, 1:200; BD Pharmingen), anti-XBP1s (Q3-695, 1:50; BD Pharmingen), anti-FABP5 (primary; AF1476, 1:100; R&D Systems, secondary; Donkey anti-Goat IgG (H+L) Alexa Fluor™ Plus 647; A32849, 1:200; Invitrogen), anti-FABP4 (primary; BAF1476, 1:100; R&D Systems, secondary; Donkey anti-Goat IgG (H+L) Alexa FluorT^{m} Plus 647; A32849, 1:200; Invitrogen), anti-CD36 (Y23-1185, 1:50), anti-Transgelin-2 (primary; 15508-1-AP, 1:100; Thermo Fisher Scientific, secondary; Alexa Fluor™ 488 goat anti-rabbit IgG (H+L); A11008, 1:200; Invitrogen). Staining of transcription factor XBP1s, intracellular proteins (Ki-67, TAGLN2, Total FABP5, FABP4, CD36 and TOX) and cytokines (IFNγ, TNFα and GranzymeB) was carried out using Foxp3/transcription factor staining buffer set (eBioscience) according to the manufacturer's instructions. For in vitro lipid uptake experiments, 2×10⁵ human or mouse CD8⁺ T cells in complete medium containing 1 μmol/L BODIPY™ 500/510 C1, C12 (D3823; Thermo Fisher Scientific) for 30 minutes at 37° C. with 5% CO2. To measure the mitochondrial mass and membrane potential, WT or Tagln2 KO CD8⁺ T cells pre-activated for twenty-four hours in complete medium were cultured in complete medium or glucose-free medium with or without oleic acids for forty-eight hours, respectively. Then, cells were stained with 10 nM MitoTracker Deep Red (M46753; Thermo Fisher Scientific) and 100 nM MitoTracker Green (M46750; Thermo Fisher Scientific) for 15 min at 37° C. with 5% CO2 followed by Live/Dead and cell surface staining. Flow cytometry was performed on a LSRII or a Fortessa-X20 instruments (BD Biosciences) and data were analyzed using FlowJo v.10 (TreeStar).

### Immunoprecipitation and LC-MS/MS Analysis

For LC-MS/MS analysis of FABP5 interacting proteins in CD8⁺ T cells, activated mouse CD8⁺ T cell pellets were lysed using Pierce™ IP Lysis Buffer (Thermo Fisher Scientific, Cat #87787) supplemented with a protease and phosphatase inhibitor tablet (Millipore, Cat #11697498001 and Roche, Cat #04906837001) for 30 min at 4° C. Homogenates were centrifuged at 15,000 rpm. for 10 min at 4° C., and the supernatants were collected. Protein concentrations were determined using a BCA protein assay kit (Thermo Fisher Scientific, Cat #23225). Lysates were then incubated for 3 hours with Dynabeads™ MyOne™ Streptavidin T1 (65601; Invitrogen) under continuous rotation to remove non-specific proteins. After bead removal, goat IgG biotinylated control (BAF108; R&D systems) or goat anti-mouse FABP5 biotinylated antibodies (BAF1476; R&D Systems) was added for overnight incubation at 4° C. under continuous rotation, and then incubation with Dynabeads™ MyOne™ Streptavidin T1 was carried out for 2 hours at 4° C. under continuous rotation to capture immune complexes. After wash by PBS containing 0.05% tween-20, immune complexes were eluted from the magnetic beads with 0.1M glycine-HCl (pH2.02) with mixing for 10 min, followed by neutralized with 1M Tris (pH 8.5). IgG or FABP5 pull-down elution sample was fractionated on 4-12% Bis-Tris gels (Invitrogen) and running gels were stained with SimplyBlue™ SafeStain Coomassie (LC6060; Invitrogen) mass spectrometry analyses, which were performed by the Proteomics and Lipidomics Core Facility of Weill Cornell Medicine following standard methods.

### Co-Immunoprecipitation Assays

To validate the interaction between FABP5 and TAGLN2, activated mouse CD8⁺ T cell pellets were lysed using Pierce™ IP Lysis Buffer (Thermo Fisher Scientific, Cat #87787) supplemented with a protease and phosphatase inhibitor tablet (Millipore, Cat #11697498001 and Roche, Cat #04906837001) for 30 min at 4° C. Homogenates were centrifuged at 15,000 rpm. for 30 min at 4° C., and the supernatants were collected. Protein concentrations were determined using a BCA protein assay kit (Thermo Fisher Scientific, Cat #23225). Lysates were precleared by adding Protein A/G magnetic beads (88803; Thermo Fisher Scientific) for 1 hour at 4° C. under continuous rotation. After bead removal, goat IgG isotype control (02-6202; Invitrogen) or goat anti-mouse FABP5 (AF1476; R&D Systems) antibodies for FABP5 immunoprecipitation experiment or rabbit IgG isotype control (026102; Invitrogen) or rabbit anti-mouse Transgelin-2 (15508-1-AP; Thermo Fisher Scientific) for TAGLN2 immunoprecipitation experiment was added for overnight incubation at 4° C. under continuous rotation, and then incubation with Protein A/G magnetic beads (88803; Thermo Fisher Scientific) was carried out for 1 hours at room temperature under continuous rotation to capture immune complexes. After washing by 1× TBS containing 0.05% tween-20 and 0.5M NaCl, beads were removed by magnetic. Each precipitated sample was separated via SDS-PAGE and transferred onto PVDF membranes following standard protocols. The following antibodies were used for western blotting: anti-FABP5 (AF1476) (R&D Systems), anti-Transgelin-2 (15508-1-AP) (Thermo Fisher Scientific), Rabbit anti-Goat IgG Fc secondary antibody conjugated with HRP (Thermo Fisher Scientific, Cat #31433), Goat anti-Rabbit IgG Fe secondary antibody conjugated with HRP (Thermo Fisher Scientific, Cat #A16116). SuperSignal West Pico (Thermo Fisher Scientific, Cat #34580) or Femto chemiluminescent substrates (Thermo Fisher Scientific, Cat #34095) were used to image blots in an iBright CL 1000 instrument (Thermo Fisher Scientific).

### Lipidomics

Fatty acid species in malignant ascites, or CD8⁺ T cell samples were analyzed and quantified using LC-MS at the Lipidomics Core Facility of Wayne State University School of Medicine.

### Cytokine Quantification

Human undiluted ascites samples were submitted to Eve Technologies™ Assay Services for analysis using the Human Cytokine/Chemokine 71-Plex Discovery Assay® Array.

### Plasmid Constructs and Luciferase Reporter Assays

Expression constructs used for luciferase-based assays are XBP-1 2: pFLAG.XBP1p.CMV2 (Addgene plasmid #21833) and Flag-p50(1-435) (Addgene plasmid #44747) while reporter construct used is pGL3-Tagln2 promoter (−738 to +134). For dual luciferase reporter assays, 2×10⁴ HEK-293T cells were plated overnight in flat bottom 96-well plates and transfected with the indicated plasmids using jetPRIME (Polyplus-transfection) according to the manufacturer's protocol. Briefly, 18 ng of reporter and 2 ng of Renilla plasmid were co-transfected with various ratios (wt:wt) of expression plasmids (reporter: expression plasmid=1:1, 1:2 or 1:3) and pcDNA3.1, which was added to reach a total 200 ng of plasmid per well. After forty-eight hours, cells were washed with PBS and lysed Passive Lysis Buffer according to the manufacturer's protocol (Dual Luciferase Reporter Assay System, #E1960; Promega). Firefly and Renilla luciferase activities were measured in white-bottom 96-well plates using an automated luminometer (SpectraMax iD3 Multi-Mode Microplate Reader; Molecular Devices). The reporter activity (Firefly) was normalized to its own Renilla luciferase activity.

### Chromatin Immunoprecipitation and PCR (ChIP-PCR)

Pre-activated mouse CD8⁺ T cells were incubated in complete medium in the presence or absence of tunicamycin (1 μg/ml) for 16 hours. Cells were then washed and fixed in 1% formaldehyde for ChIP assay. Cross-linking was terminated using 0.125M glycine. Nuclear extracts were collected and resuspended in a lysis buffer containing a high salt concentration. Chromatin sonication was carried out using a cell disruptor (Branson 150D Sonifier 150). The chromatin solution was precleared by adding Protein A/G magnetic beads (88803; Thermo Fisher Scientific) for 1 hour at 4° C. under continuous rotation. After bead removal, anti-mouse IgG1 (MOPC-21; Biolegend) or anti-mouse XBP1s (9D11A43; Biolegend) antibodies were added for overnight incubation at 4° C. under continuous rotation, and then incubation with Protein A/G magnetic beads was carried out for 2 hours at 4° C. under continuous rotation. Beads were removed by magnetic and sequentially washed with lysis buffer high salt, wash buffer and elution buffer. Cross-links were reversed by heating at 65° C. in a water bath, and the DNA bound to the beads isolated by extraction with phenol/chloroform/isoamylalcohol. Within the regions of interest, XBP1s binding is represented as the percentage of enrichment over input, and calculation was conducted using the change-in-threshold (2^{-ΔΔCT}) method. The sequences for primer pairs used for ChIP-quantitative PCR analyses are mentioned in Table 3.

### Immunofluorescence and Confocal Microscopy

Naïve or activated CD8⁺ T cells in the absence or presence of tunicamycin (2.5-3×10⁶) were transferred onto poly-L-lysine pre-coated glass coverslips (Neuvitro) and incubated at 37° C. with 5% CO2 for 30 min. The coverslips were washed with cold PBS three times between each step. Cells were subsequently fixed with ice-cold acetone for 5 min at room temperature, warmed 2% formaldehyde for 20 min at room temperature and ice-cold 70% ethanol for 5 min at 4° C. Then, cells were permeabilized for 20 min in PBS containing 0.5% Triton X-100 and 10% FBS at room temperature. Then, the coverslips were blocked for 1 h in PBS containing 10% BSA at room temperature, followed by incubation at 4° C. overnight with primary antibodies rabbit anti-Transgelin-2 (15508-1-AP, Thermo Fisher Scientific, 1:100) or goat anti-FABP5 (AF1476, R&D Systems, 1:100) in PBS containing 0.1% tween 20 and 1% BSA, protected from light. Then, secondary antibodies Alexa Fluor 488-conjugated goat anti-rabbit IgG (Molecular Probes, 1:1000) for anti-Transgelin-2 or Alexa Fluor™ Plus 647-conjugated donkey anti-goat IgG (Invitrogen, 1:1000) for anti-FABP5 were added for 1 h at room temperature in the dark. Cells were counterstained with 4′,6-diamidino-2-phenylindole (DAPI, Thermo Fisher Scientific, 0.5 μg/ml) for 5 min at room temperature in the dark. After washing and removing excess solution, the flipped coverslips were placed on the mounting medium (Southern Biotechnology). Slides were allowed to dry in the dark for 1 h in a humid chamber at room temperature. Slides were then sealed with fingernail polish before examination. Digital confocal images were captured on a Zeiss LSM 880 Confocal Microscope with the Airy Scan high-resolution detector at the Weill Cornell CLC Imaging Core Facility.

### Colocalization Coefficients

Percentage of colocalization of indicated markers (Manders' coefficient) was calculated using the JACoP plugin on ImageJ software⁵⁸. Thresholds were fixed for each experiment. For quantification, at least 30 cells were analyzed to generate individual coefficients.

### Single-Cell RNA-Sequencing

Mouse: The total CD3⁺ T cells (DAPI-CD45⁺CD11b-CD19-NK1.1-MHC11-CD3+) of peritoneal lavage were isolated from mice bearing ID8-Defb29/Vegfa ovarian cancer for 23 days and subjected to single-cell RNA sequencing (scRNA-seq). Library preparation, sequencing, and raw data preprocessing were performed at the Genomics Resources Core Facility of Weill Cornell Medicine. Raw gene expression matrices were generated for each sample by the Cell Ranger (v.3.0.2) Pipeline coupled with mouse reference version GRCm38 (mm10). The output-filtered gene expression matrices were analyzed by R software (v.4.2.2) with the Seurat package⁵⁹ (v.3.0.0). In brief, genes expressed at a proportion of >0.1% of the data and cells with >200 genes detected were selected for further analyses. Low-quality cells were removed if they met the following criteria: <800 unique molecular identifiers (UMI), <500 genes, or >5% UMIs derived from the mitochondrial genome. Further, gene expression matrices were normalized by the Normalize-Data function, and 2,000 features with high cell-to-cell variation were calculated using the FindVariableFeatures( ) function. To reduce the dimensionality of the data sets, the RunPCA( ) function was conducted with default parameters on linear transformation-scaled data generated by the ScaleData( ) function. In the end, we clustered cells using the FindNeighbors( ) and FindClusters( ) functions and performed nonlinear dimensional reduction with the RunUMAP( ) function with default settings. All details regarding the Seurat analyses performed in this work can be found in the website tutorial (https:satijalab.org/seurat/v3.0/pbmc3k tutorial.html). Data were deposited under NCBI Gene Expression Omnibus (GEO) Accession number GSE248595.

Human: For the analysis of scRNA-seq data of tumor-infiltrating lymphocytes (TILs) in HGSOC patients, preprocessed counts from 11 HGSOC treatment-naïve tumor specimens⁴⁷ were downloaded from Gene Expression Omnibus (GEO) with accession code GSE165897. Deeper annotation of T cell subtypes was obtained by projecting the CD4 and CD8⁺ T cells to a reference atlas using ProjecTILs⁶⁰. Then, differential expression analysis was performed within each subtype comparing cells with and without TALGN2 expression using FindMarkers function from Seurat package⁵⁹ (v4.3.0). From this comparison, the fold-change (in logarithmic scale) was used in a GSEA analysis to test the enrichment of ER stress gene signature (Table 2) in Tagln2^{hi} CD8 T_{EM} TILs using clusterProfiler package⁶¹ (v4.5.0).

TABLE 2
Genes in the ER stress signature used for GSEA analysis.

| GENE | |
|---|---|
| SYMBOL | GENE NAME |
| Hyou1 | hypoxia up-regulated 1 |
| Hspa14 | heat shock protein 14 |
| Sec61a1 | SEC61 translocon subunit alpha 1 |
| Sec24d | SEC24 homolog D, COPII coat complex component |
| Hspa13 | heat shock protein 70 family, member 13 |
| Sec24c | SEC24 homolog C, COPII coat complex component |
| Ambra1 | autophagy/beclin 1 regulator 1 |
| Surf4 | surfeit gene 4 |
| Atg13 | autophagy related 13 |
| Xbp1 | X-box binding protein 1 |
| P4hb | prolyl 4-hydroxylase, beta polypeptide |
| Fam129a | family with sequence similarity 129 member A |
| Pdia4 | protein disulfide isomerase associated 4 |
| Spcs3 | signal peptidase complex subunit 3 homolog (S. cerevisiae) |
| Surf6 | surfeit gene 6 |
| Atf6 | activating transcription factor 6 |
| Dapk1 | death associated protein kinase 1 |
| Dnajb9 | DnaJ heat shock protein family (Hsp40) member B9 |
| Dnajc3 | DnaJ heat shock protein family (Hsp40) member C3 |
| Mfn2 | mitofusin 2 |
| Pdia6 | protein disulfide isomerase associated 6 |
| Ccdc47 | coiled-coil domain containing 47 |
| Dnajc14 | DnaJ heat shock protein family (Hsp40) member C14 |
| Sil1 | SIL1 nucleotide exchange factor |
| Sec16a | SEC16 homolog A, endoplasmic reticulum export factor |
| Tmx3 | thioredoxin-related transmembrane protein 3 |
| Sec23b | SEC23 homolog B, COPII coat complex component |
| SEC31A | SEC31 homolog A, COPII coat complex component |
| Gosr2 | golgi SNAP receptor complex member 2 |
| Asns | asparagine synthetase |
| Atf4 | activating transcription factor 4 |
| Ddit3 | DNA-damage inducible transcript 3 |
| Ddit4 | DNA-damage inducible transcript 4 |
| Edem1 | ER degradation enhancer, mannosidase alpha-like 1 |
| Hspa5 | heat shock protein 5 |

### Seahorse Assays

Purified naïve CD8⁺ T cells from wild type mice were activated with CD3F and CD28 (Purified NA/LE Hamster anti-mouse CD3F (145-2C11) and anti-mouse CD28 (37.51), BD pharmingen). Pre-activated CD8⁺ T cells were then stimulated with or without Tunicamycin at various concentrations for 16 hours at 37° C. with 5% CO2. In some experiments, pre-activated CD8⁺ T cells were overexpressed with mRNAs encoding control ovalbumin (Ctrl-mRNA) or mouse TAGLN2 (Tagln2-mRNA). After treatment, cells were washed and resuspended in nonbuffered RPMI 1640, pH 7.4 (Agilent) supplemented with 10 mM glucose, 2 mM L-glutamine, and 1 mM pyruvate. 2.5×10⁵ cells per well were plated onto poly-D-lysine precoated XF96 cell culture microplates (Agilent Technologies). The oxygen consumption rate (OCR) and extracellular acidification rate (ECAR) were measured on an XFe96 extracellular flux analyzer (Agilent Technologies). After basal OCR and ECAR measurements were obtained, an OCR trace was recorded in response to oligomycin (1 μM), carbonyl cyanide-p-(trifluoromethoxy) phenylhydrazone (FCCP, 1 μM), and rotenone and antimycin (0.5 M each) following the XF Cell Mito Stress test kit (Agilent).

To evaluate the contribution of fatty acids as mitochondrial fuel usage, pre-activated CD8⁺ T cells stimulated with Tunicamycin either overexpressed with mRNAs encoding control ovalbumin (Ctrl-mRNA) or mouse TAGLN2 (Tagln2-mRNA) were seeded in the Seahorse medium containing 10 mM glucose, 2 mM L-glutamine, and 1 mM pyruvate. After recording basal OCR and ECAR measurements, cells were injected with corresponding base medium (control), or etomoxir (4 M, Tocris Bioscience) followed by oligomycin, FCCP, and rotenone and antimycin injection using the XF standard substrate oxidation test (Agilent). Metabolic parameters were calculated as follows: basal respiration=last rate measurement before oligomycin injection−minimum rate measurement after rotenone and antimycin injection; maximal respiration=maximum rate measurement after FCCP injection−minimum rate measurement after rotenone and antimycin injection. At least five technical replicates per sample were examined. After analysis, the cell numbers of each well were determined by nuclear DNA staining with Hoechst 33342 (Sigma), and OCR and ECAR values were normalized accordingly.

### Western Blotting

PPNM cancer cells were washed twice in 1× cold PBS and cell pellets were lysed using RIPA lysis and extraction buffer (Thermo Fisher Scientific, Cat #89900) supplemented with a protease and phosphatase inhibitor tablet (Millipore, Cat #11697498001 and Roche, Cat #04906837001). Homogenates were centrifuged at 14,000 rpm. for 30 min at 4° C., and the supernatants were collected. Protein concentrations were determined using a BCA protein assay kit (Thermo Fisher Scientific, Cat #23225). Equivalent amounts of protein were separated by SDS-PAGE and transferred onto PVDF membranes following the standard protocol. The following antibodies were used: anti-FSHR (PA5-95380; Thermo Fisher Scientific), HRP-Conjugated Beta Actin Monoclonal Antibody (Thermo Fisher Scientific, Cat #MA5-15739-HRP), anti-GAPDH Rabbit Monoclonal Antibody (Cell Signaling Technology, Cat #14C10), Goat anti-Rabbit secondary antibody conjugated with I-IRP (Thermo Fisher Scientific, Cat #32460). SuperSignal West Pico (Thermo Fisher Scientific, Cat #34580) was used to image blots in an iBright CL1000 instrument (Thermo Fisher Scientific).

### Plasmids and Retroviral Transduction

pBMN-I-GFP and pBMN-I-GFP-FSHCER retroviral vectors were kindly provided by J. R. Conejo-Garcia⁵⁰. Tagln2 cDNA was subcloned with T2A sequences in pBMN-I-GFP-FSHCER (pBMN-I-GFP-FSHCER-2A-Tagln2) vector by Genscript Biotech. Retroviruses were produced by transfecting 6 μg of the retroviral expression vector together with 4 μg of the retroviral packaging vector (pCL-Eco, Addgene plasmid #12371) into retroviral packaging cell line Platinum-E (RV-101, Cell Biolabs, Inc.), using Lipofectamine™ 3000 Transfection Reagent (Thermo Fisher Scientific). Forty-eight hours after transfection, high-titer viral supernatant was collected. For transduction, 2×10⁶ cells/mL of naïve CD8⁺ T cells or Pan T cells from B6.SJL-Ptprc^{a} Pepc^{b}/BoyJ (CD45.1) mice were isolated and activated with plate-bound anti-CD3 and soluble anti-CD28 overnight, followed by spin-infection with viral supernatant for 90 minutes at 2500 rpm, 30° C. in the presence of 8 μg/mL of polybrene. Every 2 days, replenish fresh complete media with added 100 units per mL of IL-2 (200-02; PeproTech) to expand appropriate number of cells for further functional experiments. At day 7, transduced T cells were used for in vitro killing assays or adoptive cell transfer in PPNM ovarian tumor-bearing mice.

### In Vitro Killing Assay

PPNM OvCa cells were plated at a density of 1×10⁴ cells per well in flat bottom 96-well plates containing 200 μL of culture medium. These plates were then incubated overnight at 37° C. with 5% CO2. On the following day, tumor conditioned medium was washed away and added fresh complete medium with no beta-mercaptoethanol and the same number of Mock- or CER-transduced CD8+GFP+ T cells per well (in 200 μL). Following eighteen hours, CD8⁺ T cells and PPNM cells were collected by trypsinization and proceeded to flow cytometric analysis of cellular cytotoxicity (target cells—CD45-CD3-gated PPNM ovarian cancer cells) by using Annexin V Apoptosis Detection Kit with PI (Biolegend).

### Tumor Implantation and CER T Cell Treatments

Wild type C57BL/6J (CD45.2) female mice were implanted via intraperitoneal (i.p.) injection with 5×10⁵ PPNM OvCa cells suspended in PBS containing Matrigel (Corning Matrigel matrix, Cat #47743-716) at 1:1 ratio (200 μL per mouse). Alternatively, 2.0×10⁶ MP cells were suspended in 200 μl of sterile PBS was intraperitoneally (i.p.) injected into mice. After 7 days, tumor-bearing mice were randomized into treatment groups (PPNM model: n=16 each, MP model: n=8 each). On days 8 and 15, mice were treated i.p. with 1×10⁷ cells per mouse, either CER or CER-Tagln2 T cells, which were generated from B6.SJL-Ptprc^{a} Pepcb/BoyJ (CD45.1) mice. For the in vivo assessment of checkpoint blockade using anti-PD-1 antibodies, mice were i.p. treated every 4 days a total of 4 times with isotype control (Bio X Cell; cat. #BE0089) or anti-PD-1 blocking antibodies (Bio X Cell; cat. #BE0146) at 200 g/mouse, starting 9 days after ovarian cancer challenge. Metastatic progression, ascites accumulation, and host survival were monitored over time. Tumor burden in the peritoneal cavity was assessed by live bioluminescent imaging.

### Statistical Analyses

All statistical analyses were performed using GraphPad Prism 10 software. Significance for pairwise correlation analysis was calculated using the Spearman's correlation coefficient (r). Comparisons between two groups were assessed using unpaired or paired (for matched comparisons) two-tailed Student's t-test, or non-parametric Mann-Whitney U-test. Multiple comparisons were assessed by one-way ANOVA, including Tukey's multiple comparisons tests. Survival rates were compared using the log-rank test. Data are presented as mean±s.e.m. Exact p-values are shown, and p-values of <0.05 were considered to be statistically significant.

### Example 2: OvCa Impairs Lipid Uptake and FABP5 Surface Localization in CD8⁺ T Cells

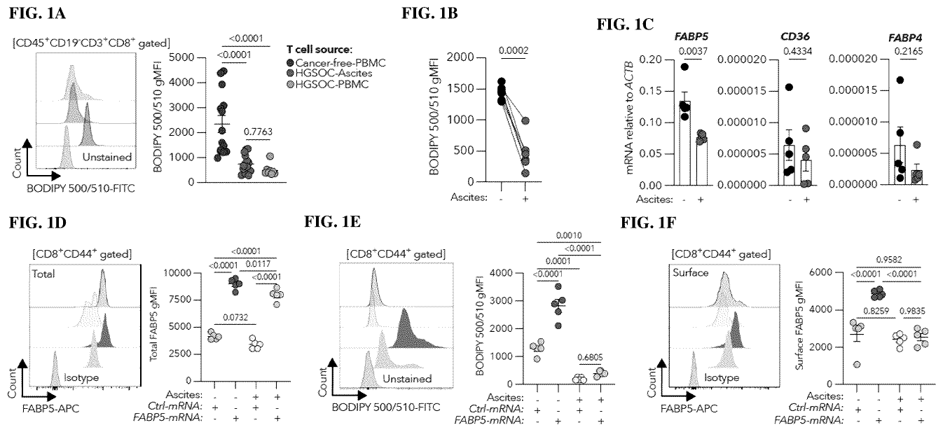

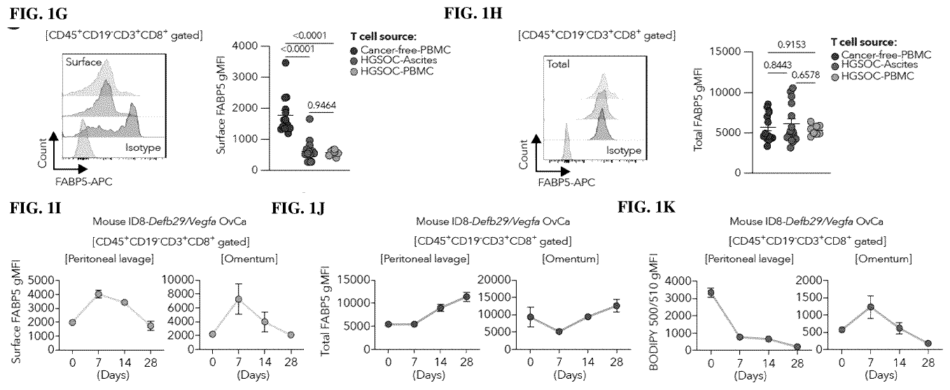

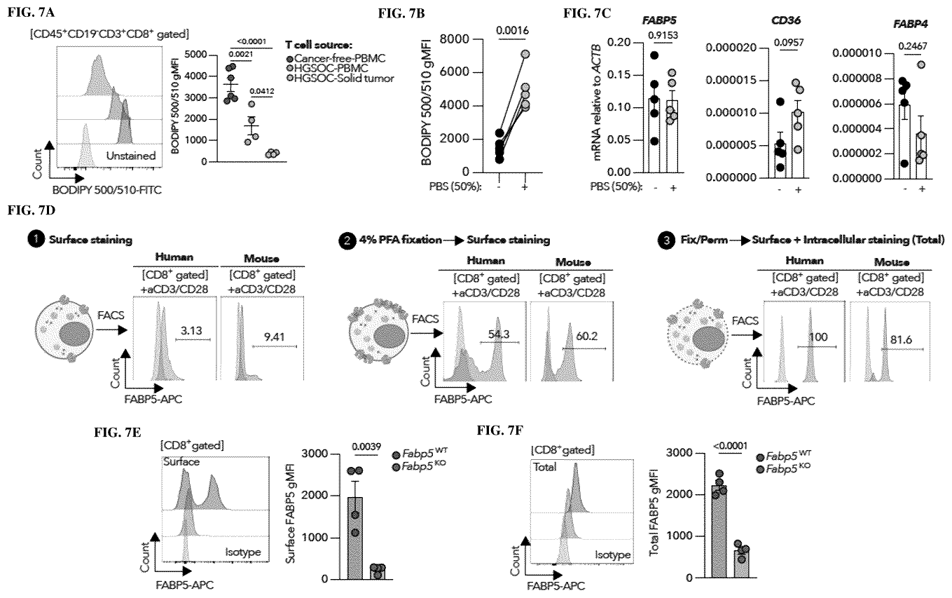

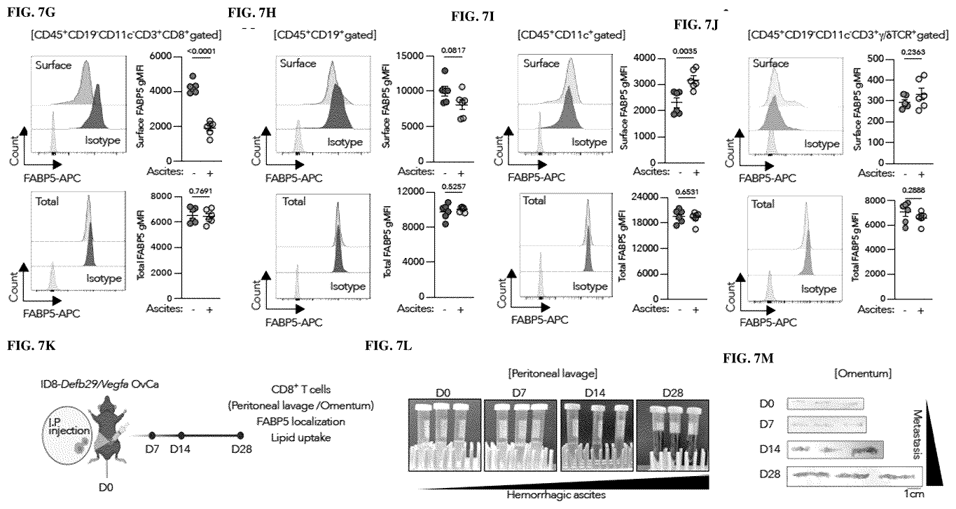

We sought to determine whether OvCa-infiltrating CD8⁺ T cells exhibit altered capacity to import extracellular fatty acids. FACS-based analyses using the fluorescent fatty acid analog C1-BODIPY 500/510 C12 showed that CD8⁺ T cells present in the ascites, solid tumors, or peripheral blood of patients with high-grade serous OvCa (HGSOC), the most common and aggressive form of OvCa²⁰, demonstrate defective lipid uptake compared with peripheral CD8⁺ T cells isolated from cancer-free women (FIG. 1A and FIG. 7A). Of note, in vitro exposure to HGSOC ascites supernatants markedly impaired extracellular fatty acid uptake by activated CD8⁺ T cells isolated from cancer-free donors (FIG. 1B), whereas exposure to control PBS enhanced fatty acid uptake (FIG. 7B). This ascites-induced defect was accompanied by a ˜50% reduction in FABP5 expression but not in genes encoding other lipid transporters such as CD36 and FABP4 (FIG. 1C and FIG. 7C). We therefore tested whether increasing FABP5 transcript levels could rescue this process. Intriguingly, FABP5 overexpression via transient mRNA electroporation augmented the total protein levels of this lipid transporter in activated CD8⁺ T cells exposed to HGSOC ascites supernatants (FIG. 1D) but failed to restore their fatty acid uptake capacity (FIG. 1E). Since FABP5 can be found in the cytosol or on the plasma membrane²¹, these results prompted us to investigate whether OvCa might alter the localization and/or activity of FABP5 to restrain extracellular lipid transport into CD8⁺ T cells.

We optimized a flow cytometry-based immunofluorescence staining method to discern the expression levels of surface-localized versus total FABP5 in human and mouse CD8⁺ T cells (FIG. 7D), which was validated using Fabp5 knockout mice (FIG. 7E, FIG. 7F). FABP5 mRNA supplementation significantly increased surface FABP5 expression when human CD8⁺ T cells were kept in normal culture media, but not upon exposure to HGSOC patient-derived ascites (FIG. 1F). Consistent with these in vitro findings, CD8⁺ T cells isolated from the ascites or peripheral blood of HGSOC patients demonstrated a significant reduction in surface FABP5 expression compared with peripheral CD8⁺ T cells from cancer-free women (FIG. 1G), although their total FABP5 levels were comparable (FIG. 1H). The decrease in FABP5 surface expression occurred in CD8⁺ T cells under ascites exposure, but not in other lymphocytes or myeloid cell populations (FIG. 7G-FIG. 7J).

We next evaluated the status of surface FABP5 expression and lipid uptake in tumor-associated CD8⁺ T cells throughout the development of metastatic OvCa. To this end, we used the orthotopic ID8-Defb29/Vegfa mouse model²² (FIG. 1K), which progressively generates an immunosuppressive peritoneal carcinomatosis characterized by ascites accumulation and omental metastases (FIG. 1L, FIG. 1M) that recapitulate the advanced stages of human OvCa²³⁻²⁷. Notably, surface FABP5 expression increased on CD8⁺ T cells in the peritoneal cavity and omentum during the first week of tumor progression, compared with their counterparts in naïve mice, but markedly declined thereafter as ascites and omental metastatic lesions developed (FIG. 1I). In sharp contrast, total FABP5 expression in the same CD8⁺ T cells analyzed increased throughout tumor progression (FIG. 1J). Consistent with the observed reduction in surface FABP5 expression, CD8⁺ T cells infiltrating these tumor locations also demonstrated major defects in fatty acid uptake over time (FIG. 1K). These results uncover that the ovarian tumor microenvironment disrupts the surface localization of FABP5 in CD8⁺ T cells, limiting their ability to import extracellular fatty acids.

### Example 3: TAGLN2 Enables FABP5-Driven Lipid Uptake in CD8⁺ T Cells

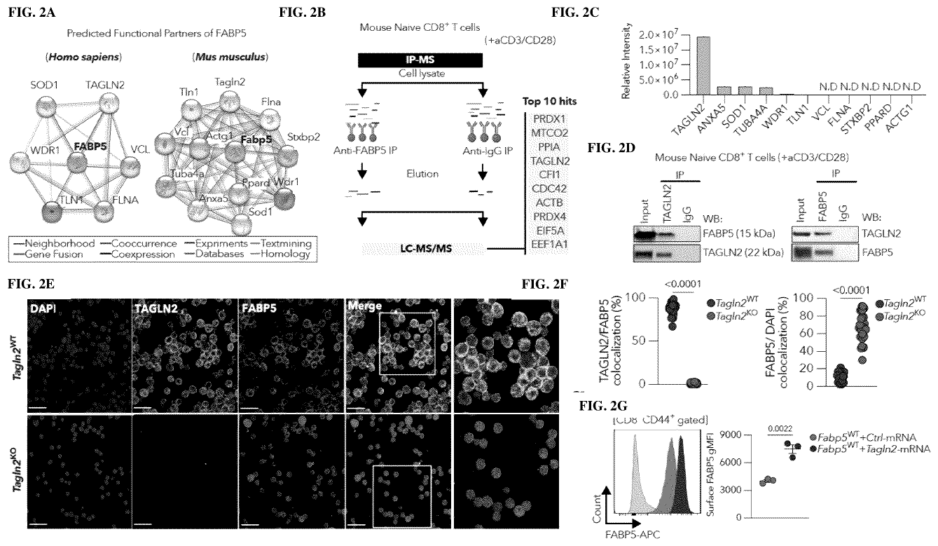

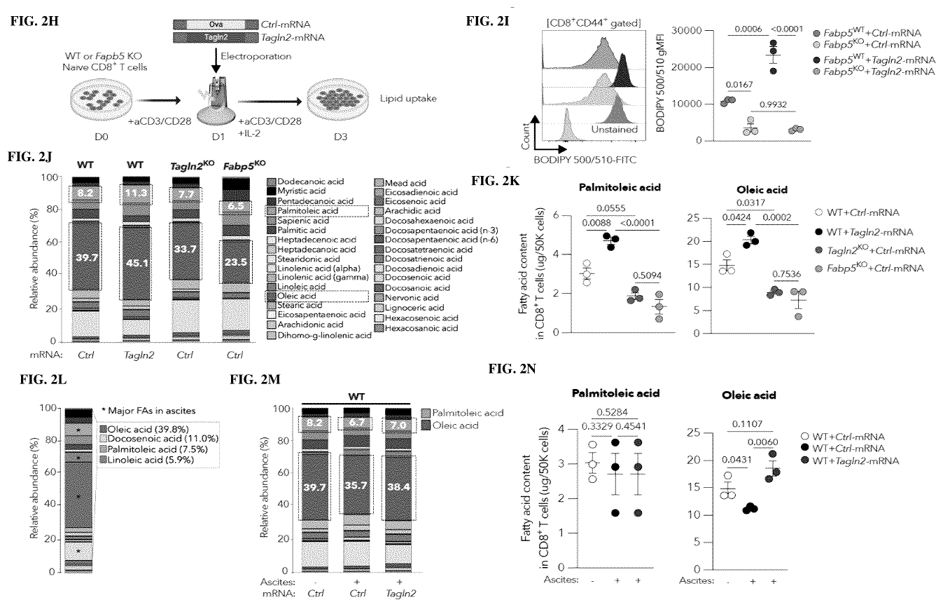

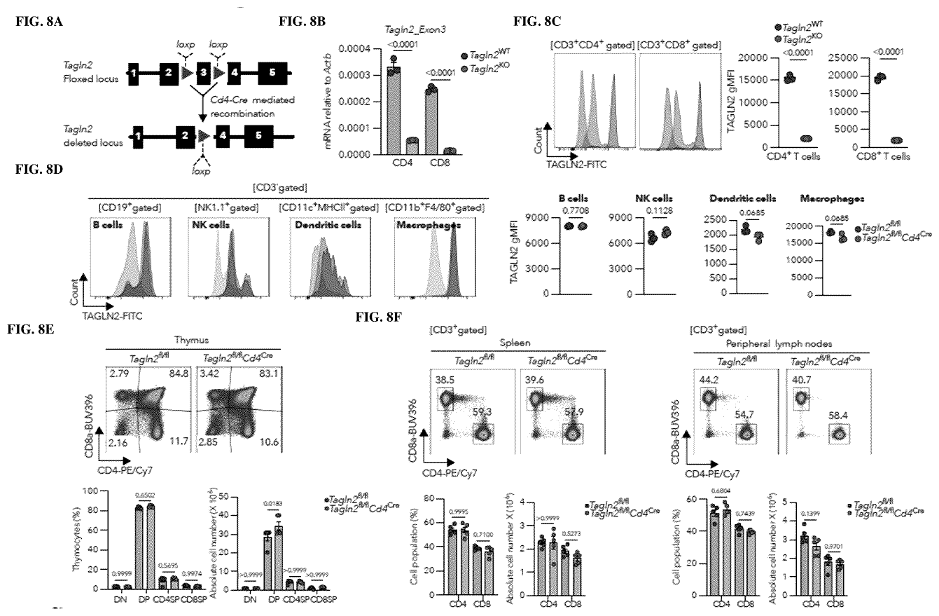

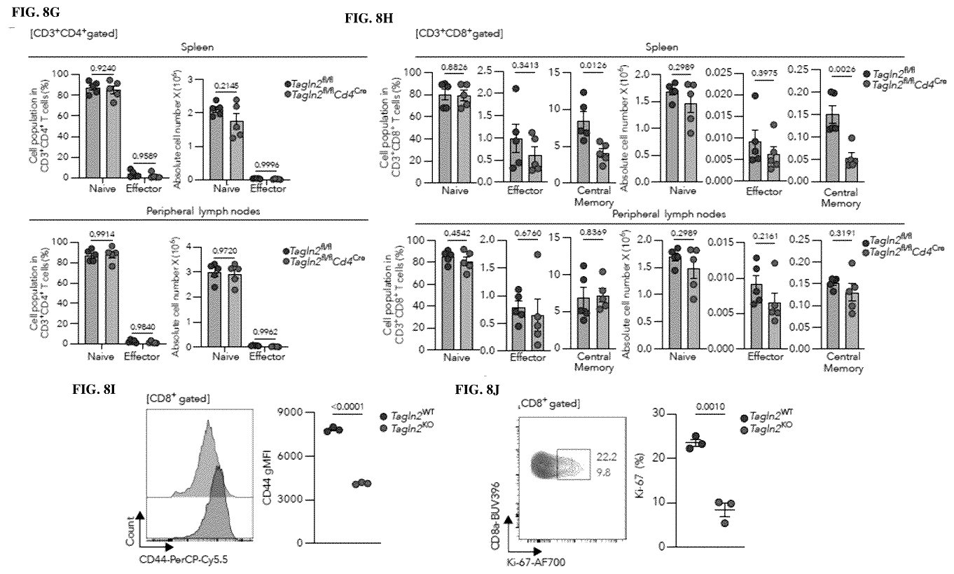

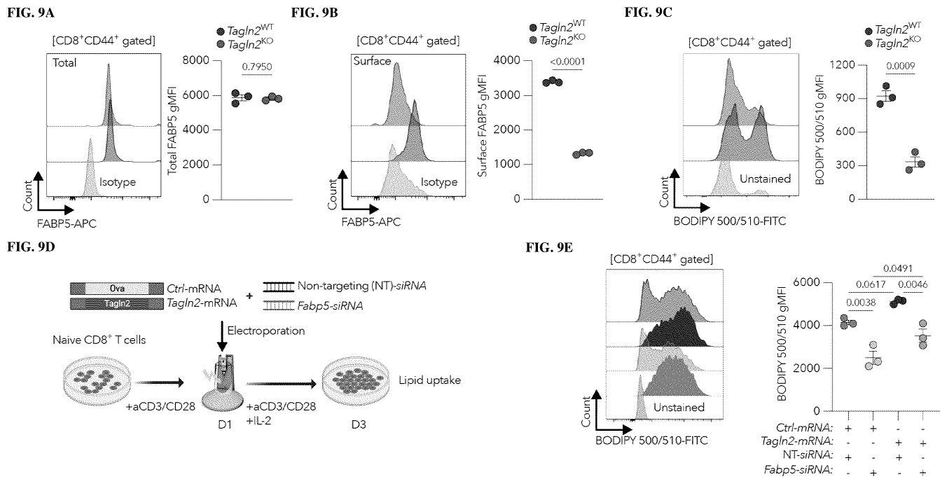

We sought to define the molecular mechanisms controlling FABP5 trafficking to the plasma membrane in activated CD8⁺ T cells. STRING database²⁸ analyses suggested that FABP5 interacts with multiple cytoskeletal proteins in humans and mice (FIG. 2A). Thus, we hypothesized that accessory cytoskeletal elements might guide FABP5 surface localization. We conducted unbiased immunoprecipitation assays followed by mass spectrometry (IP-MS) to identify FABP5-binding partners in mouse CD8⁺ T cells activated via CD3/CD28 (FIG. 2B). Amongst the FABP5-interacting proteins predicted by the STRING database, Transgelin 2 (TAGLN2) exhibited the highest representation after IP-MS analysis (FIG. 2C). Previous reports indicate that FABP5 and TAGLN2 coexist within macromolecular complexes conserved across multiple metazoans²⁹. Indeed, we confirmed the endogenous FABP5-TAGLN2 interaction in mouse activated CD8⁺ T cells through co-immunoprecipitation assays (FIG. 2D). TAGLN2 is an actin-binding protein that stabilizes actin structures and participates in multiple actin-associated signaling pathways^{30,31} This small (22-kDa) protein has been shown to promote lipid uptake and utilization in adipocytes³², and has been implicated in T cell activation, adhesion, migration, and effector function³³. Therefore, we hypothesized that TAGLN2 mediated FABP5 surface localization and fatty acid uptake in CD8⁺ T cells. We generated new transgenic mice where exon 3 of the Tagln2 gene was flanked by two loxp sites (Tagln2^{fl/fl}), and these mice were crossed with the Cd4^{Cre} strain to attain selective deletion of TAGLN2 in T cells (FIG. 8A-FIG. 8D). Tagln2^{fl/fl}Cd4^{Cre} mice did not exhibit gross abnormalities and showed normal proportions and numbers of thymocytes and peripheral CD4+ and CD8⁺ T cells, including their corresponding subsets (FIG. 8E-FIG. 8H). However, these conditional knockout mice exhibited a significant decrease in the proportion and absolute number of splenic CD62L^{high}CD44high central memory CD8⁺ T cells (FIG. 8H). Consistent with prior reports³³, TAGLN2-deficient CD8⁺ T cells stimulated via CD3/CD28 showed decreased expression of CD44 and Ki-67 (FIG. 8I, FIG. 8J). FABP5 and TAGLN2 co-localized to the plasma membrane in Tagln2^{fl/fl} CD8⁺ T cells undergoing activation, whereas FABP5 was mostly restricted to perinuclear locations in their TAGLN2-deficient counterparts (FIG. 2E, FIG. 2F). Tagln2 overexpression increased the expression of FABP5 on the surface of activated CD8⁺ T cells (FIG. 2G) without affecting the levels of total FABP5 (FIG. 9A). Accordingly, surface expression of FABP5 (FIG. 9B) and extracellular fatty acid uptake (FIG. 9C) were drastically reduced in activated TAGLN2-deficient CD8⁺ T cells, compared with their wild type (WT) counterparts. These data indicate that the surface localization and function of FABP5 as a major importer of extracellular fatty acids in CD8⁺ T cells depends on TAGLN2.

To further confirm these findings, WT or FABP5-deficient CD8⁺ T cells activated via CD3/CD28 were transfected with mRNAs encoding control ovalbumin (Ctrl-mRNA) or mouse TAGLN2 (Tagln2-mRNA), and their lipid uptake capacity was assessed thereafter (FIG. 2H). As expected, CD8⁺ T cells devoid of FABP5 showed lower lipid uptake than their WT counterparts (FIG. 2I). Notably, Tagln2 overexpression bolstered extracellular fatty acid import in WT but not FABP5-deficient CD8⁺ T cells (FIG. 2I), and similar effects were observed when Fabp5 was knocked-down in CD8⁺ T cells using siRNA (FIG. 9D, FIG. 9E). Collectively, these data establish that FABP5 and TAGLN2 functionally cooperate to mediate optimal lipid import in CD8⁺ T cells.

Lipidomic analyses determined that TAGLN2 overexpression in activated CD8⁺ T cells increased their intracellular levels of palmitoleic acid and oleic acid, which are normally imported by FABP5³⁴. In contrast, the abundance of these lipids significantly decreased in activated CD8⁺ T cells lacking either TAGLN2 or FABP5 (FIG. 2J, FIG. 2K). We further investigated whether the TAGLN2-FABP5 axis enabled CD8⁺ T cells to import fatty acids present in the OvCa ascites. We focused on oleic acid as it is overrepresented in this malignant fluid (FIG. 2L). Uptake of palmitoleic acid by CD8 T cells was not altered upon ascites exposure or TAGLN2 overexpression. However, the import of oleic acid was compromised in ascites-incubated CD8 T cells, and this defect was rescued upon TAGLN2 overexpression. (FIG. 2M, FIG. 2N). These data indicate that oleic acid is imported by the TAGLN2-FABP5 axis in either normal or OvCa-exposed CD8⁺ T cells.

### Example 4: Dysfunctional CD8⁺ T Cells Infiltrating OvCa Lack TAGLN2

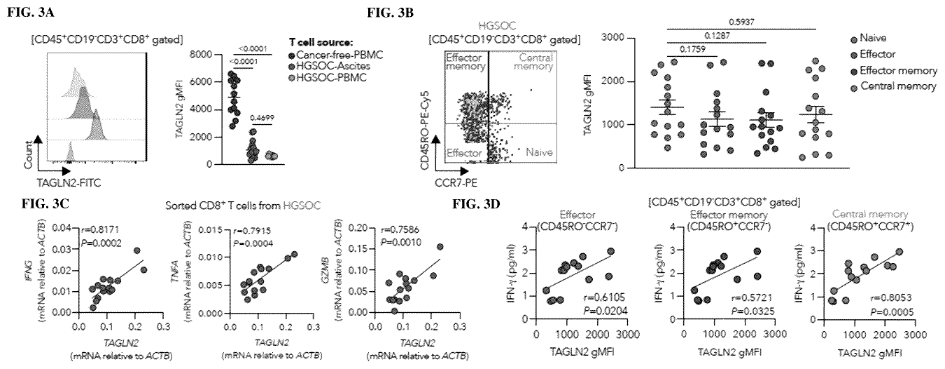

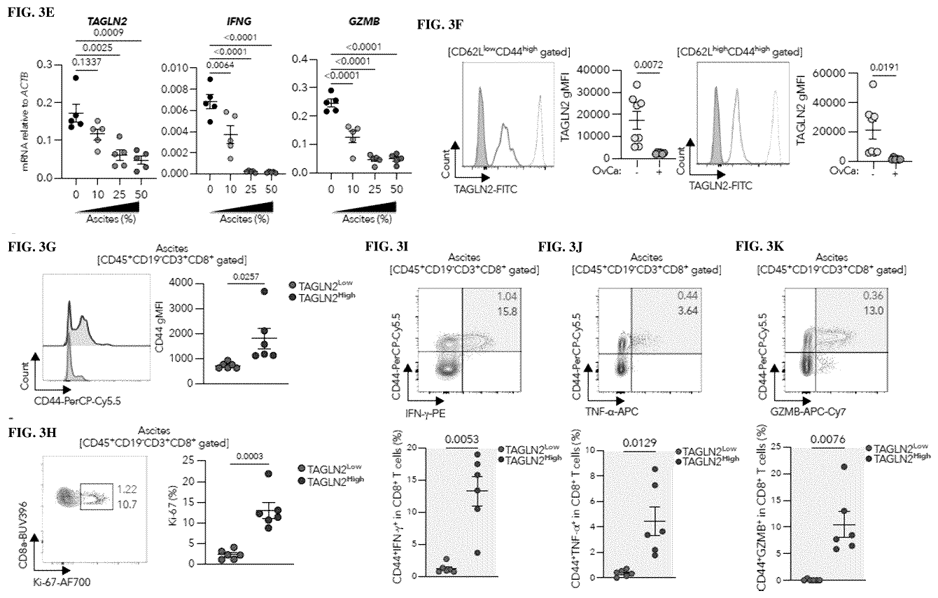

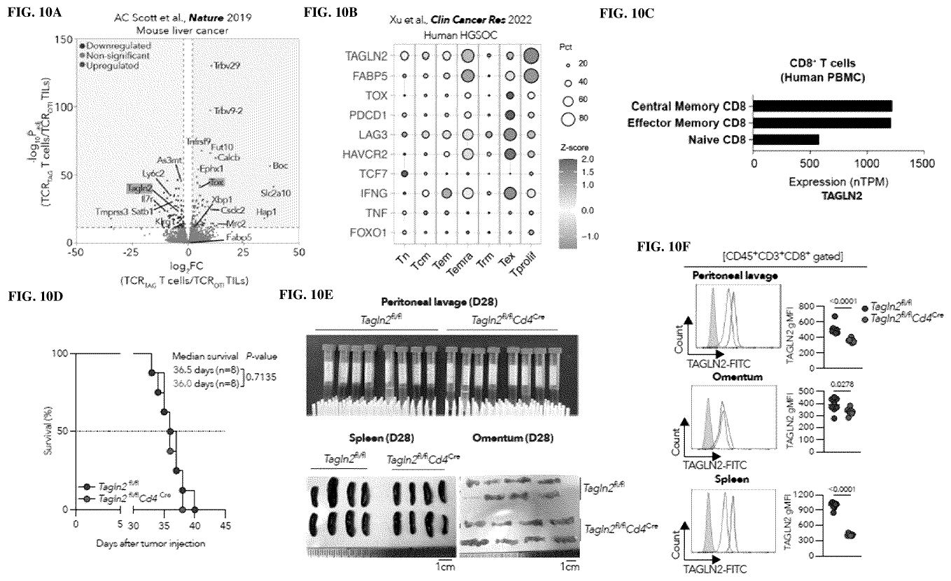

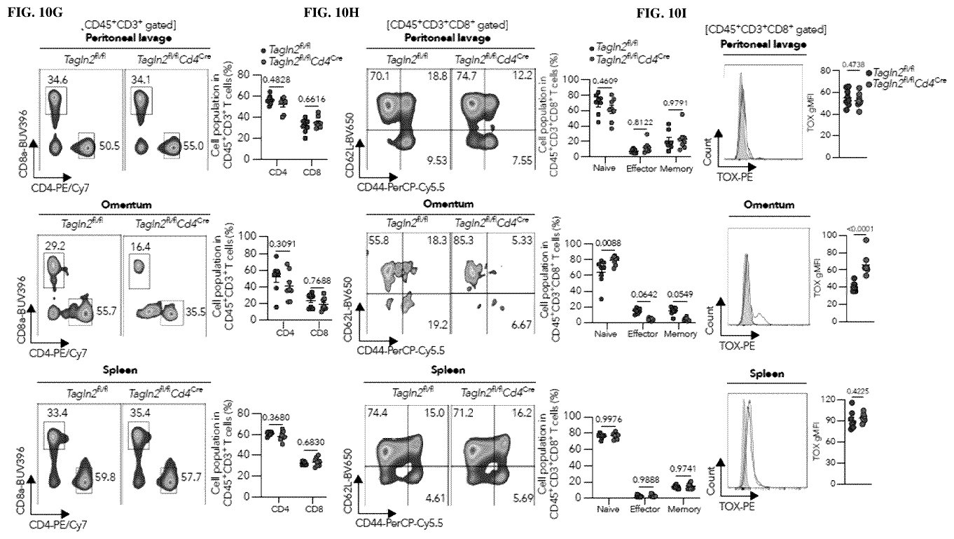

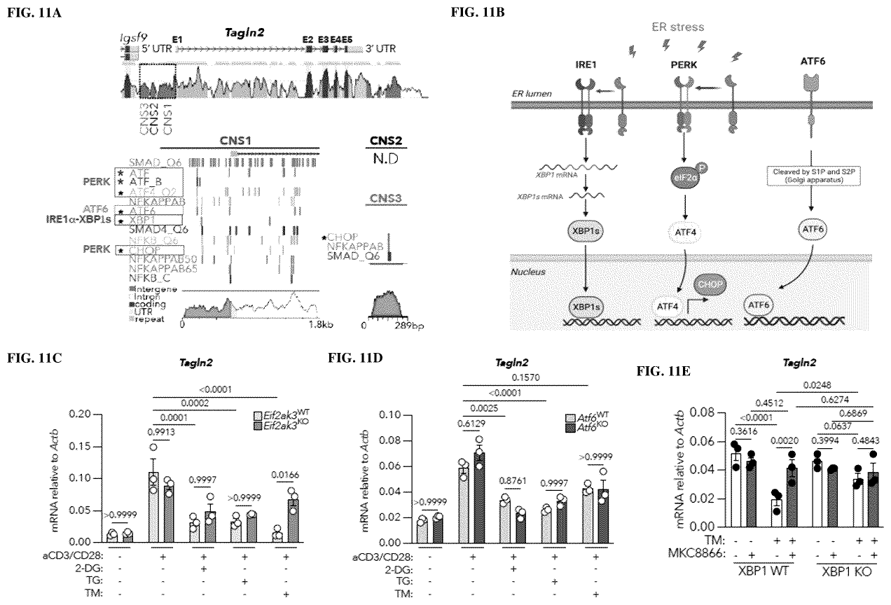

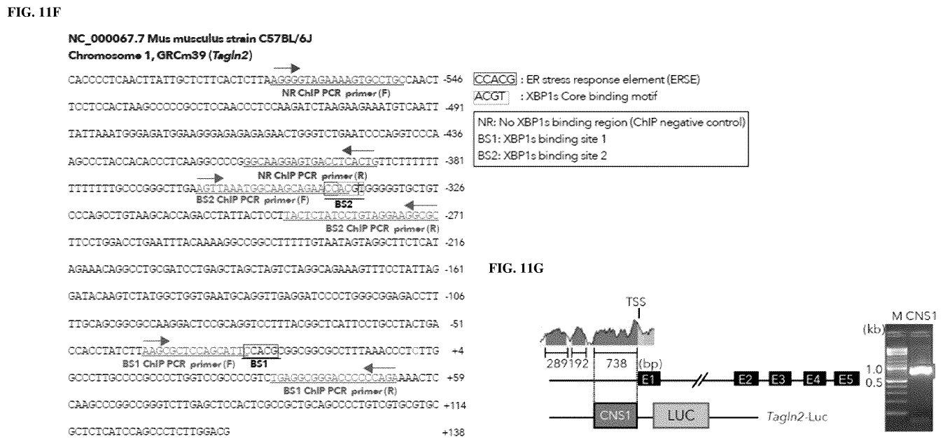

TAGLN2 has been implicated in T cell cytokine production and effector capacity in vitro³³. Moreover, Tagln2 is upregulated in functionally competent effector and memory CD8⁺ T cell subsets during viral infection^{35,36}. Whether tumors silence TAGLN2 in infiltrating T cells to alter their lipid metabolism and protective activity is unknown. We analyzed previously published gene expression datasets^{37,38} and found marked TAGLN2 downregulation in dysfunctional and exhausted CD8⁺ T cells infiltrating murine hepatocellular carcinoma and human HGSOC (FIG. 10A, FIG. 10B). Furthermore, we found that the intracellular levels of TAGLN2 were significantly lower in CD8⁺ T cells isolated from the ascites or peripheral blood of HGSOC patients than in peripheral CD8⁺ T cells from cancer-free women (FIG. 3A). While activated T cells normally express higher TAGLN2 than their naïve counterparts (FIG. 10C), effector and memory T cells present in the ascites of HGSOC patients demonstrated low TAGLN2 expression that was comparable to that of naïve T cells in the same microenvironment (FIG. 3B). The levels of TAGLN2 positively correlated with the intrinsic expression of IFNG, TNF, and GZMB transcripts in CD8⁺ T cells residing in the ascites of HGSOC patients (FIG. 3C). Furthermore, the amount of TAGLN2 in effector and memory CD8⁺ T cell subsets in this malignant fluid correlated with the local concentration of IFN-γ (FIG. 3D). Exposure to cell-free ascites supernatants from HGSOC patients drastically suppressed TAGLN2, IFNG, and GZMB expression in pre-activated CD8⁺ T cells obtained from peripheral blood of cancer-free women (FIG. 3E). In the ascites of mice with metastatic ID8-Defb29/Vegfa OvCa, effector (CD62L^{low}CD44^{high}) and central memory (CD62L^{high}CD44^{high}) CD8⁺ T cells demonstrated a marked reduction in TAGLN2 expression, compared with the same T cell subsets in peritoneal lavage from cancer-free mice (FIG. 3F). Notably, in this model, ascites-resident TAGLN2high CD8⁺ T cells demonstrated higher effector capacity than their TAGLN2^{low} counterparts as evidenced by superior expression of CD44, Ki-67, IFN-γ, TNF-α, and Granzyme B (FIG. 3G-FIG. 3K). We developed metastatic ID8-Defb29/Vegfa OvCa in WT (Tagln2^{fl/fl}) or T cell-specific TAGLN2-deficient (Tagln2^{fl/fl}Cd4^{Cre}) mice and found no major differences in their overall survival rates, tumor progression, and proportion of intratumoral T cell subsets (FIG. 10D-FIG. 10I), consistent with the drastic loss of TAGLN2 in tumor-associated T cells from early stages of disease progression (FIG. 10F). These results indicate that OvCa potently inhibits TAGLN2 expression in infiltrating CD8⁺ T cells and suggest that early loss of this cytoskeletal element is linked to intratumoral T cell malfunction.

### Example 5: ER Stress Responses Suppress TAGLN2 Expression in CD8⁺ T Cells

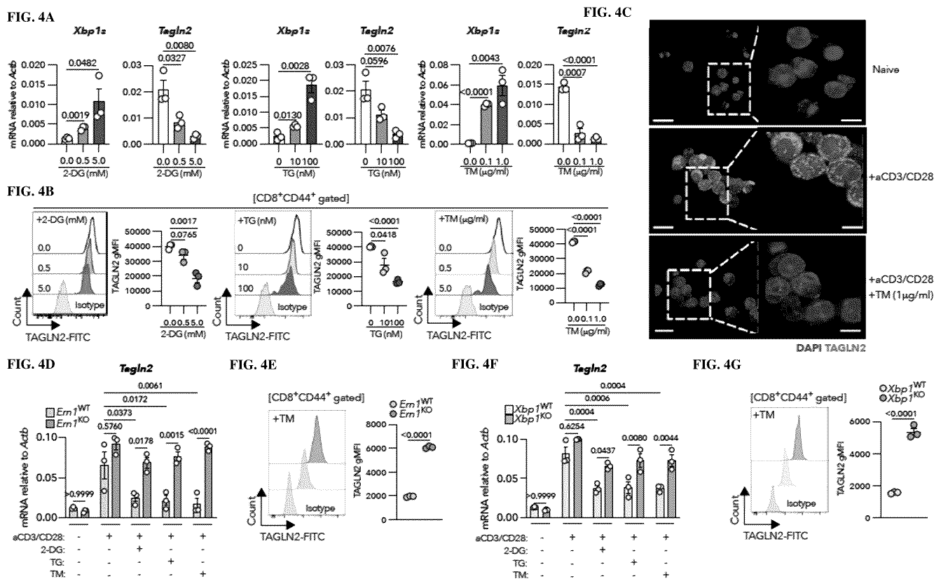

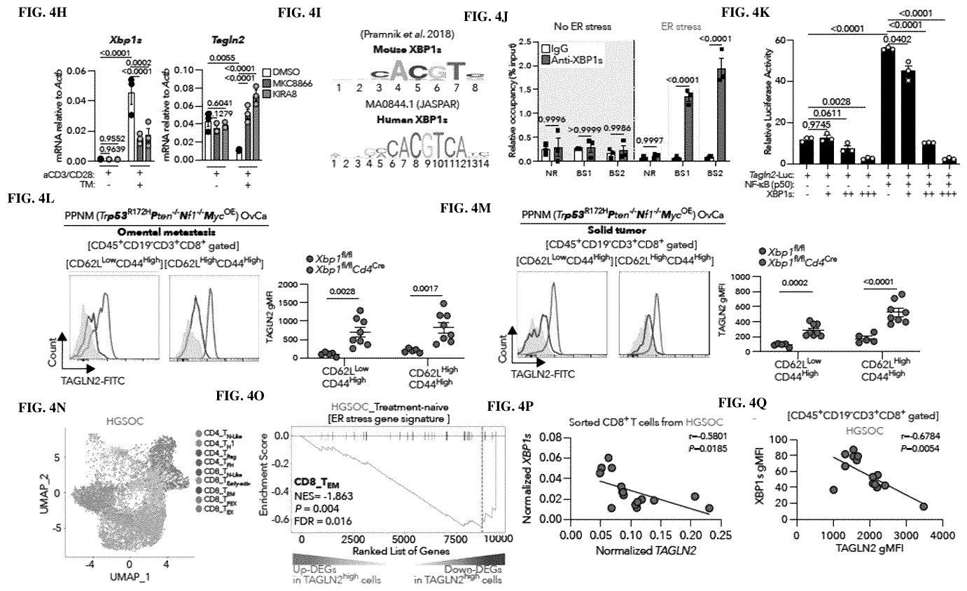

We sought to define the molecular mechanisms mediating TAGLN2 repression in OvCa-infiltrating CD8⁺ T cells. We identified three conserved noncoding sequences (CNS1, CNS2, and CNS3) within the 5′ promoter region of the Tagln2 locus that were highly associated with transcription factor binding sites and other cis-acting regulatory elements (FIG. 11A). Intriguingly, CNS1 (−738 to +134) contained multiple putative binding sites for transcription factors governing the endoplasmic reticulum (ER) stress response, including XBP1s, ATF4, and ATF6 (FIG. 11A). These elements are induced upon activation of the ER-resident stress sensors inositol-requiring enzyme-1α (IRE1α), protein kinase RNA-like endoplasmic reticulum kinase (PERK), and activating transcription factor 6 (ATF6), respectively³⁹ (FIG. 11B). Since dysregulated ER stress responses promote T cell malfunction and immune escape in diverse tumor types, including OvCa^{17,18,40}, we evaluated whether ER stress-inducing conditions altered Tagln2 expression in activated CD8⁺ T cells. Exposure to classical ER stressors such as 2-Deoxy-D-glucose (2-DG), thapsigargin (TG), or tunicamycin (TM) induced the canonical ER stress response marker Xbp1s while markedly decreasing the expression of Tagln2 (FIG. 4A). FACS analyses confirmed reduced intracellular TAGLN2 levels in activated CD8⁺ T cells experiencing ER stress (FIG. 4B), and these results were further validated by confocal microscopy (FIG. 4C).

To determine the arms of the ER stress response mediating Tagln2 repression, similar experiments were conducted using transgenic CD8⁺ T cells independently lacking IRE1α, PERK, or ATF6. Signaling through PERK or ATF6 did not play a major role in regulating Tagln2 expression in ER-stressed CD8⁺ T cells (FIG. 11C, FIG. 11D). In contrast, IRE1α-deficient CD8⁺ T cells facing ER stress failed to downregulate Tagln2 (FIG. 4D) and maintained higher TAGLN2 protein expression (FIG. 4E) than their WT counterparts under the same condition. Upon activation, IRE1α excises a 26-nucleotide fragment from the Xbp1 mRNA, generating a spliced isoform that codes for the functionally active transcription factor XBP1s⁴¹ (FIG. 11B). Notably, XBP1s-deficient CD8⁺ T cells also preserved Tagln2 transcription and protein expression under ER stress (FIG. 4F, FIG. 4G). Treatment with the selective IRE1α pharmacological inhibitors MKC8866⁴² or KIRA8⁴³, which prevent XBP1s generation, also enhanced Tagln2 expression in activated CD8⁺ T cells undergoing ER stress (FIG. 4H). Furthermore, MKC8866 treatment did not alter Tagln2 expression in XBP1-deficient CD8⁺ T cells undergoing ER stress, indicating that the Tagln2 mRNA is not a substrate of regulated IRE1α-dependent decay (RIDD) (FIG. 11E) and that XBP1s may repress its normal transcription. Hence, the IRE1α-XBP1s arm of the ER stress response operates as a dominant negative regulator of TAGLN2 expression in activated CD8⁺ T cells.

XBP1s is a multitasking transcription factor that controls gene expression in a cell-specific and context-dependent manner^{40,44}. We examined whether XBP1s could bind the Tagln2 promoter to alter its transcription. Confirming our initial analysis (FIG. 11A), putative XBP1s core binding motif and ER stress response element (ERSE) sequences were found in the Tagln2 promoter (FIG. 4I and FIG. 11F). Chromatin immunoprecipitation (ChIP) followed by PCR (ChIP-PCR) experiments demonstrated robust XBP1s binding to the promoter regions identified only in pre-activated CD8⁺ T cells experiencing ER stress (FIG. 4J). Interestingly, CNS1 also contained multiple binding motifs for NF-kB (FIG. 11A), which had been shown to act as a positive regulator of Tagln2 expression in macrophages⁴⁵. Thus, we used luciferase reporter assays (FIG. 11G) to test whether XBP1s inhibited NF-kB-directed transcription of Tagln2. Potent luciferase reporter activity driven by the Tagln2 promoter was observed upon NF-kB expression (FIG. 4K). Yet, this transactivation was significantly and dose-dependently impaired upon introduction of XBP1s-encoding plasmids (FIG. 4K). These data uncover ER stress-induced XBP1s as a transcriptional repressor of Tagln2.

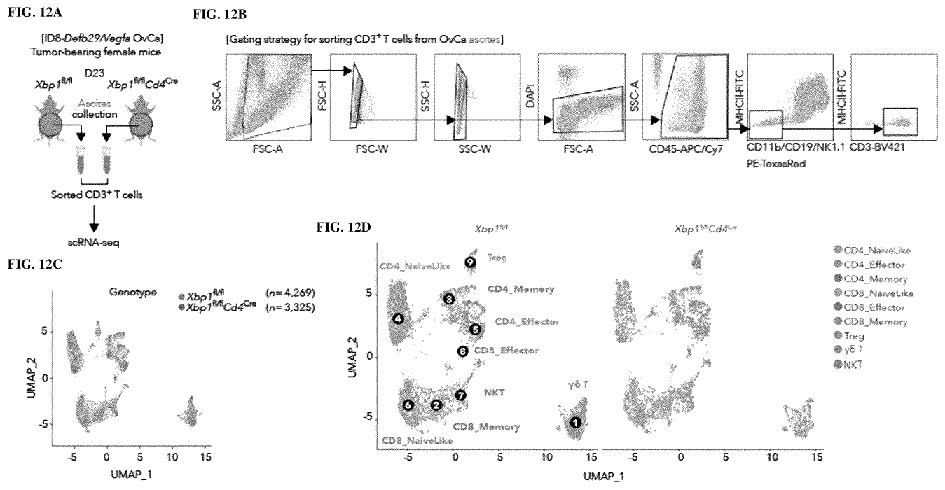

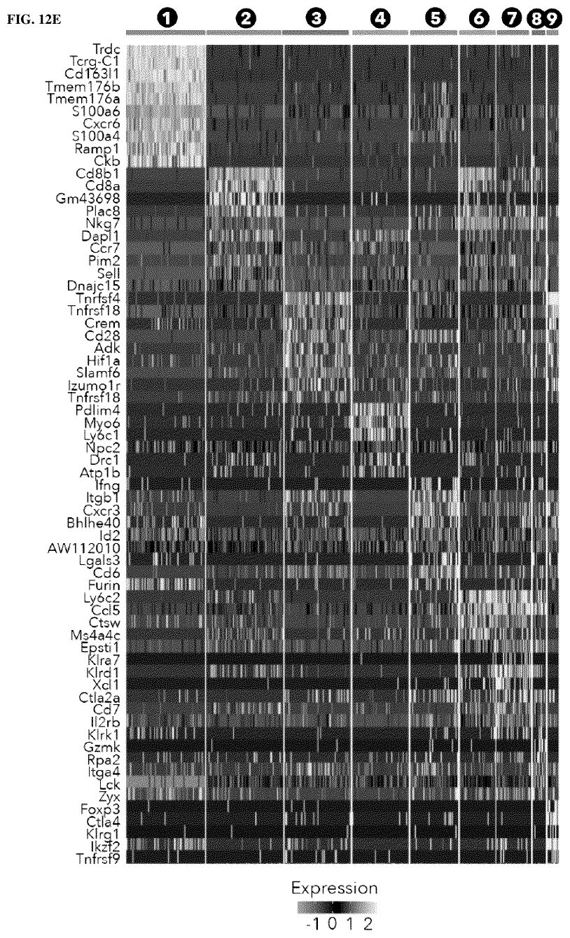

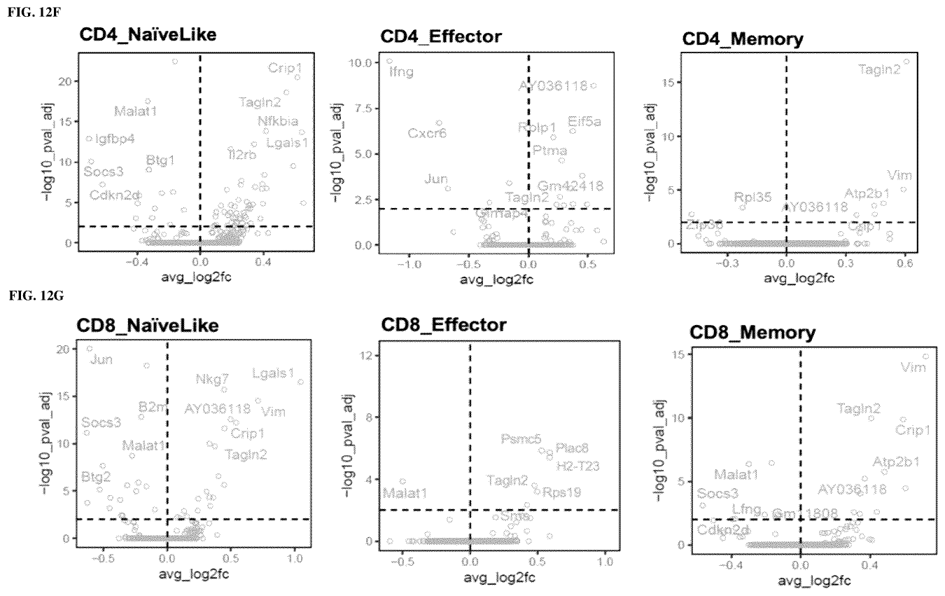

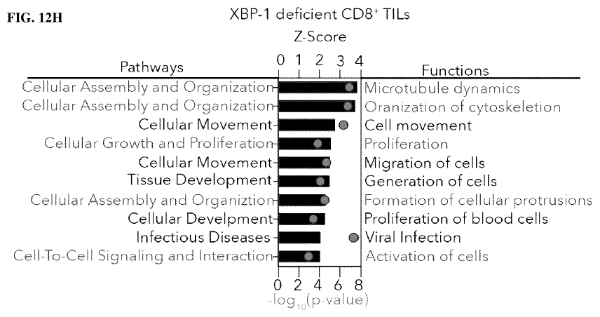

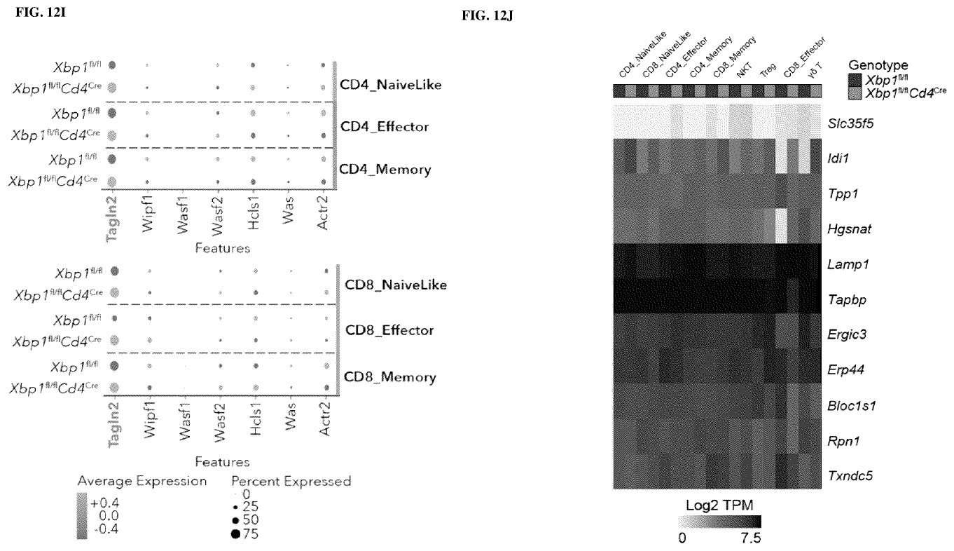

We next evaluated whether IRE1α-XBP1s signaling regulates Tagln2 expression in OvCa-infiltrating CD8⁺ T cells experiencing pathological ER stress in the tumor microenvironment¹⁷. To this end, we performed single-cell RNA sequencing (scRNA-seq) analyses of total CD45⁺CD3⁺ T cells sorted from the ascites of Xbp1^{fl/fl} or Xbp1^{fl/fl}Cd4^{Cre} mice bearing advanced ID8-Defb29/Vegfa OvCa (FIG. 12A-FIG. 12E). Tagln2 was significantly upregulated in multiple ascites-infiltrating CD4+ and CD8⁺ T cell subsets lacking XBP1s, compared with their WT counterparts (FIG. 12F, FIG. 12G). Of note, downstream cellular functions such as microtubule dynamics, organization of cytoskeleton, and proliferation and activation of cells, were predicted to be enhanced in ascites-infiltrating CD8⁺ T cells lacking XBP1s that maintain superior Tagln2 expression (FIG. 12H). When compared to other key cytoskeletal elements, only Tagln2 exhibited a distinct increase in various ascites-infiltrating CD4+ and CD8⁺ T cell subsets devoid of XBP1s (FIG. 12I), whereas canonical signs of RIDD were not observed in this setting (FIG. 12J).

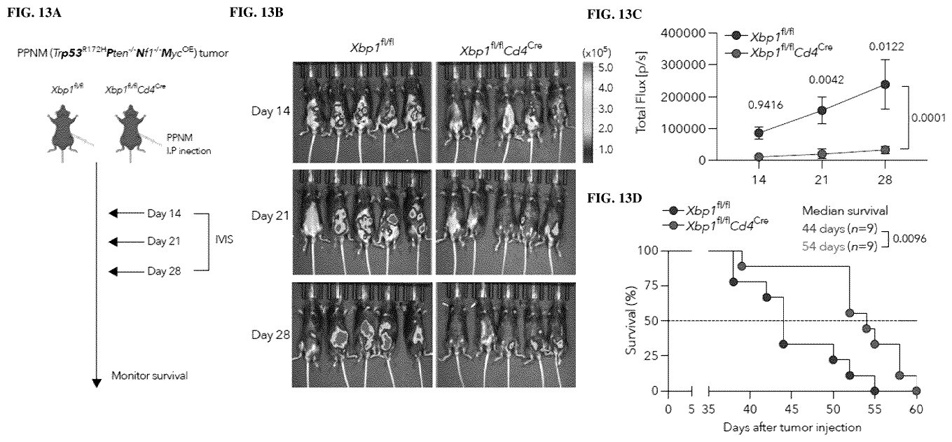

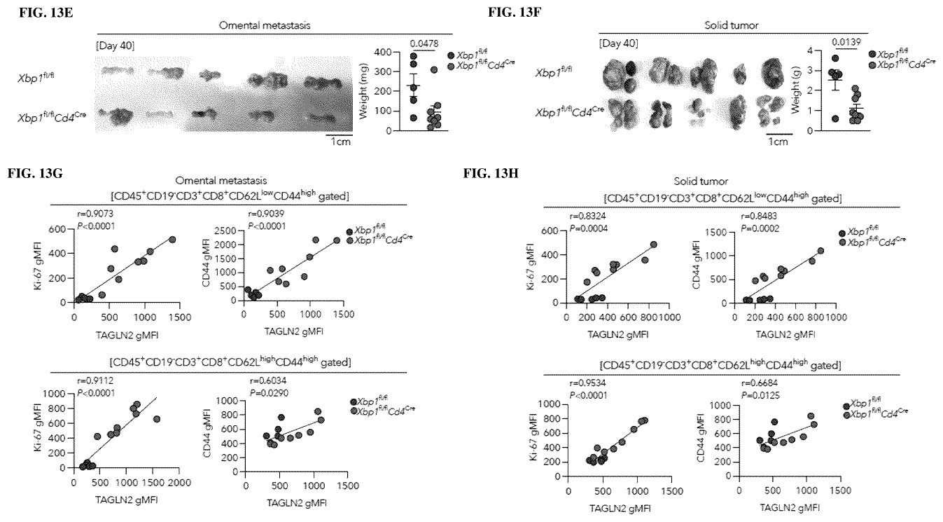

To confirm these results, we used the PPNM model of high-grade serous tubo-ovarian cancer (HGSC) that encompasses the most common genetic abnormalities observed in human HGSOC⁴⁶. Consistent with our prior findings¹⁷, T cell-intrinsic XBP1s also facilitated metastatic OvCa progression in this independent tumor system (FIG. 13A-FIG. 13F). Furthermore, XBP1s-deficient effector (CD62L^{low}CD44^{high}) and central memory (CD62L^{high}CD44high) CD8⁺ T cells infiltrating PPNM-derived omental and peritoneal tumors demonstrated higher expression of TAGLN2, Ki-67, and CD44 than their XBP1s-sufficient (Xbp1^{fl/fl}) counterparts (FIG. 4L, FIG. 4M and FIG. 13G, FIG. 13H).

We conducted gene-set enrichment analyses (GSEA) of scRNA-seq data generated from patient-derived HGSOC specimens⁴⁷ to further validate the findings. TAGLN2high effector memory CD8⁺ T cells infiltrating these human tumors demonstrated reduced expression of multiple ER stress response gene markers, including XBP1 and its canonical target genes (FIG. 4N, FIG. 4O). Notably, TAGLN2 mRNA and protein expression inversely correlated with the intrinsic levels of XBP1s in CD8⁺ T cells residing in the ascites of HGSOC patients (FIG. 4P, FIG. 4Q). Taken together, these data indicate that ER stress-driven IRE1α-XBP1s blunts TAGLN2 expression in intratumoral CD8⁺ T cells to enforce their dysfunctional state.

### Example 6: TAGLN2 Overexpression Enhances Lipid Uptake and Mitochondrial Respiration in ER-Stressed CD8⁺ T Cells

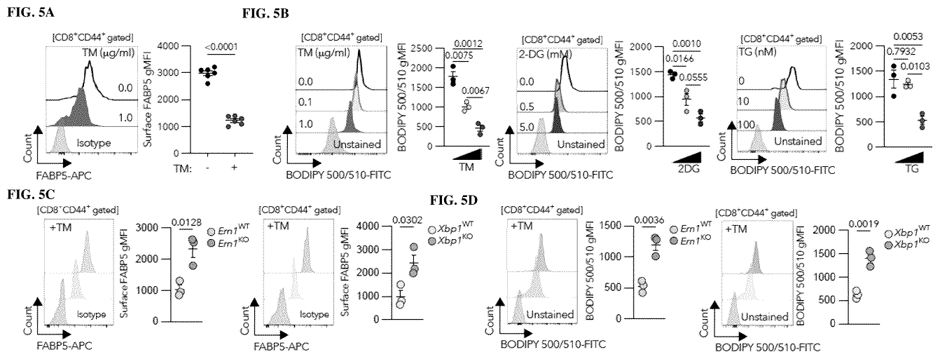

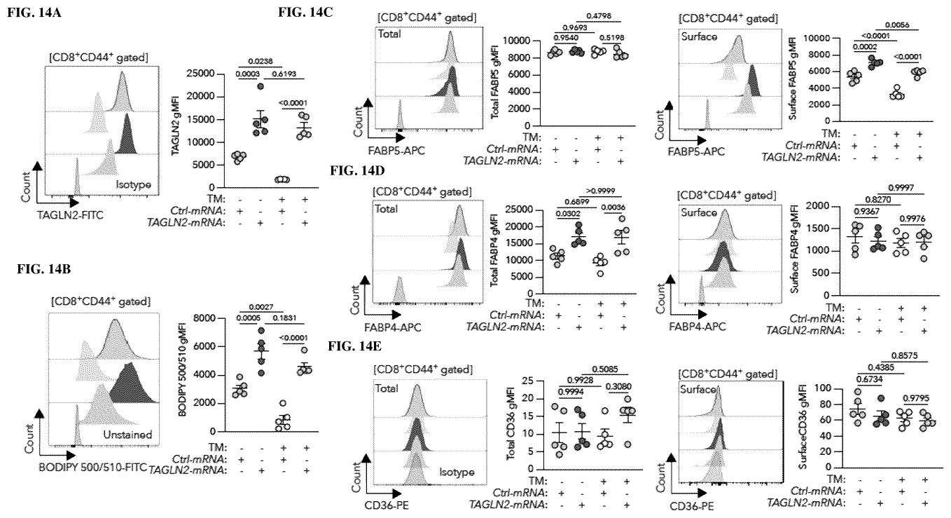

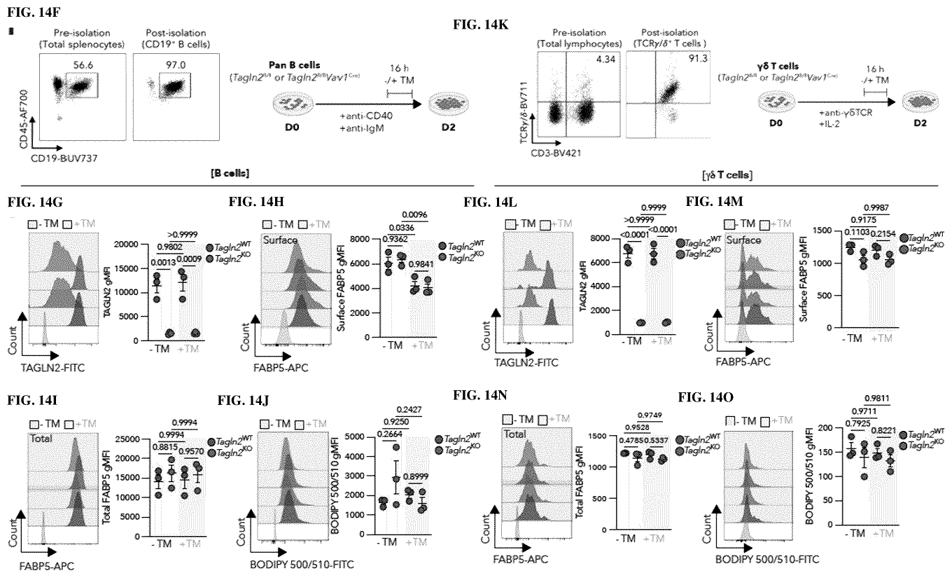

Extracellular fatty acids imported by transporters such as FABP5 are catabolized via fatty acid β-oxidation (FAO) to fuel mitochondrial respiration and generate ATP⁴⁸. We hypothesized that ER stress-driven repression of TAGLN2 disrupted the FABP5-FAO bioenergetic axis. Pre-activated CD8⁺ T cells treated with the ER stressor tunicamycin (TM) showed diminished FABP5 surface expression (FIG. 5A). Consistently, CD8⁺ T cells exposed to diverse ER stressors demonstrated impaired lipid uptake capacity compared with their non-stressed counterparts (FIG. 5B). IRE1α- or XBP1s-deficient CD8⁺ T cells facing ER stress demonstrated superior surface expression of FABP5 (FIG. 5C) and enhanced lipid uptake capacity compared with their WT counterparts under the same condition (FIG. 5D), confirming that IRE1α-XBP1s acts as an upstream regulator of TAGLN2-dependent FABP5 surface localization and lipid import. CD8⁺ T cells undergoing ER stress demonstrated a dose-dependent decrease in their basal and maximal oxygen consumption rates (FIG. 5E). We therefore tested whether increasing Tagln2 levels via mRNA electroporation could restore the bioenergetic defects caused by ER stress. Strikingly, Tagln2-rescued CD8⁺ T cells facing ER stress demonstrated enhanced lipid uptake (FIG. 5F), mitochondrial respiration (FIG. 5G), and expression of CD44, Ki-67, IFN-γ, and Granzyme B (FIG. 5H-FIG. 5J), compared with their counterparts transfected with a control mRNA encoding ovalbumin. Tagln2 overexpression rescued lipid uptake and FABP5 surface expression in activated CD8⁺ T cells undergoing ER stress (FIG. 14A, FIG. 14C) without affecting the surface localization of other lipid transporters such as FABP4 or CD36 (FIG. 14D, FIG. 14E). TAGLN2-dependent localization of FABP5 was not evident in B cells or 76 T cells (FIG. 14F-FIG. 14O), suggesting that the TAGLN2-FABP5 axis is selectively regulated by ER stress in CD8⁺ T cells. Treatment with the CPT1α inhibitor etomoxir rendered Tagln2-overexpressing CD8⁺ T cells unable to enhance mitochondrial respiration under ER stress (FIG. 5K), confirming that FAO mediates these effects. Exposure to oleic acid, one of the most abundant free-fatty acids in the ascites of HGSOC patients⁴⁹, bolstered CD44 and Ki-67 expression in ER-stressed CD8⁺ T cells overexpressing Tagln2 (FIG. 5L), denoting improved usage of these extracellular lipids to support their effector programs. Thus, we next tested whether TAGLN2 enabled the use of these fatty acids to sustain mitochondrial activity in activated CD8⁺ T cells experiencing glucose restriction, which is a prevalent condition in the tumor milieu. In the absence of exogenous oleic acid, glucose deprivation drastically reduced the mitochondrial membrane potential of CD8⁺ T cells irrespective of TAGLN2 status (FIG. 5M). Notably, oleic acid supplementation fully restored the mitochondrial membrane potential of WT but not TAGLN2-deficient CD8 T cells under glucose restriction (FIG. 5M). Hence, we conclude that ER stress blunts CD8⁺ T cell mitochondrial respiration by suppressing TAGLN2-mediated fatty acid uptake and oxidation. In addition, TAGLN2 promotes CD8⁺ T cell metabolic fitness by enabling the import and utilization of extracellular fatty acids that sustain mitochondrial activity in the absence of primary carbon sources such as glucose.

### Example 7: Preserving TAGLN2 Improves Cellular Immunotherapy in Metastatic OvCa

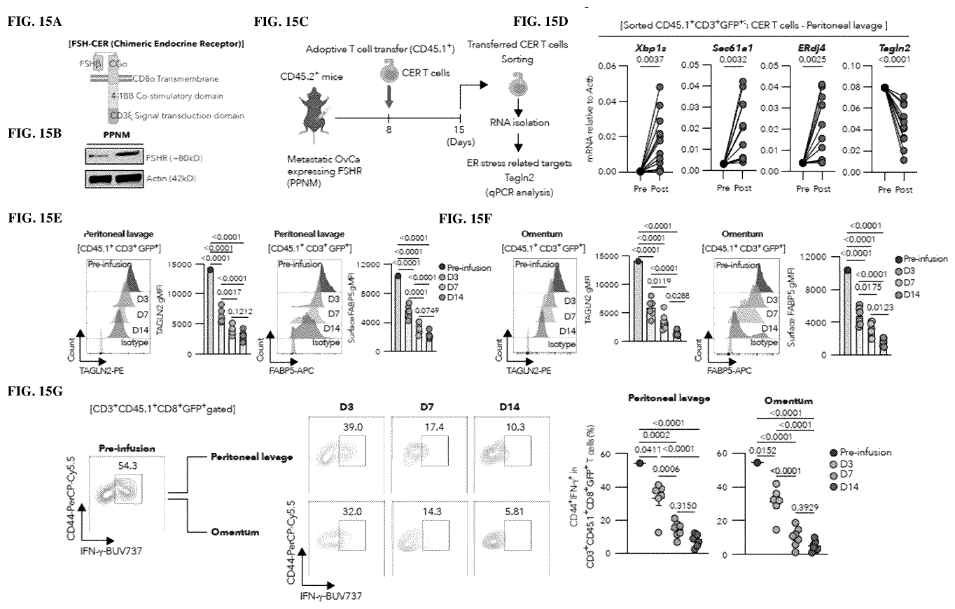

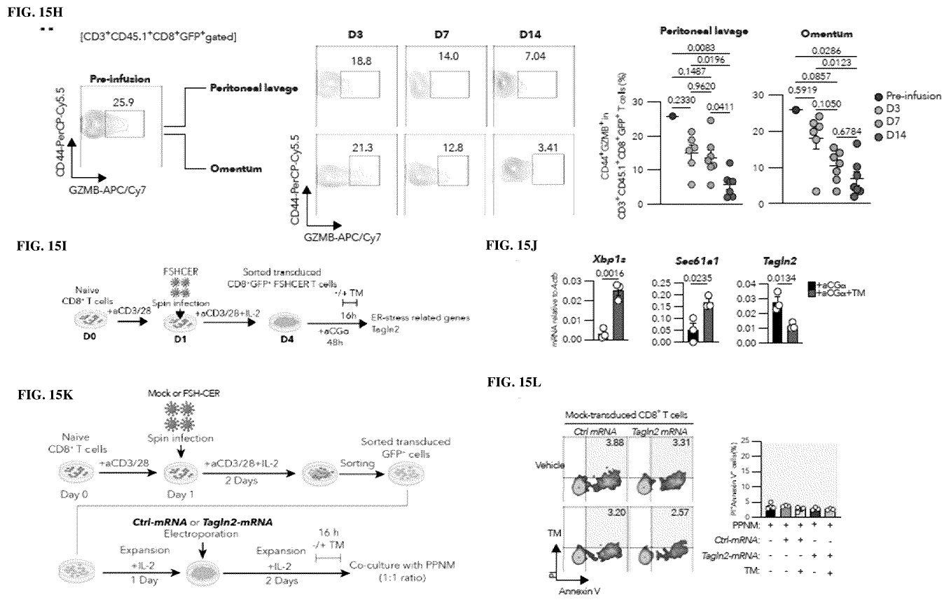

Adoptive T cell immunotherapies, including chimeric antigen receptor (CAR) T cells, have shown limited success against solid malignancies, especially OvCa¹⁴,1⁶. We hypothesized that tumor-induced suppression of TAGLN2 limits the therapeutic efficacy of CAR T cells in hosts with metastatic OvCa. To test this, we exploited T cells expressing chimeric endocrine receptors (CERs) that use the two subunits of the follicle-stimulating hormone (FSH) to target and kill FSH-receptor positive (FSHR⁺) ovarian tumors⁵⁰. In this system, T cells are transduced with a vector that encodes a chimeric receptor of the full-length of the β subunit of the FSH hormone, linked by a glycine/serine spacer, in frame with a CD8α transmembrane domain, the intracellular domain of co-stimulatory 4-1BB, and CD3ζ (FIG. 15A)⁵⁰. Importantly, these CER T cells are undergoing clinical testing in patients with advanced OvCa (NCT05316129).

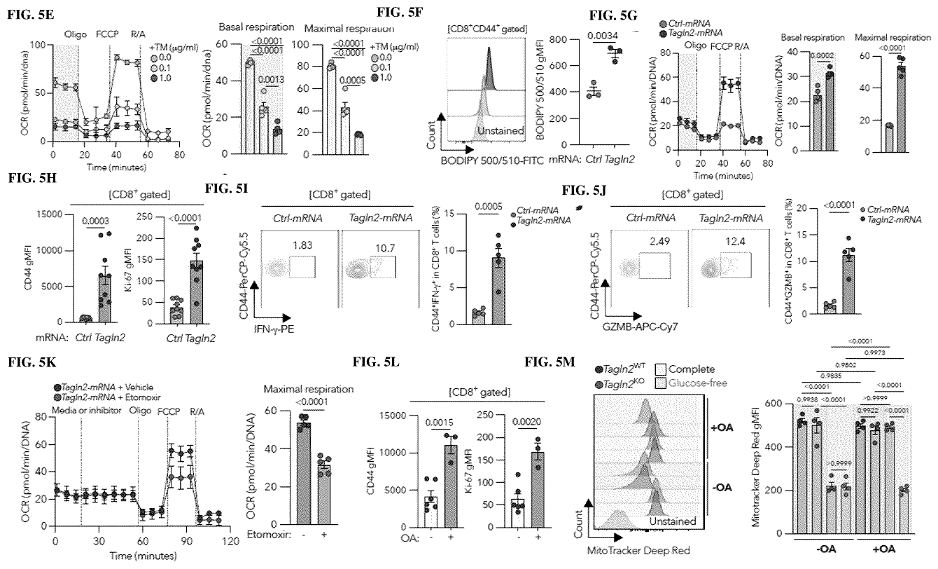

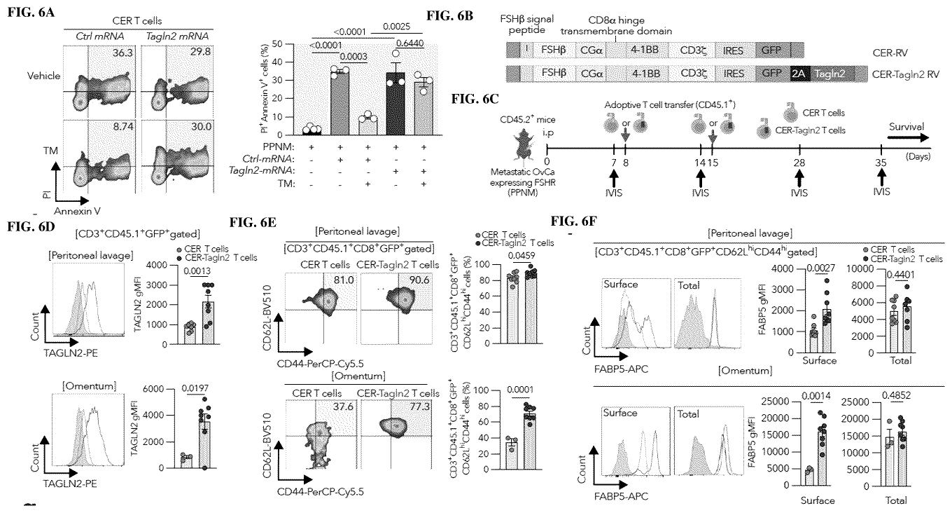

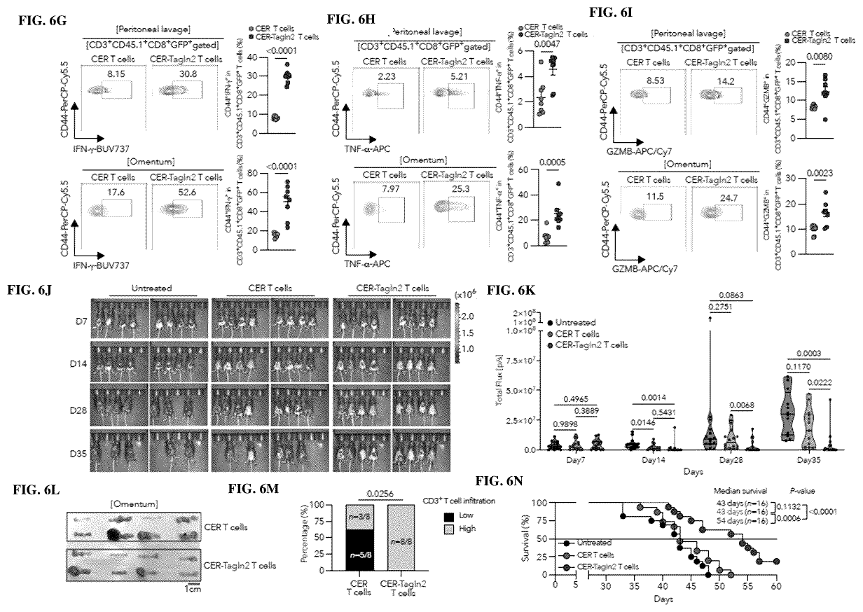

Female mice developing PPNM-based ovarian tumors that inherently express the FSHR (FIG. 15B) were intraperitoneally infused with CER T cells, and the transferred T cells were sorted from tumor locations 7 days later based on the differential expression of congenic markers (FIG. 15C). Notably, CER T cells recovered from the peritoneal cavity of tumor-bearing mice exhibited upregulation of Xbp1s and the canonical XBP1s-target genes Sec61a1a and ERdj4, with concomitant Tagln2 repression, compared with their counterparts prior to infusion (FIG. 15D). Importantly, CER T cells isolated from the peritoneal cavity and metastatic omental lesions demonstrated a time-dependent reduction in TAGLN2 and FABP5 surface levels after transfer (FIG. 15E, FIG. 15F), which was accompanied by a progressive decline in their expression of IFN-γ and Granzyme B (FIG. 15G, FIG. 15H). We therefore tested whether ER stress-driven repression of TAGLN2 compromised the tumoricidal activity of CER T cells. Tunicamycin-induced ER stress inhibited Tagln2 expression in CER T cells (FIG. 15I, FIG. 15J) and drastically reduced their cytotoxic capacity towards PPNM OvCa cells (FIG. 6A and FIG. 15K, FIG. 15L). Strikingly, the killing capacity of ER-stressed CER T cells was restored upon electroporation with Tagln2 but not control mRNAs (FIG. 6A). Hence, we surmised that preserving TAGLN2 expression in CER T cells could be used to enhance their therapeutic effects in the hostile OvCa microenvironment.

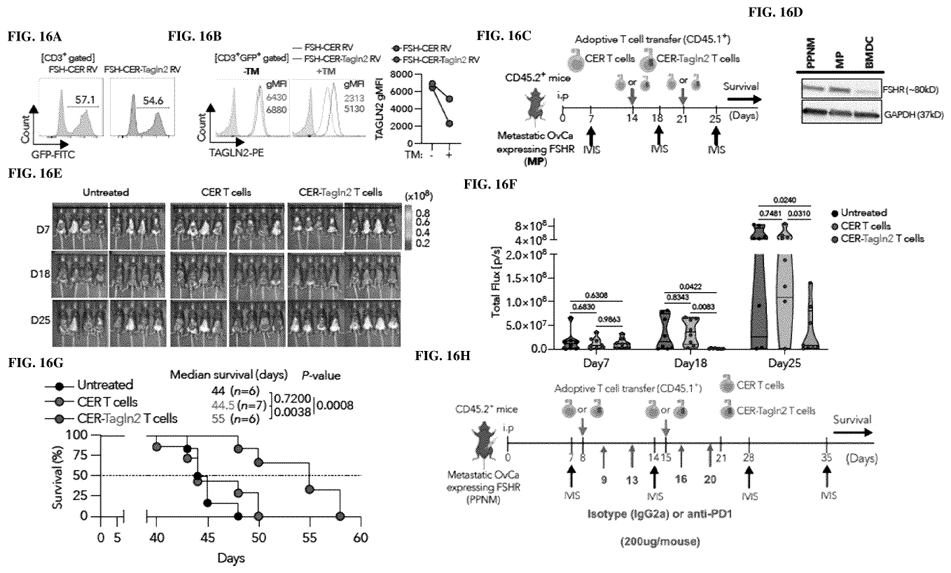

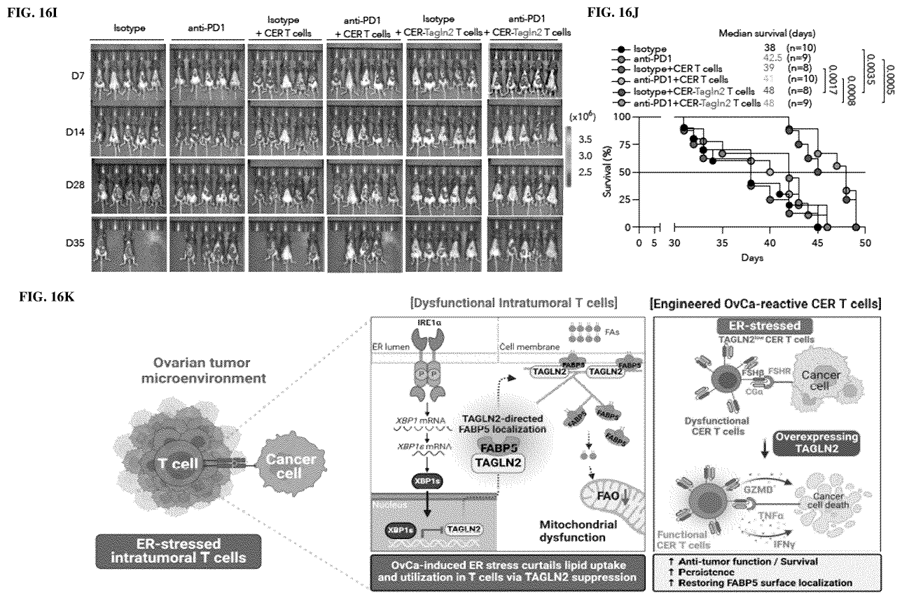

We subcloned the Tagln2 cDNA downstream of the CER construct (CER-Tagln2 RV, FIG. 6B). This enabled simultaneous and constitutive expression of the CER and TAGLN2 upon transduction, and the maintenance of high TAGLN2 levels in T cells facing ER stress (FIG. 16A, FIG. 16B). Thus, we next evaluated the status and therapeutic efficacy of TAGLN2-overexpressing CER T cells in mice bearing PPNM-based HGSC (FIG. 6C). Immunophenotyping analyses conducted 7 days after the second T cell infusion (day 21 of tumor development) demonstrated that CER-Tagln2 T cells infiltrating the omentum and peritoneal cavity maintained high TAGLN2 expression, while unmodified CER T cells present at the same tumor sites exhibited minimal production of this cytoskeletal element (FIG. 6D). Of note, preserving TAGLN2 expression increased the proportion of central memory (CD44⁺CD62L⁺) CD8+CER T cells in the peritoneal cavity and omentum (FIG. 6E) while augmenting their surface levels of FABP5 (FIG. 6F) and capacity to produce IFN-γ, TNF-α, and Granzyme B (FIG. 6G-FIG. 6I). Accordingly, TAGLN2-overexpressing CER T cells demonstrated improved ability to control metastatic HGSC progression, as evidenced by reduced peritoneal carcinomatosis (FIG. 6J, FIG. 6K) and enhanced T cell infiltration into metastatic omental lesions (FIG. 6L, FIG. 6M), compared with their control counterparts devoid of TAGLN2. Adoptive immunotherapy using unmodified CER T cells failed to confer a survival benefit in mice developing these aggressive tumors (FIG. 6N). Strikingly, however, treatment with TAGLN2-overpressing CER T cells significantly prolonged the overall survival of mice with metastatic disease (FIG. 6N). Validating these effects in an independent model of HGSOC, mice bearing peritoneal ovarian carcinomatosis derived from autochthonous MP-based tumors⁵¹ intrinsically expressing the FSHR (FIG. 16D) also demonstrated delayed metastatic progression and increased overall survival upon administration of TAGLN2-overexpressing CER T cells, compared with unmodified CER T cells (FIG. 16E-FIG. 16G). PD-1 blockade did not alter the therapeutic effects of TAGLN2-overexpressing CER T cells, indicating that their enhanced anti-OvCa activity proceeds independently of this immune checkpoint. (FIG. 16H-FIG. 16J). We therefore conclude that advanced ovarian tumors limit the protective capacity of adoptively transferred CER T cells by abrogating TAGLN2, and that preserving the expression of this critical cytoskeletal element enables CER T cells to bypass the detrimental effects of tumor-induced ER stress, conferring improved therapeutic effects against metastatic disease.

Tumors create hostile microenvironments that impede the development and maintenance of effective anti-cancer immunity. Nonetheless, how intratumoral immune cells integrate and interpret persistent stress signals in this harsh milieu remains incompletely understood. Our study presents experimental evidence indicating that ER stress responses cripple anti-tumor T cell function by disabling TAGLN2-coordinated immunometabolic programs. We propose that TAGLN2 operates as a central gatekeeper of CD8⁺ T cell lipid metabolic programming by enabling FABP5-driven import of fatty acids that fuel T cell mitochondrial respiration and effector function (FIG. 16K—proposed model). Preserving the TAGLN2-FABP5 metabolic axis therefore represents a major opportunity to improve the effects of T cell-based immunotherapies in aggressive solid tumors such as metastatic OvCa.

### REFERENCES

1 Lim, S. A., Su, W., Chapman, N. M. & Chi, H. Lipid metabolism in T cell signaling and function. Nature Chemical Biology 18, 470-481 (2022).
2 Zhang, Y. et al. Enhancing CD8(+) T Cell Fatty Acid Catabolism within a Metabolically Challenging Tumor Microenvironment Increases the Efficacy of Melanoma Immunotherapy. Cancer Cell 32, 377-391 e379 (2017).
3 Nava Lauson, C. B. et al. Linoleic acid potentiates CD8(+) T cell metabolic fitness and antitumor immunity. Cell Metab 35, 633-650.e639 (2023).
4 Pan, Y. et al. Survival of tissue-resident memory T cells requires exogenous lipid uptake and metabolism. Nature 543, 252-256 (2017).
5 Lin, R. et al. Fatty Acid Oxidation Controls CD8(+) Tissue-Resident Memory T-cell Survival in Gastric Adenocarcinoma. Cancer Immunol Res 8, 479-492 (2020).
6 Reinfeld, B. I. et al. Cell-programmed nutrient partitioning in the tumour microenvironment. Nature 593, 282-288 (2021).
7 Long, L. et al. CRISPR screens unveil signal hubs for nutrient licensing of T cell immunity. Nature 600, 308-313 (2021).
8 Fox, C. J., Hammerman, P. S. & Thompson, C. B. Fuel feeds function: energy metabolism and the T-cell response. Nat Rev Immunol 5, 844-852 (2005).
9 Byersdorfer, C. A. et al. Effector T cells require fatty acid metabolism during murine graft-versus-host disease. Blood 122, 3230-3237 (2013).
10 Pearce, E. L. et al. Enhancing CD8 T-cell memory by modulating fatty acid metabolism. Nature 460, 103-107 (2009).
11 Rolph, M. S. et al. Regulation of dendritic cell function and T cell priming by the fatty acid-binding protein AP2. J Immunol 177, 7794-7801 (2006).
12 Kim, N. et al. Single-cell RNA sequencing demonstrates the molecular and cellular reprogramming of metastatic lung adenocarcinoma. Nat Commun 11, 2285 (2020).
13 Liu, F. et al. Identification of FABP5 as an immunometabolic marker in human hepatocellular carcinoma. J Immunother Cancer 8 (2020).
14 Matulonis, U. A. et al. Ovarian cancer. Nat Rev Dis Primers 2, 16061 (2016).
15 Matulonis, U. A. et al. Antitumor activity and safety of pembrolizumab in patients with advanced recurrent ovarian cancer: results from the phase II KEYNOTE-100 study. Ann Oncol 30, 1080-1087 (2019).
16 Kandalaft, L. E., Dangaj Laniti, D. & Coukos, G. Immunobiology of high-grade serous ovarian cancer: lessons for clinical translation. Nat Rev Cancer 22, 640-656 (2022).
17 Song, M. et al. IRE1alpha-XBP1 controls T cell function in ovarian cancer by regulating mitochondrial activity. Nature 562, 423-428 (2018).
18 Cao, Y. et al. ER stress-induced mediator C/EBP homologous protein thwarts effector T cell activity in tumors through T-bet repression. Nat Commun 10, 1280 (2019).
19 Anadon, C. M. et al. Ovarian cancer immunogenicity is governed by a narrow subset of progenitor tissue-resident memory T cells. Cancer Cell 40, 545-557 e513 (2022).
20 Bowtell, D. D. The genesis and evolution of high-grade serous ovarian cancer. Nat Rev Cancer 10, 803-808 (2010).
21 Digre, A. & Lindskog, C. The Human Protein Atlas-Spatial localization of the human proteome in health and disease. Protein Sci 30, 218-233 (2021).
22 Conejo-Garcia, J. R. et al. Tumor-infiltrating dendritic cell precursors recruited by a beta-defensin contribute to vasculogenesis under the influence of Vegf-A. Nat Med 10, 950-958 (2004).
23 Chae, C. S. et al. Tumor-derived Lysophosphatidic Acid Blunts Protective Type-I Interferon Responses in Ovarian Cancer. Cancer Discov (2022).
24 Cubillos-Ruiz, J. R. et al. Reprogramming tumor-associated dendritic cells in vivo using miRNA mimetics triggers protective immunity against ovarian cancer. Cancer Res 72, 1683-1693 (2012).
25 Cubillos-Ruiz, J. R. et al. Polyethylenimine-based siRNA nanocomplexes reprogram tumor-associated dendritic cells via TLR5 to elicit therapeutic antitumor immunity. J Clin Invest 119, 2231-2244 (2009).
26 Cubillos-Ruiz, J. R. et al. E R Stress Sensor XBP1 Controls Anti-tumor Immunity by Disrupting Dendritic Cell Homeostasis. Cell 161, 1527-1538 (2015).
27 Scarlett, U. K. et al. Ovarian cancer progression is controlled by phenotypic changes in dendritic cells. J Exp Med 209, 495-506 (2012).
28 Szklarczyk, D. et al. The STRING database in 2021: customizable protein-protein networks, and functional characterization of user-uploaded gene/measurement sets. Nucleic Acids Res 49, D605-D612 (2021).
29 Wan, C. H. et al. Panorama of ancient metazoan macromolecular complexes. Nature 525, 339-+(2015).
30 Yin, L. M., Ulloa, L. & Yang, Y. Q. Transgelin-2: Biochemical and Clinical Implications in Cancer and Asthma. Trends Biochem Sci 44, 885-896 (2019).
31 Jo, S., Kim, H. R., Mun, Y. & Jun, C. D. Transgelin-2 in immunity: Its implication in cell therapy. J Leukoc Biol 104, 903-910 (2018).
32 Ortega, F. J. et al. Cytoskeletal transgelin 2 contributes to gender-dependent adipose tissue expandability and immune function. FASEB J 33, 9656-9671 (2019).
33 Na, B. R. et al. TAGLN2 regulates T cell activation by stabilizing the actin cytoskeleton at the immunological synapse. J Cell Biol 209, 143-162 (2015).
34 Armstrong, E. H., Goswami, D., Griffin, P. R., Noy, N. & Ortlund, E. A. Structural basis for ligand regulation of the fatty acid-binding protein 5, peroxisome proliferator-activated receptor beta/delta (FABP5-PPARbeta/delta) signaling pathway. J Biol Chem 289, 14941-14954 (2014).
35 Fung, H. Y., Teryek, M., Lemenze, A. D. & Bergsbaken, T. CD103 fate mapping reveals that intestinal CD103(−) tissue-resident memory T cells are the primary responders to secondary infection. Sci Immunol 7, eab19925 (2022).
36 Giles, J. R. et al. Shared and distinct biological circuits in effector, memory and exhausted CD8(+) T cells revealed by temporal single-cell transcriptomics and epigenetics. Nat Immunol 23, 1600-1613 (2022).
37 Tirosh, I. et al. Dissecting the multicellular ecosystem of metastatic melanoma by single-cell RNA-seq. Science 352, 189-196 (2016).
38 Scott, A. C. et al. TOX is a critical regulator of tumour-specific T cell differentiation. Nature 571, 270-274 (2019).
39 Hetz, C., Zhang, K. & Kaufman, R. J. Mechanisms, regulation and functions of the unfolded protein response. Nat Rev Mol Cell Biol 21, 421-438 (2020).
40 Chen, X. & Cubillos-Ruiz, J. R. Endoplasmic reticulum stress signals in the tumour and its microenvironment. Nat Rev Cancer 21, 71-88 (2021).
41 Yoshida, H., Matsui, T., Yamamoto, A., Okada, T. & Mori, K. XBP1 mRNA is induced by ATF6 and spliced by IRE1 in response to ER stress to produce a highly active transcription factor. Cell 107, 881-891 (2001).
42 Logue, S. E. et al. Inhibition of IRE1 RNase activity modulates the tumor cell secretome and enhances response to chemotherapy. Nat Commun 9, 3267 (2018).
43 Morita, S. et al. Targeting ABL-IRE1alpha Signaling Spares ER-Stressed Pancreatic beta Cells to Reverse Autoimmune Diabetes. Cell Metab 25, 1207 (2017).
44 Di Conza, G., Ho, P. C., Cubillos-Ruiz, J. R. & Huang, S. C. Control of immune cell function by the unfolded protein response. Nat Rev Immunol 23, 546-562 (2023).
45 Kim, H. R. et al. An Essential Role for TAGLN2 in Phagocytosis of Lipopolysaccharide-activated Macrophages. Sci Rep 7, 8731 (2017).
46 Iyer, S. et al. Genetically Defined Syngeneic Mouse Models of Ovarian Cancer as Tools for the Discovery of Combination Immunotherapy. Cancer Discov 11, 384-407 (2021).
47 Zhang, K. Y. et al. Longitudinal single-cell RNA-seq analysis reveals stress-promoted chemoresistance in metastatic ovarian cancer. Science Advances 8 (2022).
48 Snaebjornsson, M. T., Janaki-Raman, S. & Schulze, A. Greasing the Wheels of the Cancer Machine: The Role of Lipid Metabolism in Cancer. Cell Metab 31, 62-76 (2020).
49 Shender, V. O. et al. Proteome-metabolome profiling of ovarian cancer ascites reveals novel components involved in intercellular communication. Mol Cell Proteomics 13, 3558-3571 (2014).
50 Perales-Puchalt, A. et al. Follicle-Stimulating Hormone Receptor Is Expressed by Most Ovarian Cancer Subtypes and Is a Safe and Effective Immunotherapeutic Target. Clin Cancer Res 23, 441-453 (2017).
51 Paffenholz, S. V. et al. Senescence induction dictates response to chemo- and immunotherapy in preclinical models of ovarian cancer. Proc Natl Acad Sci USA 119 (2022).
52 Bogdan, D. M. et al. FABP5 deletion in nociceptors augments endocannabinoid signaling and suppresses TRPV1 sensitization and inflammatory pain. Sci Rep 12, 9241 (2022).
53 Lee, A. H., Scapa, E. F., Cohen, D. E. & Glimcher, L. H. Regulation of hepatic lipogenesis by the transcription factor XBP1. Science 320, 1492-1496 (2008).
54 Iwawaki, T., Akai, R., Yamanaka, S. & Kohno, K. Function of IRE1 alpha in the placenta is essential for placental development and embryonic viability. Proc Natl Acad Sci USA 106, 16657-16662 (2009).
55 Chopra, S. et al. IRE1alpha-XBP1 signaling in leukocytes controls prostaglandin biosynthesis and pain. Science 365 (2019).
56 de Boer, J. et al. Transgenic mice with hematopoietic and lymphoid specific expression of Cre. Eur J Immunol 33, 314-325 (2003).
57 Roby, K. F. et al. Development of a syngeneic mouse model for events related to ovarian cancer. Carcinogenesis 21, 585-591 (2000).
58 Bolte, S. & Cordelieres, F. P. A guided tour into subcellular colocalization analysis in light microscopy. J Microsc 224, 213-232 (2006).
59 Hao, Y. et al. Integrated analysis of multimodal single-cell data. Cell 184, 3573-3587 e3529 (2021).
60 Andreatta, M. et al. Interpretation of T cell states from single-cell transcriptomics data using reference atlases. Nat Commun 12, 2965 (2021).
61 Wu, T. et al. clusterProfiler 4.0: A universal enrichment tool for interpreting omics data. Innovation (Camb) 2, 100141 (2021).

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

Also incorporated by reference in their entirety are any polynucleotide and polypeptide sequences which reference an accession number correlating to an entry in a public database, such as those maintained by The Institute for Genomic Research (TIGR) on the world wide web at tigr.org and/or the National Center for Biotechnology Information (NCBI) on the World Wide Web at ncbi.nlm.nih.gov.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method of treating a subject afflicted with a cancer, comprising administering to the subject an agent that increases expression, level, and/or activity of Transgelin-2 (TAGLN2) in T cells.

2. The method of claim 1, wherein the T cells are CD8⁺ T cells, intratumoral T cells, and/or ER-stressed T cells.

3.-6. (canceled)

7. The method of claim 1, wherein the cancer is metastatic ovarian cancer (OvCa).

8. (canceled)

9. The method of claim 1, wherein prior to the administration of the agent, the T cells have one or more properties selected from: (1) a reduced level of TAGLN2 compared to T cells from a healthy subject or a subject without the cancer: (2) a reduced surface level of FABP5 and/or reduced lipid uptake compared to T cells from a healthy subject or a subject without the cancer; and (3) an increased expression of Xbp1s, Sec61a1a, and ERdj4 compared to T cells from a healthy subject or a subject without the cancer.

10.-11. (canceled)

12. A method of increasing cytotoxicity of a T cell comprising contacting the T cell with an agent that increases expression, level, and/or activity of Transgelin-2 (TAGLN2) in the T cell.

13. The method of claim 12, wherein the T cell expresses a chimeric antigen receptor (CAR).

14. The method of claim 13, wherein the CAR is a chimeric endocrine receptor (CER).

15. The method of claim 14, wherein the chimeric endocrine receptor (CER) comprises: a) the β subunit of the follicle-stimulating hormone (FSHβ), b) a spacer, c) a transmembrane domain, and d) an intracellular domain.

16.-17. (canceled)

18. The method of claim 15, wherein the transmembrane domain is a CD8α transmembrane domain and/or the intracellular domain is a 4-1BB-CD3ζ (BBZ) intracellular domain (ICD).

19. (canceled)

20. The method of claim 12, wherein the T cell is a cytotoxic T cell.

21.-24. (canceled)

25. The method of claim 12, wherein the cancer is metastatic ovarian cancer (OvCa).

26.-32. (canceled)

33. The method of claim 1, wherein the agent is a Tagln2 mRNA or a TAGLN2 protein.

34.-55. (canceled)

56. A T cell expressing a chimeric antigen receptor (CAR) and comprising a transgene encoding TAGLN2.

57. The T cell of claim 56, wherein the CAR is a chimeric endocrine receptor (CER).

58. The T cell of claim 57, wherein the chimeric endocrine receptor (CER) comprises: a) the β subunit of the follicle-stimulating hormone (FSHβ), b) a spacer, c) a transmembrane domain, and d) an intracellular domain.

59.-60. (canceled)

61. The T cell of claim 58, wherein the transmembrane domain is a CD8α transmembrane domain and/or the intracellular domain is a 4-1BB-CD3ζ (BBZ) intracellular domain (ICD).

62. (canceled)

63. The T cell of claim 56, wherein the T cell expresses TAGLN2.

64. (canceled)

65. The T cell of claim 56, wherein the T cell is a cytotoxic T lymphocyte (CTL).

66. (canceled)

67. A composition comprising T cells of claim 56.

68. (canceled)

69. A method of treating a cancer in a subject, the method comprising administering a composition comprising T cells of claim 56.

70.-100. (canceled)


SEQUENCE LISTING (47 sequences)

SEQ ID NO: 1 - DNA, 1616 bp, Homo sapiens
  source 1..1616: mol_type = genomic DNA
cttctgctcctgactctcggtggtgaggggctgccctacggtggttcccttaattgtgggccgccccccatccttgctgctggctgtggcacctcgctctgtgggctggtgccctcactgtgctgctctttgagaacactccctgtcccaaatctgagttgggaagttctcctgtgttccatctgcacggtggatggagagtccctttgtcttcccaattaaacaccctagccaagaaccctgaagtcctccctgcccccacccatgtccgccttttctttggctttggctttggtgagctccccgcagccaccgccacccattggaatggccaacaggggacctgcatatggcctgagccgggaggtgcagcagaagattgagaaacaatatgatgcagatctggagcagatcctgatccagtggatcaccacccagtgccgaaaggatgtgggccggccccagcctggacgcgagaacttccagaactggctcaaggatggcacggtgctatgtgagctcattaatgcactgtaccccgaggggcaggccccagtaaagaagatccaggcctccaccatggccttcaagcagatggagcagatctctcagttcctgcaagcagctgagcgctatggcattaacaccactgacatcttccaaactgtggacctctgggaaggaaagaacatggcctgtgtgcagcggacgctgatgaatctgggtgggctggcagtagcccgagatgatgggctcttctctggggatcccaactggttccctaagaaatccaaggagaatcctcggaacttctcggataaccagctgcaagagggcaagaacgtgatcgggttacagatgggcaccaaccgcggggcgtctcaggcaggcatgactggctacgggatgccacgccagatcctctgatcccaccccaggccttgcccctgccctcccacgaatggttaatatatatgtagatatatattttagcagtgacattcccagagagccccagagctctcaagctcctttctgtcagggtggggggttcagcctgtcctgtcacctctgaggtgcctgctggcatcctctcccccatgcttactaatacattcccttccccatagccatcaaaactggaccaactggcctcttcctttcccctgggaccaaaatttaggggcctcagtccctcaccgccatgccctggcctattctgtctctccttcttccccctggcctgttctgtctctgagctctgtgtcctccgttcattccatggctgggagtcactgatgctgcctctgccttctgatgctggactggccttgcttctacaagtatgcttctcccacagctgtggctgcaggaacttaatttatagggaggagcctgtggcagctgctgccccagccacagctgcactgactgtgctcaccacacatctggggcagccttccctggcaggggccctcgtggcttctcattttccattcccttcactgtggctaaggggtggggtgaggggatggagagggagggctgcctaccatggtctggggcttgaggaagatgagtttgttgatttaaataaagaatttgtcatttttgaa

SEQ ID NO: 2 - DNA, 1375 bp, Homo sapiens
  source 1..1375: mol_type = genomic DNA
acttgcagctgcagcccttgccttgagtcagtgcgccgctctccagcccgcttgaacgctccccgcagccaccgccacccattggaatggccaacaggggacctgcatatggcctgagccgggaggtgcagcagaagattgagaaacaatatgatgcagatctggagcagatcctgatccagtggatcaccacccagtgccgaaaggatgtgggccggccccagcctggacgcgagaacttccagaactggctcaaggatggcacggtgctatgtgagctcattaatgcactgtaccccgaggggcaggccccagtaaagaagatccaggcctccaccatggccttcaagcagatggagcagatctctcagttcctgcaagcagctgagcgctatggcattaacaccactgacatcttccaaactgtggacctctgggaaggaaagaacatggcctgtgtgcagcggacgctgatgaatctgggtgggctggcagtagcccgagatgatgggctcttctctggggatcccaactggttccctaagaaatccaaggagaatcctcggaacttctcggataaccagctgcaagagggcaagaacgtgatcgggttacagatgggcaccaaccgcggggcgtctcaggcaggcatgactggctacgggatgccacgccagatcctctgatcccaccccaggccttgcccctgccctcccacgaatggttaatatatatgtagatatatattttagcagtgacattcccagagagccccagagctctcaagctcctttctgtcagggtggggggttcagcctgtcctgtcacctctgaggtgcctgctggcatcctctcccccatgcttactaatacattcccttccccatagccatcaaaactggaccaactggcctcttcctttcccctgggaccaaaatttaggggcctcagtccctcaccgccatgccctggcctattctgtctctccttcttccccctggcctgttctgtctctgagctctgtgtcctccgttcattccatggctgggagtcactgatgctgcctctgccttctgatgctggactggccttgcttctacaagtatgcttctcccacagctgtggctgcaggaacttaatttatagggaggagcctgtggcagctgctgccccagccacagctgcactgactgtgctcaccacacatctggggcagccttccctggcaggggccctcgtggcttctcattttccattcccttcactgtggctaaggggtggggtgaggggatggagagggagggctgcctaccatggtctggggcttgaggaagatgagtttgttgatttaaataaagaatttgtcatttttgaa

SEQ ID NO: 3 - DNA, 1419 bp, Homo sapiens
  source 1..1419: mol_type = genomic DNA
ggcggctgggaggccggacctgcaggaccggaggtggactgccgcctccccccgcgccctgtgcctcctggggctacctgaagccggtgtcctgggagatctctccccgcagccaccgccacccattggaatggccaacaggggacctgcatatggcctgagccgggaggtgcagcagaagattgagaaacaatatgatgcagatctggagcagatcctgatccagtggatcaccacccagtgccgaaaggatgtgggccggccccagcctggacgcgagaacttccagaactggctcaaggatggcacggtgctatgtgagctcattaatgcactgtaccccgaggggcaggccccagtaaagaagatccaggcctccaccatggccttcaagcagatggagcagatctctcagttcctgcaagcagctgagcgctatggcattaacaccactgacatcttccaaactgtggacctctgggaaggaaagaacatggcctgtgtgcagcggacgctgatgaatctgggtgggctggcagtagcccgagatgatgggctcttctctggggatcccaactggttccctaagaaatccaaggagaatcctcggaacttctcggataaccagctgcaagagggcaagaacgtgatcgggttacagatgggcaccaaccgcggggcgtctcaggcaggcatgactggctacgggatgccacgccagatcctctgatcccaccccaggccttgcccctgccctcccacgaatggttaatatatatgtagatatatattttagcagtgacattcccagagagccccagagctctcaagctcctttctgtcagggtggggggttcagcctgtcctgtcacctctgaggtgcctgctggcatcctctcccccatgcttactaatacattcccttccccatagccatcaaaactggaccaactggcctcttcctttcccctgggaccaaaatttaggggcctcagtccctcaccgccatgccctggcctattctgtctctccttcttccccctggcctgttctgtctctgagctctgtgtcctccgttcattccatggctgggagtcactgatgctgcctctgccttctgatgctggactggccttgcttctacaagtatgcttctcccacagctgtggctgcaggaacttaatttatagggaggagcctgtggcagctgctgccccagccacagctgcactgactgtgctcaccacacatctggggcagccttccctggcaggggccctcgtggcttctcattttccattcccttcactgtggctaaggggtggggtgaggggatggagagggagggctgcctaccatggtctggggcttgaggaagatgagtttgttgatttaaataaagaatttgtcatttttgaa

SEQ ID NO: 4 - protein, 220 aa, Homo sapiens
MSAFSLALALVSSPQPPPPIGMANRGPAYGLSREVQQKIEKQYDADLEQILIQWITTQCRKDVGRPQPGRENFQNWLKDGTVLCELINALYPEGQAPVKKIQASTMAFKQMEQISQFLQAAERYGINTTDIFQTVDLWEGKNMACVQRTLMNLGGLAVARDDGLFSGDPNWFPKKSKENPRNFSDNQLQEGKNVIGLQMGTNRGASQAGMTGYGMPRQIL

SEQ ID NO: 5 - protein, 199 aa, Homo sapiens
MANRGPAYGLSREVQQKIEKQYDADLEQILIQWITTQCRKDVGRPQPGRENFQNWLKDGTVLCELINALYPEGQAPVKKIQASTMAFKQMEQISQFLQAAERYGINTTDIFQTVDLWEGKNMACVQRTLMNLGGLAVARDDGLFSGDPNWFPKKSKENPRNFSDNQLQEGKNVIGLQMGTNRGASQAGMTGYGMPRQIL

SEQ ID NO: 6 - DNA, 1382 bp, Mus musculus
  source 1..1382: mol_type = genomic DNA
gaaaactccaagcccggccgggtcttgagctccactcgccgctgcagcccctgtcgtgcgtgcgctctcatccagccctcttggacgctctttgccatcaccacagctgctcagaatggccaacaggggaccttcctacggcctgagccgagaggtgcagcagaagattgagaagcagtacgacgcggatctggagcagatcctcatccagtggatcaccactcagtgccgtgaggacgtgggccagccccagcctggccgtgagaacttccagaagtggctcaaggacggcacggttctgtgcaagcttattaattcactgtatcctgaggggcaggccccagtaaagaagatccaggcctcttcgatggccttcaagcagatggagcagatctcccagttcctgcaggcagccgagcgctatggcattaacaccacggacatcttccagactgtggatctctgggaaggaaagaacatggcttgtgtgcagcggacactaatgaacctgggtgggctggcagtagccagggacgatgggctcttctctggggatcccaactggtttcctaagaaatccaaggagaaccctcggaacttctcggacaaccagttgcaagagggcaagaacgtgattgggttgcagatgggcaccaaccgtggagcatctcaggccggcatgaccggctatgggatgccacggcagatcctctgatcatactctctctccttcccctgccctccatgaatggttaatatatatgtatatatatgttttagcagacattccctgagagcccctggattgctgaactcccctctgccagggtccaggccagcctatcttgtcaccactggcagggcctgataattgcctctctctctctctctctttctctctctctctctctctctctctctctctctctctctctgggcttactaatgcattccttcccccacaaccatcaaaactggaccaacaaaaaccctgggaccaaagttgcctccccacagcatctttcctgctttcctgatctcttcttttagtccatcccttggctaggagtcagagattctgccccatggcctgatgctgcaccgacccttccttctacaaggaggcctctcctacagctgtggctgcagggacttaatttatagggaggggcctgtggctgtcactccagccacagctgggctgtacttaccacacgtctgggcagctttccctagcagaggctctttggcttctttctttccattcctctctcactgtggctaaggggtggagcagaggtaggacggctgcccaccatgctctggggcttgacgaacacgagtttgctgattttaaataaaaagatctcattttgttttgc

SEQ ID NO: 7 - protein, 199 aa, Mus musculus
MANRGPSYGLSREVQQKIEKQYDADLEQILIQWITTQCREDVGQPQPGRENFQKWLKDGTVLCKLINSLYPEGQAPVKKIQASSMAFKQMEQISQFLQAAERYGINTTDIFQTVDLWEGKNMACVQRTLMNLGGLAVARDDGLFSGDPNWFPKKSKENPRNFSDNQLQEGKNVIGLQMGTNRGASQAGMTGYGMPRQIL

SEQ ID NO: 8 - DNA, 739 bp, Mus musculus
  source 1..739: mol_type = genomic DNA
cacccctcaacttattgctcttcactcttaaggggtagaaaagtgcctgccaacttcctccactaagcccccgcctccaaccctccaagatctaagaagaaatgtcaatttattaaatgggagatggaagggagagagagaactgggtctgaatcccaggtcccaagccctaccacaccctcaaggccccgggcaaggagtgacctcactgttctttttttttttttgcccgggcttgaagttaaatggcaagcagaaccacgtgggggtgctgtcccagcctgtaagcaccagacctattactccttactctatcctgtaggaaggcgcttcctggacctgaatttacaaaaggccggcctttttgtaatagtaggcttctcatagaaacaggcctgcgatcctgagctagctagtctaggcagaaagtttcctattaggatacaagtctatggctggtgaatgcaggttgaggatcccctgggcggagaccttttgcagcggcgccaaggactccgcaggtcctttacggctcattcctgcctactgaccacctatcttaagcgctccagcattccacgcggcggcgcctttaaaccctcttggcccttgccccgcccctggtccgccccgtctgaggcgggaccccccagaaaactccaagcccggccgggtcttgagctccactcgccgctgcagcccctgtcgtgcgtgcgctctcatccagccctcttggacg

SEQ ID NO: 9 - protein, 9 aa, synthetic construct
GGGGAGGGG

SEQ ID NO: 10 - DNA, 20 bp, synthetic construct
  source 1..20: mol_type = other DNA
gcgagaagatgacccagatc

SEQ ID NO: 11 - DNA, 20 bp, synthetic construct
  source 1..20: mol_type = other DNA
atggcacggtgctatgtgag

SEQ ID NO: 12 - DNA, 18 bp, synthetic construct
  source 1..18: mol_type = other DNA
ctgagtccgcagcaggtg

SEQ ID NO: 13 - DNA, 21 bp, synthetic construct
  source 1..21: mol_type = other DNA
tgaaggagctaggagtgggaa

SEQ ID NO: 14 - DNA, 22 bp, synthetic construct
  source 1..22: mol_type = other DNA
aagccaggtattgcagttcttt

SEQ ID NO: 15 - DNA, 20 bp, synthetic construct
  source 1..20: mol_type = other DNA
actgggccaggaatttgacg

SEQ ID NO: 16 - DNA, 23 bp, synthetic construct
  source 1..23: mol_type = other DNA
tcggtaactgacttgaatgtcca

SEQ ID NO: 17 - DNA, 20 bp, synthetic construct
  source 1..20: mol_type = other DNA
ggagaagggtgaccgactca

SEQ ID NO: 18 - DNA, 21 bp, synthetic construct
  source 1..21: mol_type = other DNA
ccctgggaaaacactcacaca

SEQ ID NO: 19 - DNA, 24 bp, synthetic construct
  source 1..24: mol_type = other DNA
ctcaggaggagcaatgatcttgat

SEQ ID NO: 20 - DNA, 20 bp, synthetic construct
  source 1..20: mol_type = other DNA
cttgaggctcaccacaggaa

SEQ ID NO: 21 - DNA, 30 bp, synthetic construct
  source 1..30: mol_type = other DNA
tctttgccatcaccacagctgctcagaatg

SEQ ID NO: 22 - DNA, 20 bp, synthetic construct
  source 1..20: mol_type = other DNA
aagaacacgcttgggaatgg

SEQ ID NO: 23 - DNA, 21 bp, synthetic construct
  source 1..21: mol_type = other DNA
ctatttccagggcttccgagt

SEQ ID NO: 24 - DNA, 21 bp, synthetic construct
  source 1..21: mol_type = other DNA
taaaagccctgatgctgaagc

SEQ ID NO: 25 - DNA, 28 bp, synthetic construct
  source 1..28: mol_type = other DNA
ggggtacccccacccctcaactattgct

SEQ ID NO: 26 - DNA, 20 bp, synthetic construct
  source 1..20: mol_type = other DNA
aggggtagaaaagtgcctgc

SEQ ID NO: 27 - DNA, 23 bp, synthetic construct
  source 1..23: mol_type = other DNA
agttaaatggcaagcagaaccac

SEQ ID NO: 28 - DNA, 20 bp, synthetic construct
  source 1..20: mol_type = other DNA
ttcctgcctactgaccacct

SEQ ID NO: 29 - DNA, 19 bp, synthetic construct
  source 1..19: mol_type = other DNA
ccagtggtacggccagagg

SEQ ID NO: 30 - DNA, 20 bp, synthetic construct
  source 1..20: mol_type = other DNA
cccacccagattcatcagcg

SEQ ID NO: 31 - DNA, 20 bp, synthetic construct
  source 1..20: mol_type = other DNA
tccaagttgtccagaatgcc

SEQ ID NO: 32 - DNA, 22 bp, synthetic construct
  source 1..22: mol_type = other DNA
tgcaccatctgtaaagttgcag

SEQ ID NO: 33 - DNA, 22 bp, synthetic construct
  source 1..22: mol_type = other DNA
gcatttgctgatgtctagcaca

SEQ ID NO: 34 - DNA, 19 bp, synthetic construct
  source 1..19: mol_type = other DNA
ctcgtggaagtgacgcctt

SEQ ID NO: 35 - DNA, 21 bp, synthetic construct
  source 1..21: mol_type = other DNA
tcgcttccctgttttagctgc

SEQ ID NO: 36 - DNA, 17 bp, synthetic construct
  source 1..17: mol_type = other DNA
ctgcccagactcggcaa

SEQ ID NO: 37 - DNA, 22 bp, synthetic construct
  source 1..22: mol_type = other DNA
gcacaactcaatggtactgtcg

SEQ ID NO: 38 - DNA, 20 bp, synthetic construct
  source 1..20: mol_type = other DNA
taccaccatgtacccaggca

SEQ ID NO: 39 - DNA, 20 bp, synthetic construct
  source 1..20: mol_type = other DNA
ttgaaggccatcgaagaggc

SEQ ID NO: 40 - DNA, 30 bp, synthetic construct
  source 1..30: mol_type = other DNA
cgtgccgtccttgagccacttctggaagtt

SEQ ID NO: 41 - DNA, 17 bp, synthetic construct
  source 1..17: mol_type = other DNA
ctgcacctgctgcggac

SEQ ID NO: 42 - DNA, 21 bp, synthetic construct
  source 1..21: mol_type = other DNA
aggtgttgtactggcctcggt

SEQ ID NO: 43 - DNA, 19 bp, synthetic construct
  source 1..19: mol_type = other DNA
tccgactattggcatccga

SEQ ID NO: 44 - DNA, 28 bp, synthetic construct
  source 1..28: mol_type = other DNA
ccgctcgagcggcgtccaagagggctgg

SEQ ID NO: 45 - DNA, 20 bp, synthetic construct
  source 1..20: mol_type = other DNA
cagtgaggtcactccttgcc

SEQ ID NO: 46 - DNA, 23 bp, synthetic construct
  source 1..23: mol_type = other DNA
gcgccttcctacaggatagagta

SEQ ID NO: 47 - DNA, 20 bp, synthetic construct
  source 1..20: mol_type = other DNA
gcaagggccaagagggttta